(19) United States
(12) Patent Application Publication
(10) Pub. No.: US 2024/0084359 A1
(43) Pub. Date: Mar. 14, 2024

(54) METHODS AND COMPOSITIONS FOR PATTERNED MOLECULAR ARRAY GENERATION BY DIRECTED BEAD DELIVERY

(71) Applicant: 10x Genomics, Inc., Pleasanton, CA (US)

(72) Inventors: Michael SCHNALL-LEVIN, San Francisco, CA (US); Preyas SHAH, Pleasanton, CA (US)

(21) Appl. No.: 18/342,891

(22) Filed: Jun. 28, 2023

Related U.S. Application Data
(60) Provisional application No. 63/356,917, filed on Jun. 29, 2022.

(51) Int. Cl. C12Q 1/6806; B01L 3/00; C12Q 1/6874; C12Q 1/6888

(52) U.S. Cl. CPC C12Q 1/6806; B01L 3/50857; C12Q 1/6874; C12Q 1/6888; B01L 2300/0829

(57) ABSTRACT

Provided in some aspects are methods of patterning a surface in situ for producing an array on the surface, for example, by partitioning of beads comprising oligonucleotides into spatially predefined regions, to generate unique DNA sequences in spatial positions in the array. Compositions such as nucleic acid arrays produced by the methods are also disclosed.

### CROSS-REFERENCE TO RELATED APPLICATIONS

This application claims priority to U.S. Provisional Patent Application No. 63/356,917, filed Jun. 29, 2022, entitled “METHODS AND COMPOSITIONS FOR PATTERNED MOLECULAR ARRAY GENERATION BY DIRECTED BEAD DELIVERY,” which is herein incorporated by reference in its entirety for all purposes.

### FIELD

The present disclosure relates in some aspects to molecular arrays and methods for providing molecular arrays in situ on a substrate by directed bead delivery.

### BACKGROUND

Arrays of nucleic acids are an important tool in the biotechnology industry and related fields. These nucleic acid arrays, in which a plurality of distinct or different nucleic acids are positioned on a solid support surface in the form of an array or pattern, find use in a variety of applications, including gene expression analysis, drug screening, nucleic acid sequencing, mutation analysis, and the like.

While nucleic acid arrays have been manufactured using in situ synthesis techniques, those techniques typically utilize nucleotide by nucleotide extension, applications in the field of genomics and high throughput screening have fueled the demand for precise chemistry and high fidelity of the synthesized oligonucleotides. Accordingly, there is continued interest in the development of new methods for producing nucleic acid arrays in situ. Provided herein are methods, uses and articles of manufacture that meet these needs.

### SUMMARY

Nucleic acid arrays in which a plurality of distinct or different nucleic acids are patterned on a solid support surface find use in a variety of applications. A feature of many arrays that have been developed is that each of the distinct nucleic acids of the array is stably attached to a discrete location on the array surface, such that its position remains constant and known throughout the use of the array. Stable attachment is achieved in a number of different ways, including covalent bonding of a nucleic acid polymer to the support surface and non-covalent interaction of the nucleic acid polymer with the surface.

There are two main ways of producing nucleic acid arrays in which the immobilized nucleic acids are covalently attached to the substrate surface, e.g., via in situ synthesis in which the nucleic acid polymer is grown on the surface of the substrate in a step-wise, nucleotide-by-nucleotide fashion, or via deposition of a full, pre-synthesized nucleic acid/polypeptide, cDNA fragment, etc., onto the surface of the array.

Provided are improved methods, uses and articles of manufacture by use of directed bead delivery. In some aspects, provided are methods of patterning spatially defined regions on a surface, forming wells in the corresponding regions, partitioning and disruption of beads comprising oligonucleotides in the wells, to generate extended nucleic acid polymers. Also disclosed are further applications and uses of such in situ generated nucleic acid arrays, e.g., for interrogating analytes in a three-dimensional sample.

In some aspects, disclosed herein are methods for providing an array, which can include (a) partitioning a plurality of Round 1 beads into wells on a substrate, wherein the Round 1 bead in a first well and the Round 1 bead in a second well can each include a different Round 1 oligonucleotide; and (b) disrupting the Round 1 beads to release the Round 1 oligonucleotides, wherein the released Round 1 oligonucleotides can be attached to nucleic acid molecules in the corresponding well via hybridization and/or ligation to generate extended nucleic acid molecules, thereby providing on the substrate an array comprising the extended nucleic acid molecules.

In any of the preceding embodiments, the first and/or second wells can be formed by a material enclosing a first region and a second region, respectively, on the substrate. In any of the preceding embodiments, the first and/or second wells can be formed by etching a layer of the material on the substrate. In any of the preceding embodiments, the nucleic acid molecules can be immobilized on the substrate prior to the etching step, optionally wherein the nucleic acid molecules are immobilized in the first and second regions prior to the etching step. In any of the preceding embodiments, nucleic acid molecules can be provided in the wells after the etching step. In any of the preceding embodiments, the nucleic acid molecules can be immobilized in the first and second regions after the etching step.

In any of the preceding embodiments, the nucleic acid molecules in the first well and those in the second well can include the same sequence, optionally wherein the nucleic acid molecules are universal among wells on the substrate. In any of the preceding embodiments, the nucleic acid molecules in the first well and those in the second well can have different sequences. In any of the preceding embodiments, any one or more of the nucleic acid molecules can be directly or indirectly attached to the substrate, e.g., via a linker or spacer.

In any of the preceding embodiments, at least 90% of the Round 1 beads can each include a unique Round 1 oligonucleotide, optionally wherein each Round 1 bead can include a unique Round 1 oligonucleotide. In any of the preceding embodiments, at least 90% of the wells can each receive one Round 1 bead, optionally wherein each well can receive one Round 1 bead. In any of the preceding embodiments, the Round 1 oligonucleotides are at least four nucleotides in length.

In any of the preceding embodiments, the released Round 1 oligonucleotides in the first and second wells can include a first Round 1 barcode sequence and a second Round 1 barcode sequence, respectively, wherein the first and second Round 1 barcode sequences can be different. In any of the preceding embodiments, the released Round 1 oligonucleotides in the first and second wells can include a first Round 1 molecular tag and a second Round 1 molecular tag, respectively, wherein the first and second Round 1 molecular tags can be different.

In any of the preceding embodiments, the released Round 1 oligonucleotide can include a sequence that hybridizes to a Round 1 splint which in turn can hybridize to the nucleic acid molecules in a particular well, optionally wherein the released Round 1 oligonucleotide can be ligated to the nucleic acid molecules using the Round 1 splint as template to generate the extended nucleic acid molecules. In any of the preceding embodiments, the sequence that hybridizes to the Round 1 splint can be common between Round 1 oligonucleotides in different wells. In any of the preceding embodiments, the Round 1 splint can be common between different wells. In any of the preceding embodiments, the Round 1 splint can be included in the Round 1 bead and can be released upon disruption of the bead. In some embodiments, the Round 1 splint is not included in the Round 1 bead and is separately delivered to the wells.

In any of the preceding embodiments, the released Round 1 oligonucleotide can include a sequence that hybridizes to a Round 2 splint which in turn can hybridize to a Round 2 oligonucleotide, optionally wherein the Round 2 oligonucleotide can be ligated to the extended nucleic acid molecules using the Round 2 splint as template to generate further extended nucleic acid molecules. In any of the preceding embodiments, the sequence that hybridizes to the Round 2 splint can be common between Round 1 oligonucleotides in different wells. In any of the preceding embodiments, the Round 2 splint may be common between different wells.

In some embodiments, provided are arrays generated by any one of the methods described above.

In some aspects, disclosed herein are methods for providing an array, which can include (a) partitioning a plurality of Round 1 beads into wells on a substrate, wherein the Round 1 bead in a first well and the Round 1 bead in a second well can each include a different Round 1 oligonucleotide; (b) disrupting the Round 1 beads to release the Round 1 oligonucleotides, wherein the released Round 1 oligonucleotides can be attached to nucleic acid molecules in the corresponding well via hybridization and/or ligation to generate extended nucleic acid molecules; (c) partitioning a plurality of Round 2 beads into wells on the substrate, wherein the Round 2 bead in the first well and the Round 2 bead in the second well can each include a different Round 2 oligonucleotide; and (d) disrupting the Round 2 beads to release the Round 2 oligonucleotides, wherein the released Round 2 oligonucleotides can be attached to the extended nucleic acid molecules in the corresponding well via hybridization and/or ligation to generate further extended nucleic acid molecules.

In any of the preceding embodiments, the Round 2 oligonucleotides can be at least four nucleotides in length. In any of the preceding embodiments, the released Round 2 oligonucleotides in the first and second wells can include a first and a second Round 2 barcode sequence, respectively, wherein the first and second Round 2 barcode sequences can be different.

In any of the preceding embodiments, the released Round 2 oligonucleotide can include a sequence that hybridizes to a Round 2 splint which in turn can hybridize to the extended nucleic acid molecules in a particular well, optionally wherein the released Round 2 oligonucleotide can be ligated to the extended nucleic acid molecules using the Round 2 splint as template to generate the further extended nucleic acid molecules. In any of the preceding embodiments, the sequence that hybridizes to the Round 2 splint can be common between Round 2 oligonucleotides in different wells. In any of the preceding embodiments, the Round 2 splint is common between different wells. In any of the preceding embodiments, the Round 2 splint can be included in the Round 2 bead and can be released upon disruption of the Round 2 bead. In some embodiments, the Round 2 splint is not comprised in the Round 2 bead and is separately delivered to the wells.

In any of the preceding embodiments, the released Round 2 oligonucleotide can include a sequence that hybridizes to a Round 3 splint which in turn can hybridize to a Round 3 oligonucleotide, optionally wherein the Round 3 oligonucleotide can be ligated to the further extended nucleic acid molecules using the Round 3 splint as template to generate even further extended nucleic acid molecules. In any of the preceding embodiments, the sequence that hybridizes to the Round 3 splint can be common between Round 2 oligonucleotides in different wells. In any of the preceding embodiments, the Round 3 splint is common between different wells.

In any of the preceding embodiments, the Round 1 oligonucleotide, the Round 2 oligonucleotide, and/or the Round 3 oligonucleotide can each include a unique molecular identifier (UMI) sequence. In any of the preceding embodiments, the Round 1 oligonucleotide, the Round 2 oligonucleotide, and/or the Round 3 oligonucleotide can each include a capture sequence, e.g., poly(dT).

In any of the preceding embodiments, prior to step (a), the substrate can be coated with a photoresist layer, e.g., using spin coating or dipping. In any of the preceding embodiments, the wells are formed by etching a layer of a photoresist on the substrate. In any of the preceding embodiments, the wells are formed in a layer of positive photoresist on the substrate. In any of the preceding embodiments, the method further includes removing the photoresist, leaving the extended nucleic acid molecules, the further extended nucleic acid molecules, or the even further extended nucleic acid molecules immobilized on the substrate.

In any of the preceding embodiments, the Round 1 oligonucleotide, the Round 2 oligonucleotide, and/or the Round 3 oligonucleotide can each include a different capture sequence, wherein each capture sequence can be designed to couple to one or more analytes or proxies thereof. In any of the preceding embodiments, the analyte is a cell surface protein or a secreted analyte. In any of the preceding embodiments, the capture sequence included in the Round 1 oligonucleotides, the Round 2 oligonucleotides, and/or the Round 3 oligonucleotides can be designed to couple to one or more of: a first cell surface protein, a second cell surface protein, a first secreted analyte or a second secreted analyte. In any of the preceding embodiments the Round 1 oligonucleotide can include a capture sequence designed to couple to a first cell surface protein; the Round 2 oligonucleotide can include a capture sequence designed to couple to a second cell surface protein; and/or the Round 3 oligonucleotide can include a capture sequence designed to couple to a first secreted analyte. In any of the preceding embodiments, secreted analyte can be a secreted protein. In any of the preceding embodiments, the cell surface protein includes a proteoglycan and/or a glycoprotein.

In any of the preceding embodiments, the first and/or second wells can be formed by a material enclosing a first region and a second region, respectively, on the substrate. In any of the preceding embodiments, the first region and the second region can be included within a plurality of regions, wherein the plurality of regions can be arranged in a square array.

In some embodiments, provided are arrays generated by any one of the methods described above. In some embodiments, the array can comprise nucleic acid sequences (e.g., barcode sequences) that are generated in a combinatorial fashion, e.g., through cycles and/or rounds of splint hybridization and splinted oligonucleotide ligation as shown in FIG. 4.

In any of the preceding embodiments, the extended nucleic acid molecules, the further extended nucleic acid molecules, or the even further extended nucleic acid molecules, optionally after cleavage from the substrate and/or amplification (e.g., by PCR), can be migrated or can be allowed to migrate into a porous material abutting the wells. In any of the preceding embodiments, the porous material can be a gel and the migration can include electrophoresis. In any of the preceding embodiments, the migration paths in the porous material can be substantially parallel to one another, and the porous material can be divided into subparts along one or more planes intersecting the migration paths, thereby generating copies of the array. In any of the preceding embodiments, the porous material can be divided into subparts along one or more planes that are substantially perpendicular to the mean migration direction.

In some embodiments, provided is a composition comprising a master array and copy arrays, comprising the extended nucleic acid molecules, the further extended nucleic acid molecules, or the even further extended nucleic acid molecules described in any of the preceding embodiments, wherein the method of providing the copy arrays comprises: optionally after cleavage from the substrate and/or amplification (e.g., by PCR), migrating or allowing to migrate the extended nucleic acid molecules, the further extended nucleic acid molecules, or the even further extended nucleic acid molecules into a porous material abutting the wells.

In some aspects, disclosed herein are methods for providing an array, which can include (a) partitioning a plurality of beads into wells on a substrate, wherein the bead in a first well and the bead in a second well can each include a different oligonucleotide; (b) disrupting the plurality of beads to release the oligonucleotides, wherein the released oligonucleotides can be migrated or can be allowed to migrate into a porous material abutting the wells, wherein the migration paths in the porous material can be substantially parallel to one another; and (c) dividing the porous material into subparts along one or more planes intersecting the migration paths, thereby generating copies of the array including the released oligonucleotides. In any of the preceding embodiments, the subparts can be divided along one or more planes, wherein each subpart can be about any one of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 8 mm, or 10 mm in thickness.

In some embodiments, provided are arrays and compositions generated by any one of the methods described above. In some embodiments, the arrays or the compositions can comprise master arrays and/or copy arrays.

In some aspects, disclosed herein are methods for providing a 3D array, which can include a) partitioning a plurality of beads into wells on a substrate, wherein a first bead in a first well can include a plurality of first oligonucleotides of varying lengths which can include the same first barcode sequence, and a second bead in a second well can include a plurality of second oligonucleotides of varying lengths which can include the same second barcode sequence; and (b) disrupting the first and second beads to release the oligonucleotides in the corresponding well, wherein the released oligonucleotides can be migrated or can be allowed to migrate in a direction into a porous material abutting the wells, wherein a position of a particular released oligonucleotide in the direction can correlate with the length of the particular released oligonucleotide, thereby providing in the porous material a 3D-array including the released oligonucleotides, optionally wherein the porous material can be a three-dimensional medium.

In any of the preceding embodiments, the first and second wells can be included within a plurality of N wells, wherein N is an integer and wherein N can have a numerical value greater than any one of 5, 10, 50, 100, 200, 500, 1000, 10000, 20000, 50000, 100000. In any of the preceding embodiments, the plurality of beads can include at least the first bead including the plurality of first oligonucleotides, the second bead including the plurality of second oligonucleotides, and the third bead including the plurality of third oligonucleotide. In any of the preceding embodiments, the oligonucleotides within the plurality of first oligonucleotides and/or the oligonucleotides within the plurality of second oligonucleotides can each have different regimented lengths. In any of the preceding embodiments, the oligonucleotides in each plurality range in length from 10 to 106 nucleotides. In any of the preceding embodiments, length between a shortest and a longest oligonucleotide in the plurality is about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or 50,000 nucleotides. In any of the preceding embodiments, the oligonucleotides in the plurality have regimented lengths of between 1 and 100 nucleotides, 100 and 1000 nucleotides or 1000 and 10,000 nucleotides.

In any of the preceding embodiments, the porous medium can be a three-dimensional medium, wherein the method can further include: (a) migrating the plurality of first oligonucleotides through the three-dimensional medium in a first direction to separate individual oligonucleotides in the first plurality by size and immobilizing the separated oligonucleotides of the first plurality in the medium; and/or (b) migrating the plurality of second oligonucleotides through the three-dimensional medium in a second direction to separate individual oligonucleotides in the second plurality by size and immobilizing the separated oligonucleotides of the second plurality in the medium; and/or (c) migrating the plurality of third oligonucleotides through the three-dimensional medium in a third direction to separate individual oligonucleotides in the first plurality by size and immobilizing the separated oligonucleotides of the third plurality in the medium.

In any of the preceding embodiments, the method further includes analyzing the three-dimensional medium to determine relative locations of the oligonucleotides of the three pluralities. In any of the preceding embodiments, the oligonucleotides in the pluralities can be separated based on size or length of migration through the medium. In any of the preceding embodiments, the medium can include a hydrogel. In any of the preceding embodiments, an electromagnetic field can be applied across the medium to migrate the oligonucleotides through the medium.

In any of the preceding embodiments, the medium can contain cellular RNAs. In any of the preceding embodiments, the oligonucleotides of the first, second and third pluralities include a hybridizing nucleotide sequence capable of identifying the same cellular RNA. In any of the preceding embodiments, the oligonucleotides of the first, second and third pluralities can include a hybridizing nucleotide sequence capable of identifying different cellular RNA. In any of the preceding embodiments, prior to the migrating and the immobilizing of the pluralities of oligonucleotides, the method can include capturing cellular RNAs in the medium using RNA capturing probes and/or production of RNA-derived amplification products. In any of the preceding embodiments, the method can include subjecting the medium containing the separated oligonucleotides to conditions where the hybridizing nucleotide sequence of the separated oligonucleotides hybridizes to the captured cellular RNAs, RNA capturing probes and/or RNA-derived amplification products present in the medium that can have a nucleotide sequence complementary to the hybridizing nucleotide sequence of the oligonucleotides.

In some aspects, disclosed herein is a method, which can include (a) capturing cellular RNAs in a three-dimensional tissue sample using RNA capturing probes and/or sequence-specific amplification of the cellular RNAs; and/or (b) embedding the tissue sample in a conductive polymer. In any of the preceding embodiments, the method can include (c) partitioning a plurality of beads into wells on a substrate, wherein a first bead in a first well can include a plurality of first oligonucleotides of varying lengths which include the same first barcode sequence, a second bead in a second well includes a plurality of second oligonucleotides of varying lengths which includes the same second barcode sequence and a third bead in a third well comprises a plurality of third oligonucleotides of varying lengths which includes the same third barcode sequence; further wherein the first, second and third pluralities of oligonucleotides can encode i) hybridizing nucleotide sequences capable of identifying different RNAs, or ii) nucleotide sequences capable of hybridizing to RNA, RNA capturing probes, or the RNA-derived amplification products that are different; (d) placing the conductive polymer abutting the wells on the substrate; (e) disrupting the first, second and third beads to release the oligonucleotides in the corresponding wells, wherein the released oligonucleotides can be allowed to migrate into the conductive polymer abutting the wells, wherein a position of a particular released oligonucleotide in the direction can correlate with the length of the particular released oligonucleotide; (f) electrophoresing the first plurality of oligonucleotides through a first dimension of the conductive polymer to separate individual oligonucleotides of the first composition by molecular weight, and immobilizing the separated oligonucleotides of the first plurality in the polymer; electrophoresing the second plurality of oligonucleotides through a second dimension of the conductive polymer to separate individual oligonucleotides of the second composition by molecular weight, and immobilizing the separated oligonucleotides of the second plurality in the polymer; electrophoresing the second plurality of oligonucleotides through a third dimension of the conductive polymer to separate individual oligonucleotides of the third composition by molecular weight, and immobilizing the separated oligonucleotides of the third pluralities in the polymer; (g) subjecting the conductive polymer to conditions providing for hybridization of oligonucleotides in the polymer to the captured RNAs, RNA capturing probes and/or RNA-derived amplification products; (h) performing nucleotide sequencing of the hybridized oligonucleotides and captured RNAs, RNA capturing probes and/or RNA-derived amplification products; and (i) integrating sizes of the sequenced oligonucleotides with identity of the captured cellular RNAs to determine relative location of the cellular RNAs in the three-dimensions of the polymer.

In some embodiments, provided are three dimensional arrays (3D-arrays) generated by any one of the methods described herein. In some embodiments, provided are uses of any one of the 3D-arrays generated by methods described herein, wherein the use can comprise performing nucleotide sequencing of the hybridized oligonucleotides and captured RNAs, RNA capturing probes and/or RNA-derived amplification products; and/or integrating sizes of the sequenced oligonucleotides with identity of the captured cellular RNAs to determine relative location of the cellular RNAs in the three-dimensions of the polymer.

### BRIEF DESCRIPTION OF THE DRAWINGS

FIG. 1 shows an exemplary method for generating wells on an array substrate using a photoresist. The top panel shows a cross section of a photoresist layered on top of an array substrate. The arrow indicates that portions of the photoresist are removed to create wells, shown in the bottom panel.

FIGS. 2A-2B show illustrates examples of partitioning and disruption, respectively, of beads in wells on the substrate shown in FIG. 1. FIG. 2A is a schematic illustration of partitioning of beads (circles) comprising oligonucleotides comprising barcodes (BC) 1-3 into wells on the substrate. All the barcodes in a given well (e.g., all the BC1s) are identical to each other and distinct from the other barcodes (e.g., BC1 is distinct from each of BC2 and BC3) in sequence. FIG. 2B illustrates the disruption of beads, and release of oligonucleotides comprising the indicated barcodes into wells on the substrate.

FIG. 3 illustrates an example of hybridization of released oligonucleotides (dashed curved lines) comprising barcodes (e.g., BC1) with nucleic acid molecules (solid curved lines) in the wells from FIG. 2B. The nucleic acid molecules may comprise capture sequences and may be pre-deposited on the substrate.

FIG. 4 illustrates an example of hybridization of splints (shorter dark rectangles in the middle) with released oligonucleotides (top dark rectangles) comprising barcodes and with nucleic acid molecules (bottom shaded rectangles) in a single well shown in cross section. The released oligonucleotides comprising barcodes and the nucleic acid molecules in the wells can be ligated using the splints to form extended nucleic acids in the wells.

FIG. 5 illustrates an example of removal (arrow) of materials enclosing a well with the extended nucleic acids remaining associated with the region corresponding to the well. The top panel shows the starting well, depicted as in FIG. 3. The bottom panel shows that the sides of the well have been removed.

FIGS. 6A-6B show schematics of exemplary methods of array construction. FIG. 6A illustrates a method of generating an array by multiple rounds of bead partitioning to release oligonucleotides comprising parts of barcodes. FIG. 6B is a graph showing parts of oligonucleotides installed in sequential rounds each comprising multiple cycles of bead-assisted splint hybridization and ligation.

FIG. 7 is illustrates an example of migration (arrows) of nucleic acid molecules comprising barcodes (BC1, BC2, and BC3) from the array (bottom left) into an abutting porous material (top left), followed by removal of porous material comprising the migrated nucleic acid molecules from the substrate (top right), and separation of the porous materials into subparts (bottom right, wherein each row is a subpart).

FIG. 8 illustrates an example population of oligonucleotides (with each row representing one oligonucleotide) of different lengths, containing variable lengths of nucleic acid sequences (e.g., known sequences, shown as shaded rectangles) and the same barcode sequence (shown as curves).

FIG. 9 is illustrates an example of migration (arrows pointing up) of nucleic acid molecules comprising barcodes 1-3 (BC1, BC2, and BC3, respectively) from the array into an abutting porous material (top panel), followed by removal (arrow pointing down) of porous material comprising the migrated nucleic acid molecules from the substrate (bottom panel).

FIG. 10 is a schematic illustration of an exemplary method of using a barcoded bead array and mapping optical measurements of cells taken on the array to the corresponding barcode(s). A starting barcoded array is shown in the top panel, with beads shown as circles inside cross-sections of wells on an array substrate. Beads that are shaded with different patterns comprise different barcode sequences. In the middle panel, reagents are added to the wells, shown as an overlay of dashes. In the bottom panel, cells (small gray circles) are added at limiting dilution (e.g., such that each well contains only 0 or 1 cells). The cells are then imaged and barcoded libraries are made and sequenced.

FIG. 11 is a schematic illustration of an exemplary method of using a molecular array comprising barcodes in a known spatial pattern and mapping optical measurements of cells taken on the array to the corresponding barcode(s). A starting barcoded array is shown in the top panel, with barcodes shown as shorter lines inside cross-sections of wells on an array substrate. The barcodes in different wells comprise different sequences. In the middle panel, reagents are added to the wells, shown as an overlay of dashes. In the bottom panel, cells (small gray circles) are added, limiting dilution (e.g., such that each well contains only 0 or 1 cells). The cells are then imaged and barcoded libraries are made and sequenced.

### DETAILED DESCRIPTION

The practice of the techniques described herein may employ, unless otherwise indicated, conventional techniques and descriptions of organic chemistry, polymer technology, molecular biology (comprising recombinant techniques), cell biology, biochemistry, and sequencing technology, which are within the skill of those who practice in the art. Such conventional techniques comprise polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3rd Ed., W.H. Freeman Pub., New York, N.Y.; and Berg et al. (2002)Biochemistry, 5^{th} Ed., W. H. Freeman Pub., New York, N.Y., the contents of all of which are herein incorporated in their entireties by reference for all purposes.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

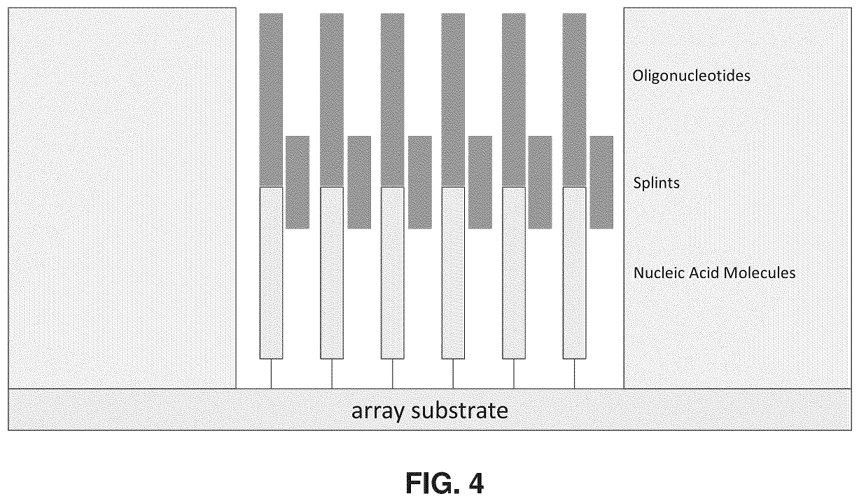

Oligonucleotide arrays for spatial transcriptomics may be made by mechanical spotting, bead arrays, and/or in situ base-by-base synthesis of the oligonucleotides. In some cases, mechanical spotting is ideal for larger spot sizes (e.g., 30 microns in diameter or greater), since fully elaborated oligos (e.g., with a desired combination and diversity of barcodes) can be spotted in a known position with high purity and fidelity. However, methods to decrease spot sizes or features at or below 10 microns (e.g., single cell scale resolution) in diameter with sufficient throughput are lacking. In some aspects, bead arrays offer a way to increase feature density. For example, barcodes are generated by first attaching an oligonucleotide to all beads and then performing multiple rounds of split-pool ligations to generate barcodes combinatorially. In some aspects, provided are methods to generate arrays using directed bead delivery to deposit oligonucleotides and generate extended nucleic acid molecules. Also provided are ways of generating arrays comprising nucleic acid sequences (e.g., barcode sequences that are generated in a combinatorial fashion, e.g., through cycles and/or rounds of splint hybridization and splinted oligonucleotide ligation as shown in FIG. 4), master and copy arrays as well as three-dimensional arrays, as well as compositions and applications thereof.

In some embodiments, a large diversity of barcodes, comprised within oligonucleotides, can be created in molecules on the substrate via sequential rounds of partitioning of beads comprising oligonucleotides into spatially predefined regions. Further provided in some aspects are further applications of such patterned arrays, such as generation of masters arrays and migration of extended nucleic acid molecules to form copy arrays. In some aspects, provided are methods of partitioning of beads comprising oligonucleotides into spatially predefined regions (including but not limited to wells), to generate unique DNA sequences in spatial positions in the array, followed by multi-directional migration of such DNA sequences into a porous material to form 3-dimensional arrays with large diversity of barcodes. Also provided are compositions of such arrays and uses thereof, such as interrogating tissue RNAs embedded into porous material via tissue-gel exchange.

### II. Bead Delivery and Molecular Arrays

A. Methods of Generating Molecular Arrays

In some embodiments, a substrate comprising an array of molecules is provided, e.g., in the form of a lawn of polymers (e.g., oligonucleotides) on the substrate in a pattern. Examples of polymers on an array may include, but are not limited to, nucleic acids, peptides, phospholipids, polysaccharides, heteromacromolecules in which a known drug is covalently bound to any of the above, polyurethanes, polyesters, polycarbonates, polyureas, polyamides, polyethyleneimines, polyarylene sulfides, polysiloxanes, polyimides, and polyacetates. The molecules occupying different features of an array typically differ from one another, although some redundancy in which the same polymer occupies multiple features can be useful as a control. For example, in a nucleic acid array, the nucleic acid molecules within the same feature are typically the same, whereas nucleic acid molecules occupying different features are mostly different from one another.

In some aspects, provided herein is a method of patterning a surface in situ for producing an array on the surface, for example, by partitioning of beads comprising oligonucleotides into spatially predefined regions (including but not limited to wells), to generate unique DNA sequences in spatial positions in the array.

In some aspects, provided is a method of generating an array, comprising: (a) partitioning a plurality of beads into wells on a substrate, wherein the bead in each well each comprises a different oligonucleotide; and (b) disrupting the beads to release the oligonucleotides, wherein the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the oligonucleotides comprised in the beads comprise barcode sequences. In some embodiments, the in situ method comprises one or more steps of partitioning pluralities of beads that enable barcodes to be generated combinatorially, for example, in as few as three rounds of assembly. The process can be repeated N cycles (each cycle for one or more partitions and disruptions of beads) for Round 1 until all desired wells have received partitioned beads and/or release of oligonucleotides following disruptions of beads. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round M barcode (which may be the same or different for molecules in any two given features) to each of the growing oligonucleotides in the features.

In some embodiments, provided is a method for generating an array, comprising: (a) partitioning a plurality of Round 1 beads into wells on a substrate, wherein the Round 1 bead in a first well and the Round 1 bead in a second well each comprises a different Round 1 oligonucleotide; and (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules, thereby providing on the substrate an array comprising the extended nucleic acid molecules.

In some embodiments, the method comprises partitioning beads into a plurality of wells. In some embodiments, the method comprises partitioning beads into one or more wells. In some embodiments, the one or more wells are formed by a material enclosing one or more corresponding regions on the substrate. In some embodiments, the one or more wells are formed by etching a layer of the material on the substrate. In some embodiments, the nucleic acid molecules are immobilized on the substrate prior to the etching step. In some embodiments, the nucleic acids are immobilized in one or more regions prior to the etching step. In some embodiments, the nucleic acids are immobilized in the first and second regions prior to the etching step. In some embodiments, the nucleic acids are provided in one or more wells after the etching step. In some embodiments, the nucleic acids are provided in the first and second wells after the etching step. In some embodiments, the nucleic acids are immobilized in one or more regions after the etching step. In some embodiments, the nucleic acids are immobilized in the first and second regions after the etching step.

In some embodiments, the method comprises partitioning beads into at least the first well and the second well. In some embodiments, the first and/or second wells are formed by a material enclosing a first region and a second region, respectively, on the substrate. In some embodiments, the first and/or second wells are formed by etching a layer of the material on the substrate on the substrate. In some embodiments, the one or more wells are formed by etching a layer of the material on the substrate

In some embodiments, the nucleic acid molecules in the one or more wells comprise the same sequence. In some embodiments, the nucleic acid molecules in the first well and the nucleic acid molecules in the second well comprise the same sequence. In some embodiments, the nucleic acid molecules are universal among wells on the substrate.

In some embodiments, the nucleic acid molecules in each of the plurality of wells are different in sequences. In some embodiments, the nucleic acid molecules in the first well and those in the second well are different in sequences. In some embodiments, the nucleic acid in at least about any one of: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the plurality of wells are unique in sequences. In some embodiments, the nucleic acid in each of the plurality of wells are unique in sequences. In some embodiments, the nucleic acid molecules in two wells of a plurality of wells comprise sequences that are at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical. In some embodiments, nucleic acid molecules in two wells of a plurality of wells comprise sequences that are no more than about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round M beads each comprises a unique Round M oligonucleotide, wherein M is an integer that is 1 or greater. In some embodiments, at least about 90% of Round M beads each comprises a unique Round M oligonucleotide, wherein M is an integer that is 1 or greater. In some embodiments, each Round M bead comprises a unique Round M oligonucleotide, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 1 beads each comprises a unique Round 1 oligonucleotide. In some embodiments, at least about 90% of Round 1 beads each comprise a unique Round 1 oligonucleotide. In some embodiments, each Round 1 bead comprises a unique Round 1 oligonucleotide.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives only one Round M bead, wherein M is an integer that is 1 or greater. In some embodiments, at least about 90% of wells each receives only one Round M bead, wherein M is an integer that is 1 or greater. In some embodiments, each well receives only one Round M bead, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives only one Round 1 bead. In some embodiments, at least about 90% of wells each receives only one Round 1 bead. In some embodiments, each well receives only one Round 1 bead.

In some embodiments, the Round M oligonucleotides are at least about any one of 3, 4, 5, 6, 7, 8, 9, 10, 12, 14, 16, 18, 20, 25, 30, 35, 40, 45, or 50 nucleotides in length, wherein M is an integer that is 1 or greater. In some embodiments, the Round M oligonucleotides are at least about 4 nucleotides in length, wherein M is an integer that is 1 or greater. In some embodiments, the Round 1 oligonucleotides are at least about any one of 3, 4, 5, 6, 7, 8, 9, 10, 12, 14, 16, 18, 20, 25, 30, 35, 40, 45, or 50 nucleotides in length. In some embodiments, the Round 1 oligonucleotides are at least about 4 nucleotides in length.

In some embodiments, the released Round 1 oligonucleotides in the plurality of wells comprise a plurality of corresponding Round 1 barcode sequences, respectively, wherein each of the plurality of Round 1 barcode sequences are different. In some embodiments, the released Round 1 oligonucleotides in the first and second wells comprise a first Round 1 barcode sequence and a second Round 1 barcode sequence, respectively, wherein the first and second Round 1 barcode sequences are different.

In some embodiments, the released Round 1 oligonucleotides in the plurality of wells comprise a plurality of corresponding Round 1 molecular tags, respectively, wherein each of the plurality of Round 1 molecular tags are different. In some embodiments, the released Round 1 oligonucleotides in the first and second wells comprise a first Round 1 molecular tag and a second Round 1 molecular tag, respectively, wherein the first and second Round 1 molecular tags are different.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

In some embodiments, the released Round 1 oligonucleotide comprises a sequence that hybridizes to a Round 1 splint which in turn hybridizes to the nucleic acid molecules in a particular well. In some embodiments, the released Round 1 oligonucleotide is ligated to the nucleic acid molecules using the Round 1 splint as template to generate the extended nucleic acid molecules.

In some embodiments, the sequence that hybridizes to the Round 1 splint is common between Round 1 oligonucleotides in different wells. In some embodiments, the Round 1 splint is comprised in the Round 1 bead and released upon disruption of the bead. In some embodiments, the Round 1 splint is not comprised in the Round 1 bead and is separately delivered to the wells.

In some embodiments, the method is repeated for M rounds, wherein M is an integer that is 1 or greater. In some embodiments, the sequence that hybridizes to the Round M splint is common between Round M oligonucleotides in different wells. In some embodiments, the Round M splint is comprised in the Round M bead and released upon disruption of the bead. In some embodiments, the Round M splint is not comprised in the Round M bead and is separately delivered to the wells.

In some embodiments, wherein the method is repeated for M rounds, wherein the released Round M oligonucleotide comprises a sequence that hybridizes to a Round M+1 splint which in turn hybridizes to a Round M+1 oligonucleotide, optionally wherein the Round M+1 oligonucleotide is ligated to the extended nucleic acid molecules using the Round M+1 splint as template to generate further extended nucleic acid molecules. In some embodiments, the sequence that hybridizes to the Round M+1 splint is common between Round M oligonucleotides in different wells. In some embodiments, the Round M+1 splint is common between different wells.

In some embodiments, the released Round 1 oligonucleotide comprises a sequence that hybridizes to a Round 2 splint which in turn hybridizes to a Round 2 oligonucleotide, optionally wherein the Round 2 oligonucleotide is ligated to the extended nucleic acid molecules using the Round 2 splint as template to generate further extended nucleic acid molecules. In some embodiments, the sequence that hybridizes to the Round 2 splint is common between Round 1 oligonucleotides in different wells. In some embodiments, the Round 2 splint is common between different wells

In some embodiments, the hybridization region between the Round 1 splint and nucleic acid molecule is at least 3, 4, 5, 6, 7, 8, 9, 10 bp or more than 10 bp, and/or wherein the hybridization region between the Round 1 splint and the Round 1 oligonucleotide is at least 3, 4, 5, 6, 7, 8, 9, 10 bp or more than 10 bp. In some embodiments, the hybridization region between the Round 2 splint and the Round 1 oligonucleotide is at least 3, 4, 5, 6, 7, 8, 9, 10 bp or more than 10 bp, and/or wherein the hybridization region between the Round 2 splint and the Round 2 oligonucleotide is at least 3, 4, 5, 6, 7, 8, 9, 10 bp or more than 10 bp.

In some embodiments, the hybridization region between the Round M+1 splint and the Round M oligonucleotide is at least 3, 4, 5, 6, 7, 8, 9, 10 bp or more than 10 bp, and/or wherein the hybridization region between the Round M+1 splint and the Round M+1 oligonucleotide is at least 3, 4, 5, 6, 7, 8, 9, 10 bp or more than 10 bp, wherein M is an integer that is 1 or greater.

In some embodiments, hybridization to the Round 1 splint brings the terminal nucleotides of the Round 1 oligonucleotides and the nucleic acid molecules immediately next to each other, and the ligation does not require gap-filling, thereby generating a plurality of first extended nucleic acid molecules; and/or hybridization to the Round 2 splint brings the terminal nucleotides of the Round 2 oligonucleotide molecules and the first extended nucleic acid molecules immediately next to each other, and the ligation does not require gap-filling, thereby generating the second extended nucleic acid molecules. In some embodiments, hybridization to the Round M+1 splint brings the terminal nucleotides of the Round M oligonucleotides and the Mth extended nucleic acid molecules immediately next to each other, and the ligation does not require gap-filling, thereby generating a plurality of (M+1)th extended nucleic acid molecules; where M is an integer that is 1 or greater.

In some embodiments, hybridization to the Round 1 splint brings the terminal nucleotides of the Round 1 oligonucleotides and the nucleic acid molecules next to each other and separated by one or more nucleotides, and the ligation is preceded by gap-filling, thereby generating the first extended nucleic acid molecules; and/or hybridization to the Round 2 splint brings the terminal nucleotides of the Round 2 oligonucleotides and the first extended nucleic acid molecules next to each other and separated by one or more nucleotides, and the ligation is preceded by gap-filling, thereby generating the second extended nucleic acid molecules. In some embodiments, hybridization to the Round M+1 splint brings the terminal nucleotides of the Round M oligonucleotides and the Mth extended nucleic acid molecules next to each other and separated by one or more nucleotides, and the ligation is preceded by gap-filling, thereby generating a plurality of (M+1)th extended nucleic acid molecules; where M is an integer that is 1 or greater.

In some aspects, provided herein is a method of patterning a surface in situ for producing an array on the surface, for example, by one or more rounds of partitioning of beads comprising oligonucleotides into spatially predefined regions (including but not limited to wells), to generate unique DNA sequences (e.g., barcode sequences) combinatorially (e.g., through sequential cycles and/or rounds of nucleic acid hybridization/ligation such as shown in FIG. 4) in spatial positions in the array.

In some aspects, provided is a method of generating an array, comprising: (a) partitioning a plurality of beads into wells on a substrate, wherein the bead in each well each comprises a different oligonucleotide; and (b) disrupting the beads to release the oligonucleotides, wherein the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the oligonucleotides comprised in the beads comprise barcode sequences. In some embodiments, the in situ method comprises one or more steps of partitioning pluralities of beads that enable barcodes to be generated combinatorially, for example, in as few as three rounds of assembly. The process can be repeated N cycles (each cycle for one or more partitions and disruptions of beads) for Round 1 until all desired wells have received partitioned beads and/or release of oligonucleotides following disruptions of beads. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round M barcode (which may be the same or different for molecules in any two given features) to each of the growing oligonucleotides in the well.

In some embodiments, provided is a method for generating an array, comprising: (a) partitioning a plurality of Round 1 beads into a plurality of wells on a substrate, wherein the Round 1 beads in each of the plurality of wells each comprises a different Round 1 oligonucleotide; (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules, (c) partitioning a plurality of Round 2 beads into the plurality of wells on the substrate, wherein Round 2 beads in each of the plurality of wells each comprises a different Round 2 oligonucleotide; and (d) disrupting the Round 2 beads to release the Round 2 oligonucleotides, wherein the released Round 2 oligonucleotides are attached to the extended nucleic acid molecules in the corresponding well via hybridization and/or ligation to generate further extended nucleic acid molecules (or the second extended nucleic acid molecules). In some embodiments, the method further comprises the step of: (e) partitioning a plurality of Round M beads into wells on a substrate, wherein the Round M beads in each of the plurality of wells each comprises a different Round M oligonucleotide; (f) disrupting the Round M beads to release the Round M oligonucleotides, wherein the released Round M oligonucleotides are attached to (M−1)th extended nucleic acid molecules in the corresponding well via hybridization and/or ligation to generate (M)th extended nucleic acid molecules; (g) partitioning a plurality of Round M+1 beads into wells on a substrate, wherein the Round M+1 beads in each of the plurality of wells each comprises a different Round M+1 oligonucleotide; (f) disrupting the Round M+1 beads to release the Round M+1 oligonucleotides, wherein the released Round M+1 oligonucleotides are attached to (M)th extended nucleic acid molecules in the corresponding well via hybridization and/or ligation to generate (M+1)th extended nucleic acid molecules, wherein M is an integer that is greater than 2, optionally wherein the step of (e), (f), (g), and (h) are repeated N times, wherein M is incremented by 2 in each repetition, wherein N is an integer that is about 2, 3, 4, 5, 6, 7, 8, 9, 10, 15, 20, 25, 30, 35, 40, 50, or greater.

In some embodiments, provided is a method for generating an array, comprising: (a) partitioning a plurality of Round 1 beads into wells on a substrate, wherein the Round 1 bead in a first well and the Round 1 bead in a second well each comprises a different Round 1 oligonucleotide; (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules (or the first extended molecules), (c) partitioning a plurality of Round 2 beads into wells on the substrate, wherein the Round 2 bead in the first well and the Round 2 bead in the second well each comprises a different Round 2 oligonucleotide; and (d) disrupting the Round 2 beads to release the Round 2 oligonucleotides, wherein the released Round 2 oligonucleotides are attached to the extended nucleic acid molecules in the corresponding well via hybridization and/or ligation to generate further extended nucleic acid molecules (or the second extended nucleic acid molecules). In some embodiments, wherein the method can be repeated for M rounds, wherein M is an integer that is greater than 2. In some embodiments, the method further comprises the step of: (e) partitioning a plurality of Round M beads into wells on a substrate, wherein the Round M bead in a first well and the Round M bead in a second well each comprises a different Round M oligonucleotide; (f) disrupting the Round M beads to release the Round M oligonucleotides, wherein the released Round M oligonucleotides are attached to (M−1)th extended nucleic acid molecules in the corresponding well via hybridization and/or ligation to generate (M)th extended nucleic acid molecules; (g) partitioning a plurality of Round M+1 beads into wells on a substrate, wherein the Round M+1 bead in a first well and the Round M+1 bead in a second well each comprises a different Round M+1 oligonucleotide; (f) disrupting the Round M+1 beads to release the Round M+1 oligonucleotides, wherein the released Round M+1 oligonucleotides are attached to (M)th extended nucleic acid molecules in the corresponding well via hybridization and/or ligation to generate (M+1)th extended nucleic acid molecules, wherein M is an integer that is greater than 2, optionally wherein the step of (e), (f), (g), and (h) are repeated N times, wherein M is incremented by 2 in each repetition, wherein N is 2, 3, 4, 5, 6, 7, 8, 9, 10, 15, 20, 25, 30, 35, 40, 50, or higher.

In some embodiments, the method comprises partitioning beads into a plurality of wells. In some embodiments, the method comprises partitioning beads into one or more wells. In some embodiments, the one or more wells are formed by a material enclosing one or more corresponding regions on the substrate. In some embodiments, the one or more wells are formed by etching a layer of the material on the substrate. In some embodiments, the nucleic acid molecules are immobilized on the substrate prior to the etching step. In some embodiments, the nucleic acids are immobilized in one or more regions prior to the etching step. In some embodiments, the nucleic acids are immobilized in the first and second regions prior to the etching step. In some embodiments, the nucleic acids are provided in one or more wells after the etching step. In some embodiments, the nucleic acids are provided in the first and second wells after the etching step. In some embodiments, the nucleic acids are immobilized in one or more regions after the etching step. In some embodiments, the nucleic acids are immobilized in the first and second regions after the etching step.

In some embodiments, the method comprises partitioning beads into at least the first well and the second well. In some embodiments, the first and/or second wells are formed by a material enclosing a first region and a second region, respectively, on the substrate. In some embodiments, the first and/or second wells are formed by etching a layer of the material on the substrate on the substrate. In some embodiments, the one or more wells are formed by etching a layer of the material on the substrate

In some embodiments, the nucleic acid molecules in the one or more wells comprise the same sequence. In some embodiments, the nucleic acid molecules immobilized in the first well and the nucleic acid molecules in the second well comprise the same sequence. In some embodiments, the nucleic acid molecules are universal among wells on the substrate.

In some embodiments, the nucleic acid molecules in each of the plurality of wells are different in sequences. In some embodiments, the nucleic acid molecules in the first well and those in the second well are different in sequences. In some embodiments, the nucleic acid molecules in at least about any one of: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the plurality of wells are unique in sequences. In some embodiments, the nucleic acid molecules each of the plurality of wells are unique in sequences. In some embodiments, the nucleic acid molecules in two wells of a plurality of wells comprise sequences that are at least about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical. In some embodiments, nucleic acid molecules in two wells of a plurality of wells comprise sequences that are no more than about 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% identical.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round M beads each comprises a unique Round M oligonucleotide, wherein M is an integer that is 1 or greater. In some embodiments, at least about 90% of Round M beads each comprises a unique Round M oligonucleotide, wherein M is an integer that is 1 or greater. In some embodiments, each Round M bead comprises a unique Round M oligonucleotide, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 1 beads each comprises a unique Round 1 oligonucleotide. In some embodiments, at least about 90% of Round 1 beads each comprise a unique Round 1 oligonucleotide. In some embodiments, each Round 1 bead comprises a unique Round 1 oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 2 beads each comprises a unique Round 2 oligonucleotide. In some embodiments, at least about 90% of Round 2 beads each comprise a unique Round 2 oligonucleotide. In some embodiments, each Round 2 bead comprises a unique Round 2 oligonucleotide.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives no more than one Round M bead, wherein M is an integer that is 1 or greater. In some embodiments, at least about 90% of wells each receives no more than one Round M bead, wherein M is an integer that is 1 or greater. In some embodiments, each well receives no more than one Round M bead, wherein M is an integer that is 1 or greater. In some embodiments, each Round M bead comprises a unique Round M oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives no more than one Round 1 bead. In some embodiments, at least about 90% of wells each receives no more than one Round 1 bead. In some embodiments, each well receives no more than one Round 1 bead. In some embodiments, each Round 1 bead comprises a unique Round 1 oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives no more than one Round 2 bead. In some embodiments, at least about 90% of wells each receives no more than one Round 2 bead. In some embodiments, each well receives no more than one Round 2 bead. In some embodiments, each Round 2 bead comprises a unique Round 2 oligonucleotide.

In some embodiments, the Round M oligonucleotides are at least about any one 3, 4, 5, 6, 7, 8, 9, 10, 12, 14, 16, 18, 20, 25, 30, 35, 40, 45, or 50 nucleotides in length, wherein M is an integer that is 1 or greater. In some embodiments, the Round M oligonucleotides are at least about 4 nucleotides in length, wherein M is an integer that is 1 or greater. In some embodiments, the Round 1 oligonucleotides are at least about any one 3, 4, 5, 6, 7, 8, 9, 10, 12, 14, 16, 18, 20, 25, 30, 35, 40, 45, or 50 nucleotides in length. In some embodiments, the Round 1 oligonucleotides are at least about 4 nucleotides in length. In some embodiments, the Round 2 oligonucleotides are at least about any one 3, 4, 5, 6, 7, 8, 9, 10, 12, 14, 16, 18, 20, 25, 30, 35, 40, 45, or 50 nucleotides in length. In some embodiments, the Round 2 oligonucleotides are at least about 4 nucleotides in length.

In some embodiments, the released Round 1 oligonucleotides in the plurality of wells comprise a plurality of corresponding Round 1 barcode sequences, respectively, wherein each of the plurality of Round 1 barcode sequences are different. In some embodiments, the released Round 1 oligonucleotides in the first and second wells comprise a first Round 1 barcode sequence and a second Round 1 barcode sequence, respectively, wherein the first and second Round 1 barcode sequences are different. In some embodiments, the released Round 2 oligonucleotides in the plurality of wells comprise a plurality of corresponding Round 2 barcode sequences, respectively, wherein each of the plurality of Round 2 barcode sequences are different. In some embodiments, the released Round 2 oligonucleotides in the first and second wells comprise a first Round 2 barcode sequence and a second Round 2 barcode sequence, respectively, wherein the first and second Round 2 barcode sequences are different. In some embodiments, the released Round M oligonucleotides in the plurality of wells comprise a plurality of corresponding Round M barcode sequences, respectively, wherein each of the plurality of Round M barcode sequences are different. In some embodiments, the released Round M oligonucleotides in the first and second wells comprise a first Round M barcode sequence and a second Round M barcode sequence, respectively, wherein the first and second Round M barcode sequences are different, wherein M is an integer that is 1 or greater.

In some embodiments, the released Round 1 oligonucleotides in the plurality of wells comprise a plurality of corresponding Round 1 molecular tags, respectively, wherein each of the plurality of Round 1 molecular tags are different. In some embodiments, the released Round 1 oligonucleotides in the first and second wells comprise a first Round 1 molecular tag and a second Round 1 molecular tag, respectively, wherein the first and second Round 1 molecular tags are different. In some embodiments, the released Round 2 oligonucleotides in the plurality of wells comprise a plurality of corresponding Round 2 molecular tags, respectively, wherein each of the plurality of Round 2 molecular tags are different. In some embodiments, the released Round 2 oligonucleotides in the first and second wells comprise a first Round 2 molecular tag and a second Round 2 molecular tag, respectively, wherein the first and second Round 2 molecular tags are different. In some embodiments, the released Round M oligonucleotides in the plurality of wells comprise a plurality of corresponding Round M molecular tags, respectively, wherein each of the plurality of Round M molecular tags are different. In some embodiments, the released Round M oligonucleotides in the first and second wells comprise a first Round M molecular tag and a second Round M molecular tag, respectively, wherein the first and second Round M molecular tags are different, wherein M is an integer that is 1 or greater.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

In some embodiments, the released Round 1 oligonucleotide comprises a sequence that hybridizes to a Round 1 splint which in turn hybridizes to the nucleic acid molecules in a particular well. In some embodiments, the released Round 1 oligonucleotide is ligated to the nucleic acid molecules using the Round 1 splint as template to generate the (first) extended nucleic acid molecules. In some embodiments, the released Round 2 oligonucleotide comprises a sequence that hybridizes to a Round 2 splint which in turn hybridizes to the (first) extended nucleic acid molecules in a particular well. In some embodiments, the released Round 2 oligonucleotide is ligated to the (first) extended nucleic acid molecules using the Round 2 splint as template to generate the further extended nucleic acid molecules (second extended nucleic acid molecules). In some embodiments, the released Round M oligonucleotide comprises a sequence that hybridizes to a Round M splint which in turn hybridizes to the (M−1)th extended nucleic acid molecules in a particular well. In some embodiments, the released Round M oligonucleotide is ligated to the (M−1)th extended nucleic acid molecules using the Round M splint as template to generate the (M)th extended nucleic acid molecules, wherein M is an integer greater than 1.

In some embodiments, the sequence that hybridizes to the Round 1 splint is common between Round 1 oligonucleotides in different wells. In some embodiments, the Round 1 splint is comprised in the Round 1 bead and released upon disruption of the bead. In some embodiments, the Round 1 splint is not comprised in the Round 1 bead and is separately delivered to the wells. In some embodiments, the sequence that hybridizes to the Round 2 splint is common between Round 2 oligonucleotides in different wells. In some embodiments, the Round 2 splint is comprised in the Round 2 bead and released upon disruption of the bead. In some embodiments, the Round 2 splint is not comprised in the Round 2 bead and is separately delivered to the wells. In some embodiments, the sequence that hybridizes to the Round 3 splint is common between Round 3 oligonucleotides in different wells. In some embodiments, the Round 3 splint is comprised in the Round 3 bead and released upon disruption of the bead. In some embodiments, the Round 3 splint is not comprised in the Round 3 bead and is separately delivered to the wells.

In some embodiments, the method is repeated for M rounds, wherein M is an integer that is 1 or greater. In some embodiments, the sequence that hybridizes to the Round M splint is common between Round M oligonucleotides in different wells. In some embodiments, the Round M splint is comprised in the Round M bead and released upon disruption of the bead. In some embodiments, the Round M splint is not comprised in the Round M bead and is separately delivered to the wells.

In some embodiments, wherein the method is repeated for M rounds, wherein the released Round M oligonucleotide comprises a sequence that hybridizes to a Round M+1 splint which in turn hybridizes to a Round M+1 oligonucleotide, optionally wherein the Round M+1 oligonucleotide is ligated to the (M)th extended nucleic acid molecules using the Round M+1 splint as template to generate (M+1)th extended nucleic acid molecules. In some embodiments, the sequence that hybridizes to the Round M+1 splint is common between Round M+1 oligonucleotides in different wells. In some embodiments, the Round M+1 splint is common between different wells.

In some embodiments, the released Round 1 oligonucleotide comprises a sequence that hybridizes to a Round 2 splint which in turn hybridizes to a Round 2 oligonucleotide, optionally wherein the Round 2 oligonucleotide is ligated to the (first) extended nucleic acid molecules using the Round 2 splint as template to generate further extended nucleic acid molecules (e.g. second extended nucleic acid molecules). In some embodiments, the sequence that hybridizes to the Round 2 splint is common between Round 1 oligonucleotides in different wells. In some embodiments, the Round 2 splint is common between different wells.

In some embodiments, the released Round 2 oligonucleotide comprises a sequence that hybridizes to a Round 3 splint which in turn hybridizes to a Round 3 oligonucleotide, optionally wherein the Round 3 oligonucleotide is ligated to the further extended nucleic acid molecules (e.g. second extended nucleic acid molecules) using the Round 3 splint as template to generate even further extended nucleic acid molecules (e.g. third extended nucleic acid molecules). In some embodiments, the sequence that hybridizes to the Round 3 splint is common between Round 2 oligonucleotides in different wells. In some embodiments, the Round 3 splint is common between different wells.

In some embodiments, the hybridization region between the Round 1 splint and nucleic acid molecule is at least 3, 4, 5, 6, 7, 8, 9, 10 bp or more than 10 bp, and/or wherein the hybridization region between the Round 1 splint and the Round 1 oligonucleotide is at least 3, 4, 5, 6, 7, 8, 9, 10 bp or more than 10 bp. In some embodiments, the hybridization region between the Round 2 splint and the Round 1 oligonucleotide is at least 3, 4, 5, 6, 7, 8, 9, 10 bp or more than 10 bp, and/or wherein the hybridization region between the Round 2 splint and the Round 2 oligonucleotide is at least 3, 4, 5, 6, 7, 8, 9, 10 bp or more than 10 bp. In some embodiments, the hybridization region between the Round 3 splint and the Round 2 oligonucleotide is at least 3, 4, 5, 6, 7, 8, 9, 10 bp or more than 10 bp, and/or wherein the hybridization region between the Round 3 splint and the Round 3 oligonucleotide is at least 3, 4, 5, 6, 7, 8, 9, 10 bp or more than 10 bp.

In some embodiments, the hybridization region between the Round M+1 splint and the Round M oligonucleotide is at least 3, 4, 5, 6, 7, 8, 9, 10 bp or more than 10 bp, and/or wherein the hybridization region between the Round M+1 splint and the Round M+1 oligonucleotide is at least 3, 4, 5, 6, 7, 8, 9, 10 bp or more than 10 bp, wherein M is an integer that is 1 or greater.

In some embodiments, hybridization to the Round 1 splint brings the terminal nucleotides of the Round 1 oligonucleotides and the nucleic acid molecules immediately next to each other, and the ligation does not require gap-filling, thereby generating a plurality of (first) extended nucleic acid molecules; and/or hybridization to the Round 2 splint brings the terminal nucleotides of the Round 2 oligonucleotide molecules and the (first) extended nucleic acid molecules immediately next to each other, and the ligation does not require gap-filling, thereby generating the further extended nucleic acid molecules (second extended nucleic acid molecules); and/or hybridization to the Round 3 splint brings the terminal nucleotides of the Round 3 oligonucleotide molecules and the further extended nucleic acid molecules (second extended nucleic acid molecules) immediately next to each other, and the ligation does not require gap-filling, thereby generating the even further extended nucleic acid molecules (third extended nucleic acid molecules). In some embodiments, hybridization to the Round M+1 splint brings the terminal nucleotides of the Round M oligonucleotides and the Mth extended nucleic acid molecules immediately next to each other, and the ligation does not require gap-filling, thereby generating a plurality of (M+1)th extended nucleic acid molecules; where M is an integer that is 1 or greater.

In some embodiments, hybridization to the Round 1 splint brings the terminal nucleotides of the Round 1 oligonucleotides and the nucleic acid molecules next to each other and separated by one or more nucleotides, and the ligation is preceded by gap-filling, thereby generating the first extended nucleic acid molecules; and/or hybridization to the Round 2 splint brings the terminal nucleotides of the Round 2 oligonucleotides and the first extended nucleic acid molecules next to each other and separated by one or more nucleotides, and the ligation is preceded by gap-filling, thereby generating the second extended nucleic acid molecules; and/or hybridization to the Round 3 splint brings the terminal nucleotides of the Round 3 oligonucleotides and the second extended nucleic acid molecules next to each other and separated by one or more nucleotides, and the ligation is preceded by gap-filling, thereby generating the third extended nucleic acid molecules. In some embodiments, hybridization to the Round M+1 splint brings the terminal nucleotides of the Round M oligonucleotides and the Mth extended nucleic acid molecules next to each other and separated by one or more nucleotides, and the ligation is preceded by gap-filling, thereby generating a plurality of (M+1)th extended nucleic acid molecules; wherein M is an integer that is 1 or greater.

In some embodiments, the Round 1 oligonucleotide, the Round 2 oligonucleotide, and/or the Round 3 oligonucleotide each comprises a unique molecular identifier (UMI) sequence. In some embodiments, each of the oligonucleotides of M rounds (e.g. Rounds 1, 2, 3, 4, 5 . . . up to M) comprises a unique molecular identifier (UMI) sequence, wherein M is an integer that is greater than 1.

In some embodiments, the Round 1 oligonucleotide, the Round 2 oligonucleotide, and/or the Round 3 oligonucleotide each comprises a capture sequence. In some embodiments, each of the oligonucleotides of M rounds (e.g. Rounds 1, 2, 3, 4, 5 . . . up to M) comprises a capture sequence, wherein M is an integer that is greater than 1. In some embodiments, the capture sequence. In some embodiments, the capture sequence is poly(dT).

In some embodiments, the Round 1 oligonucleotide, the Round 2 oligonucleotide, and/or the Round 3 oligonucleotide each comprises a different capture sequence. In some embodiments, each of all oligonucleotides from M rounds (e.g. Rounds 1, 2, 3, 4, 5 . . . up to M) comprises a different capture sequence, wherein M is an integer that is greater than 1. In some embodiments, each capture sequence is designed to couple to one or more analytes. In some embodiments, each capture sequence is designed to couple to different analytes.

In some embodiments, the analyte is a cell surface protein. In some embodiments, the analyte is a secreted analyte. In some embodiments, the analyte is a secreted protein. In some embodiments, the cell surface protein comprises a proteoglycan and/or a glycoprotein.

In some embodiments, the capture sequence comprised in the Round 1 oligonucleotides is designed to couple to a cell surface protein. In some embodiments, the capture sequence comprised in the Round 1 oligonucleotides is designed to couple to a secreted analyte. In some embodiments, the capture sequence comprised in the Round 2 oligonucleotides is designed to couple to a cell surface protein. In some embodiments, the capture sequence comprised in the Round 2 oligonucleotides is designed to couple to a secreted analyte. In some embodiments, the capture sequence comprised in the Round 3 oligonucleotides is designed to couple to a cell surface protein. In some embodiments, the capture sequence comprised in the Round 3 oligonucleotides is designed to couple to a secreted analyte. In some embodiments, the capture sequence comprised in the Round M oligonucleotides is designed to couple to a cell surface protein. In some embodiments, the capture sequence comprised in the Round M oligonucleotides is designed to couple to a secreted analyte.

In some embodiments, the Round 1 oligonucleotide comprises a capture sequence designed to couple to a first cell surface protein; the Round 2 oligonucleotide comprises a capture sequence designed to couple to a second cell surface protein; and/or the Round 3 oligonucleotide comprises a capture sequence designed to couple to a first secreted analyte.

B. Methods of Generating Master-Copy Arrays

In some aspects, provided herein is a method of patterning a surface in situ for producing a master array on the surface, for example, by one or more rounds of partitioning of beads comprising oligonucleotides into spatially predefined regions (including but not limited to wells), to generate unique DNA sequences in spatial positions in the array, wherein copy arrays can be generated from the master array by migration of the unique DNA sequences.

In some embodiments according to any one of the methods of generating an array described above, the array on the substrate comprising extended nucleic acid molecules, optionally wherein the array on the substrate is a master array. In some embodiments, the array on the substrate comprises the extended nucleic acid molecules, the further extended nucleic acid molecules and/or the even further extended nucleic acid molecules. In some embodiments, the array on the substrate comprises the first extended nucleic acid molecules, the second extended nucleic acid molecules and/or the third extended nucleic acid molecules. In some embodiments, the array on the substrate comprises the first, second, third, fourth, . . . , and/or up to (M)th extended nucleic acid molecules, wherein M is an integer that is greater than 1.

In some embodiments, the extended nucleic acid molecules, the further extended nucleic acid molecules and/or the even further extended nucleic acid molecules are migrated or allowed to migrate into a porous material abutting the well. In some embodiments, the extended nucleic acid molecules, the first extended nucleic acid molecules, the second extended nucleic acid molecules and/or the third extended nucleic acid molecules are migrated or allowed to migrate into a porous material abutting the well. In some embodiments, the one or more of the first, second, third, fourth, . . . , and/or up to (M)th extended nucleic acid molecules are migrated or allowed to migrate into a porous material abutting the well.

In some embodiments according to any one of the methods of providing an array described above, wherein the extended nucleic acid molecules, the further extended nucleic acid molecules, or the even further extended nucleic acid molecules, optionally after cleavage from the substrate and/or amplification (e.g., by PCR), are migrated or allowed to migrate into a porous material abutting the wells.

In some embodiments, provided herein is a method for providing an array, comprising: (a) partitioning a plurality of beads into wells on a substrate, wherein the bead in a first well and the bead in a second well each comprises a different oligonucleotide; (b) disrupting the plurality of beads to release the oligonucleotides, wherein the released oligonucleotides are migrated or allowed to migrate into a porous material abutting the wells, wherein the migration paths in a porous material are substantially parallel to each other; and (c) dividing the porous material into subparts along one or more planes intersecting the migration paths, thereby generating copies of an the array comprising the released oligonucleotides.

In some embodiments, wherein before the migration or the allowance to migrate, the extended nucleic acid molecules, the further extended nucleic acid molecules, and/or the even further extended nucleic acid molecules are subjected to cleavage from the substrate and/or amplification. In some embodiments, wherein before the migration or the allowance to migrate, first extended nucleic acid molecules, the second extended nucleic acid molecules and/or the third extended nucleic acid molecules are subjected to cleavage from the substrate and/or amplification. In some embodiments, wherein before the migration or the allowance to migrate, the first, second, third, fourth, . . . , and/or up to (M)th extended nucleic acid molecules are subjected to cleavage from the substrate and/or amplification.

In some embodiments, the substrate is irradiated (e.g., with UV light) sufficient to cleave nucleic acid molecules off the substrate. In some embodiments, the substrate is irradiated through a mask (e.g., with UV light) sufficient to cleave nucleic acid molecules in the select regions off the substrate. In some embodiments, wherein the nucleic acid molecules are subjected to amplification, the amplification comprises polymerase chain reaction (PCR).

In some embodiments, the porous material is a polymer matrix. In some embodiments, the porous material is a silica matrix. In some embodiments, the porous material is a gel. In some embodiments, the porous material is a hydrogel. In some embodiments, the porous material is a polymer matrix comprising collagen, laminin and/or fibronectin. In some embodiments, the porous material is a polymer matrix comprising hydrogel. In some embodiments, the porous material is a polymer matrix comprising polyacrylamide.

In some embodiments, the migration comprises migration along a field, such as but not limited to migration along an electromagnetic field. In some embodiments, an electromagnetic field is applied across the medium to migrate the nucleic acids and/or oligonucleotides through the medium. In some embodiments, the migration comprises electrophoresis.

In some embodiments, the migration paths in the porous materials are substantially parallel to one another, and the porous material is divided into subparts along one or more planes intersecting the migration paths, thereby generating copies of the array. In some embodiments, any two migration paths in the porous materials vary in direction by no more than about any one of 20°, 10°, 9°, 8°, 7° 6°, 5° 4° 3° 2°, 10, 0.5°, 0.1° or 0.01° angle. In some embodiments, the porous material is divided into subparts along one or more planes that are substantially perpendicular to the mean migration direction. In some embodiments the porous material is divided into subparts along one or more planes that are at about any one of 170°, 1710, 172°, 173°, 174°, 175°, 176°, 177°, 178°, 179°, 180°, 181°, 182°, 183°, 184°, 185°, 186°, 187°, 188°, 189°, or 190° degree to the mean migration direction.

In some embodiments, each of the subparts is divided along one or more planes, wherein a subpart is about any one of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 8 mm, or 10 mm in thickness. In some embodiments, the subpart is about any one of 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1, 1 to 2, 2 to 3, 3 to 4, 4 to 5, 5 to 8, or 8 to 10 mm in thickness. In some embodiments, each of the subparts is divided along one or more planes, wherein each subpart is about any one of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 8 mm, or 10 mm in thickness. In some embodiments, each subpart is about any one of 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1, 1 to 2, 2 to 3, 3 to 4, 4 to 5, 5 to 8, or 8 to 10 mm in thickness.

C. Methods of Generating 3D Spatial Arrays

In some aspects, provided herein is a method of patterning a surface in situ for producing a three-dimensional array on the surface, for example, by one or more rounds of partitioning of beads comprising oligonucleotides into spatially predefined regions (including but not limited to wells), to generate unique DNA sequences in spatial positions in the array, wherein the unique DNA sequences in the array are further migrated.

In some embodiments, provided is a method of generating a three-dimensional array, comprising: (a) partitioning a plurality of beads into wells on a substrate, wherein a first bead in a first well comprises a plurality of first oligonucleotides of varying lengths which comprise the same first barcode sequence, and a second bead in a second well comprises a plurality of second oligonucleotides of varying lengths which comprise the same second barcode sequence; and (b) disrupting the first and second beads to release the oligonucleotides in the corresponding well. In some embodiments, the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the method further comprises cleaving the extended nucleic acid molecules from the substrate, thereby releasing the extended nucleic acid molecules from the substrate. In some embodiments, the released oligonucleotides, or the released extended nucleic acid molecules are migrated or allowed to migrate in a direction into a porous material abutting the wells, wherein the position of a particular released oligonucleotide in the direction correlates with the length of the particular released oligonucleotide, thereby providing in the porous material a 3D-array comprising the released oligonucleotides or released extended nucleic acid molecules.

In some embodiments, provided is a method of generating a three-dimensional array, comprising: (a) partitioning a plurality of beads into wells on a substrate, wherein a first bead in a first well comprises a plurality of first oligonucleotides of varying lengths which comprise the same first barcode sequence, and a second bead in a second well comprises a plurality of second oligonucleotides of varying lengths which comprise the same second barcode sequence; and (b) disrupting the first and second beads to release the oligonucleotides in the corresponding well, wherein the released oligonucleotides are migrated or allowed to migrate in a direction into a porous material abutting the wells, wherein the position of a particular released oligonucleotide in the direction correlates with the length of the particular released oligonucleotide. In some embodiments, the porous material is a three-dimensional medium.

In some embodiments, the first and second wells are comprised within a plurality of N wells, wherein Nis an integer and wherein N has a numerical value greater than any one of 5, 10, 50, 100, 200, 500, 1000, 10000, 20000, 50000, 100000. In some embodiments, the first and second wells are comprised within a plurality of about any one of 1×10¹, 5×10¹, 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², 1×10¹³, 5×10¹³, 1×10¹⁴, 5×10¹⁴, 1×10¹⁵, or 5×10¹⁵ wells.

In some embodiments, the plurality of beads comprises at least the first bead comprising the first plurality of oligonucleotides. In some embodiments, the plurality of beads comprises at least the first bead comprising the plurality of first oligonucleotides. In some embodiments, the plurality of beads comprises at least the third bead comprising the plurality of third oligonucleotides. In some embodiments, the plurality of beads comprises at least the first bead comprising the plurality of first oligonucleotides, the second bead comprising the plurality of second oligonucleotides, and the third bead comprising the plurality of third oligonucleotides. In some embodiments, wherein the first and second wells are comprised within a plurality of N wells, wherein N is an integer, a corresponding plurality of beads comprising up to Nth beads (e.g. first, second, third . . . up to Nth) comprises at least the corresponding N pluralities of oligonucleotides (e.g., a plurality of first oligonucleotides, a plurality of second oligonucleotide etc., up to a plurality of Nth oligonucleotides).

In some embodiments, the oligonucleotides within the plurality of first oligonucleotides each has different regimented lengths. In some embodiments, the oligonucleotides within the plurality of second oligonucleotides each has different regimented lengths. In some embodiments, the oligonucleotides within the plurality of third oligonucleotides each has different regimented lengths. In some embodiments, the oligonucleotides within the plurality of first oligonucleotides and/or the oligonucleotides within the plurality of second oligonucleotides each has different regimented lengths. In some embodiments, the oligonucleotides within the plurality of first oligonucleotides, the oligonucleotides within the plurality of second oligonucleotides, and/or the oligonucleotides within the plurality of third oligonucleotides each has different regimented lengths. In some embodiments, the oligonucleotides in each plurality range in length from any one of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the oligonucleotides in each plurality range in length from 10 to 106 nucleotides. In some embodiments, the oligonucleotides in the plurality have regimented lengths of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the oligonucleotides in the plurality have regimented lengths of between 1 and 100 nucleotides, 100 and 1000 nucleotides or 1000 and 10,000 nucleotides. In some embodiments, the length between a shortest and a longest oligonucleotide in a plurality is about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or 50,000 nucleotides. In some embodiments, the length between a shortest and a longest oligonucleotide in a plurality is any one of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the length between a shortest and a longest oligonucleotide in a plurality is about 100 nucleotides.

As used herein, “regimented” refers to differences in length of oligonucleotides within a population (or plurality) of oligonucleotides. A population (or plurality) of oligonucleotides having regimented lengths means that oligonucleotides in the population (or plurality) differ in length by multiples of a specified number of nucleotides. For example, a population (or plurality) of five oligonucleotides having lengths of 100, 105, 110, 115 and 120 nucleotides could be said to have a regimented length of 5 nucleotides.

In some embodiments according to any of the methods described herein, wherein the porous medium is a three-dimensional medium, the method further comprises migrating the plurality of first oligonucleotides through the three-dimensional medium in a first direction to separate individual oligonucleotides in the first plurality by size and immobilizing the separated oligonucleotides of the first plurality in the medium. In some embodiments, the method further comprises migrating the plurality of second oligonucleotides through the three-dimensional medium in a second direction to separate individual oligonucleotides in the second plurality by size and immobilizing the separated oligonucleotides of the second plurality in the medium. In some embodiments, the method further comprises migrating the plurality of third oligonucleotides through the three-dimensional medium in a third direction to separate individual oligonucleotides in the first plurality by size and immobilizing the separated oligonucleotides of the third plurality in the medium. In some embodiments according to any of the methods described herein, wherein the porous medium is a three-dimensional medium, the method further comprises: (a) migrating the plurality of first oligonucleotides through the three-dimensional medium in a first direction to separate individual oligonucleotides in the first plurality by size and immobilizing the separated oligonucleotides of the first plurality in the medium; and/or (b) migrating the plurality of second oligonucleotides through the three-dimensional medium in a second direction to separate individual oligonucleotides in the second plurality by size and immobilizing the separated oligonucleotides of the second plurality in the medium; and/or (c) migrating the plurality of third oligonucleotides through the three-dimensional medium in a third direction to separate individual oligonucleotides in the first plurality by size and immobilizing the separated oligonucleotides of the third plurality in the medium.

In some embodiments, the method further comprises analyzing the three-dimensional medium to determine the relative locations of the oligonucleotides of the three pluralities. In some embodiments, the oligonucleotides in the pluralities are separated based on size or length of migration through the medium. In some embodiments, the length of migration through the medium is proportional to the size of the oligonucleotide (such as the length and/or regimented length of the oligonucleotide).

In some embodiments, wherein the porous material is a three-dimensional medium, the porous material is a polymer matrix. In some embodiments, the porous material is a silica matrix. In some embodiments, the porous material is a gel. In some embodiments, the porous material is a hydrogel. In some embodiments, the porous material is a polymer matrix comprising collagen, laminin and/or fibronectin. In some embodiments, the porous material is a polymer matrix comprising hydrogel. In some embodiments, the porous material is a polymer matrix comprising polyacrylamide.

In some embodiments, the migration comprises migration along a field, such as but not limited to migration along an electromagnetic field. In some embodiments, an electromagnetic field is applied across the medium to migrate the oligonucleotides through the medium. In some embodiments, the migration comprises electrophoresis.

In some embodiments, wherein prior to the migrating and the immobilizing of the pluralities of oligonucleotides, the method comprises embedding a cell sample and/or a tissue sample in the three-dimensional medium (such as a polymer matrix). In some embodiments, wherein prior to the migrating and the immobilizing of the pluralities of oligonucleotides, the method comprises embedding a cell sample and/or a tissue sample in the three-dimensional medium (such as a polymer matrix). In some embodiments, the medium contains cellular RNAs. In some embodiments, wherein prior to the migrating and the immobilizing of the pluralities of oligonucleotides, the method comprises capturing cellular RNAs in the medium using RNA capturing probes and/or production of RNA-derived amplification products.

In some embodiments, the oligonucleotides of the first, second and third pluralities comprise a hybridizing nucleotide sequence capable of identifying the same cellular RNA. In some embodiments, the oligonucleotides of all pluralities comprise a hybridizing nucleotide sequence capable of identifying the same cellular RNA.

In some embodiments, the oligonucleotides of the first, second and third pluralities comprise hybridizing nucleotide sequences capable of identifying different cellular RNAs. In some embodiments, the oligonucleotides of all pluralities comprise hybridizing nucleotide sequences capable of identifying different cellular RNAs.

In some embodiments according to any one of the methods described above, the method further comprises subjecting the medium containing the separated oligonucleotides to conditions where the hybridizing nucleotide sequence of the separated oligonucleotides hybridizes to the captured cellular RNAs, RNA capturing probes and/or RNA-derived amplification products present in the medium that have a nucleotide sequence complementary to the hybridizing nucleotide sequence of the oligonucleotides.

In some embodiments, provided is a method comprising: (a) capturing cellular RNAs in a three-dimensional tissue sample using RNA capturing probes and/or sequence-specific amplification of the cellular RNAs; (b) embedding the tissue sample in a conductive polymer; (c) partitioning a plurality of beads into wells on a substrate, wherein a first bead in a first well comprises a plurality of first oligonucleotides of varying lengths which comprise the same first barcode sequence, a second bead in a second well comprises a plurality of second oligonucleotides of varying lengths which comprise the same second barcode sequence and a third bead in a third well comprises a plurality of third oligonucleotides of varying lengths which comprise the same third barcode sequence; further wherein the first, second and third pluralities of oligonucleotides encode i) hybridizing nucleotide sequences capable of identifying different RNAs, or ii) nucleotide sequences capable of hybridizing to RNA, RNA capturing probes, or the RNA-derived amplification products that are different; (d) placing the conductive polymer abutting the wells on the substrate; (e) disrupting the first, second and third beads to release the oligonucleotides in the corresponding wells, wherein the released oligonucleotides are allowed to migrate into the conductive polymer abutting the wells, wherein a position of a particular released oligonucleotide in the direction correlates with the length of the particular released oligonucleotide; (f) electrophoresing the first plurality of oligonucleotides through a first dimension of the conductive polymer to separate individual oligonucleotides of the first composition by size, and immobilizing the separated oligonucleotides of the first plurality in the polymer; electrophoresing the second plurality of oligonucleotides through a second dimension of the conductive polymer to separate individual oligonucleotides of the second composition by size, and immobilizing the separated oligonucleotides of the second plurality in the polymer; electrophoresing the second plurality of oligonucleotides through a third dimension of the conductive polymer to separate individual oligonucleotides of the third composition by size, and immobilizing the separated oligonucleotides of the third pluralities in the polymer; (g) subjecting the conductive polymer to conditions providing for hybridization of oligonucleotides in the polymer to the captured RNAs, RNA capturing probes and/or RNA-derived amplification products; (h) performing nucleotide sequencing of the hybridized oligonucleotides and captured RNAs, RNA capturing probes and/or RNA-derived amplification products; and (i) integrating sizes of the sequenced oligonucleotides with identity of the captured cellular RNAs to determine relative location of the cellular RNAs in the three-dimensions of the polymer.

In some embodiments, the oligonucleotides are gradient-tagging, wherein migration of the oligonucleotides creates a concentration gradient along the migration path. In instances where the barcoded probes on an array are generated through ligation of two or more oligonucleotides, a concentration gradient of the oligonucleotides can be applied to a substrate such that different combinations of the oligonucleotides are incorporated into a barcoded probe depending on its location on the substrate.

In some embodiments, the directions or dimensions of migration for any of two pluralities of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the directions or dimensions of migration for any of two pluralities of oligonucleotides intersect orthogonally or perpendicularly at an angle of 90 degrees, or about orthogonally or perpendicularly at an angle of about 90 degrees.

In some embodiments, the direction or dimension of migration for the first plurality of oligonucleotides and the direction or dimension of migration for the second plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the first plurality of oligonucleotides and the direction or dimension of migration for the second plurality of oligonucleotides intersect orthogonally or perpendicularly at an angle of 90 degrees, or about orthogonally or perpendicularly at an angle of about 90 degrees.

In some embodiments, the direction or dimension of migration for the first plurality of oligonucleotides and the direction or dimension of migration for the third plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the first plurality of oligonucleotides and the direction or dimension of migration for the third plurality of oligonucleotides intersect orthogonally or perpendicularly at an angle of 90 degrees, or about orthogonally or perpendicularly at an angle of about 90 degrees.

In some embodiments, the direction or dimension of migration for the second plurality of oligonucleotides and the direction or dimension of migration for the third plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the second plurality of oligonucleotides and the direction or dimension of migration for the third plurality of oligonucleotides intersect orthogonally or perpendicularly at an angle of 90 degrees, or about orthogonally or perpendicularly at an angle of about 90 degrees.

In some embodiments, the directions or dimensions of migration for the first, second and third pluralities of oligonucleotides intersect one another at an angle of about 85, about 80, about 75, about 70, about 65, about 60, about 55, about 50, about 45, about 40, about 35, about 30, about 25, about 20, about 15, about 10, or about 5 degrees. In some embodiments, the directions or dimensions of migration for the first, second and third pluralities of oligonucleotides intersect one another at an angle of 90 degrees, or about orthogonally or perpendicularly at an angle of about 90 degrees.

Disclosed here are oligonucleotides for use in the methods described in this generation of 3-D array. In some examples, the disclosed oligonucleotides have different lengths. In some examples, the disclosed oligonucleotides encode a nucleotide sequence that correlates with or is linked to length or size of the oligonucleotide. These sequences, when determined in an oligonucleotide by nucleotide sequencing, can be used to identify the length of the oligonucleotide. These nucleotide sequences may be barcode nucleotide sequences, as known in the art. The nucleotide sequences that are linked to or correlate with oligonucleotide length may be called length barcode sequences.

In some examples, the oligonucleotides disclosed here encode a nucleotide sequence that is capable of hybridizing to or identifying another nucleic acid molecule. These sequences within the disclosed oligonucleotides may be referred to as hybridizing nucleotide sequences and may be used to identify specific target nucleic acid molecules by specifically hybridizing to complementary sequences within the molecules. In some examples, the specific target nucleic acid molecules identified are RNA molecules. These hybridizing nucleotide sequences in an oligonucleotide, in various examples, may be designed to hybridize to a specific RNA molecule, to a probe that has specifically hybridized to the RNA molecule, and/or to a nucleic acid molecule that has been synthesized, amplified or ligated using the specific RNA molecule as a template.

In other embodiments, the hybridizing nucleotide sequences may be designed to hybridize to a reporter oligonucleotide that corresponds to a labelling agent. The labeling agent may be a protein binding agent that comprises a reporter oligonucleotide (e.g., an antibody conjugated to a reporter oligonucleotide which identifies the antibody) or a cell labeling agent that comprises a reporter oligonucleotide (e.g., either an antibody that specifically binds an extracellular protein or a lipid-based molecule conjugated to a reporter oligonucleotide). The cell labelling agent may be added to the tissue sample prior to the migration of oligonucleotides as described herein (e.g., one or more different cell labelling agent(s) having different reporter oligonucleotides may be added to specific region(s) of a tissue sample) and the reporter oligonucleotides may be detected downstream. Other cell labelling agents include, without limitation, a lipophilic moiety (e.g., cholesterol), a nanoparticle, a cell-penetrating peptide, a peptide-based chemical vector, a dye, and a fluorophore. Those of ordinary skill in the art will appreciate that other labeling agents with reporter oligonucleotides that are suitable for use in the present disclosure (see US Published Patent Application Nos. US20200002763 A1, US20190367969 A1, US 20190323088 A1, the contents of each of which are incorporated herein by reference in their entireties). In some examples, specific hybridizing nucleotide sequences may correlate with or be linked to a nucleotide sequence within the oligonucleotide. These sequences, when ascertained in an oligonucleotide by nucleotide sequencing, can be used to identify the specific hybridizing nucleotide sequence within an oligonucleotide (or a derivative thereof), and/or to identify a cellular analyte (e.g., an RNA) to which the hybridizing nucleotide sequence has hybridized, a probe that has hybridized to the cellular analyte (e.g., a specific RNA), and/or a nucleic acid molecule that has been synthesized, amplified or ligated using the cellular analyte (e.g., a specific RNA molecule) as a template, and/or a reporter oligonucleotide that identifies a labeling agent (as described herein). These nucleotide sequences may be barcode nucleotide sequences, as known in the art.

In some examples, oligonucleotides the disclosed oligonucleotides may encode a barcode nucleotide sequence linked to length of the oligonucleotides, a hybridizing nucleotide sequence and barcode nucleotide sequence linked to the specific hybridizing nucleotide sequence.

Also disclosed here are populations of oligonucleotides as described above. Some example populations of oligonucleotides contain oligonucleotides of different lengths.

In some examples, oligonucleotides in a population of oligonucleotides, or in a combination of populations, may encode an additional barcode sequence. This barcode sequence may be used to determine in which dimension the oligonucleotides in the population, or in a combination of populations, were migrated. For example, oligonucleotides migrated through a multidimensional space in a x-dimension may encode a common first barcode nucleotide sequence. Oligonucleotides migrated through the multidimensional space in a y-dimension may encode a common second barcode nucleotide sequence. Oligonucleotides migrated through the multidimensional space in a z-dimension may encode a common third barcode nucleotide sequence. After the migration, identification of these barcodes in an oligonucleotide provides information on the dimension through which the oligonucleotide was migrated.

D. Wells, Characteristics Thereof, and Methods of Forming Wells on Substrate

In some embodiments, the method comprises partitioning beads into at least the first well and the second well. In some embodiments, the first and/or second wells are formed by a material enclosing a first region and a second region, respectively, on the substrate. In some embodiments, the first and/or second wells are formed by etching a layer of the material on the substrate on the substrate. In some embodiments, the one or more wells are formed by etching a layer of the material on the substrate.

In some embodiments, the first and second wells are comprised within a plurality of N wells, wherein Nis an integer and wherein N has a numerical value greater than any one of 5, 10, 50, 100, 200, 500, 1000, 10000, 20000, 50000, 100000. In some embodiments, the first and second wells are comprised within a plurality of about any one of 1×10¹, 5×10¹, 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², 1×10¹³, 5×10¹³, 1×10¹⁴, 5×10¹⁴, 1×10¹⁵, or 5×10¹⁵ wells.

In some embodiments according to any one of the methods described herein, wherein prior to partitioning the beads, the substrate is coated with a photoresist layer. In some embodiments, the substrate is coated with a photoresist layer using spin coating or dipping. In some embodiments, the one or more wells are formed by etching a layer of a photoresist on the substrate. In some embodiments, the one or more wells are formed by etching a layer of a photoresist at the corresponding regions on the substrate. In some embodiments, the first and/or second wells are formed by etching a layer of photoresist on the substrate. In some embodiments, the first and/or second wells are formed by etching a layer of a photoresist at the corresponding first and/or second regions on the substrate.

In some embodiments, the method comprises partitioning beads into one or more wells. In some embodiments, the one or more wells are formed by a material enclosing one or more corresponding regions on the substrate. In some embodiments, the one or more wells are formed by etching a layer of the material on the substrate. In some embodiments, the nucleic acid molecules are immobilized on the substrate prior to the etching step. In some embodiments, the nucleic acids are immobilized in one or more regions prior to the etching step. In some embodiments, the nucleic acids are immobilized in the first and second regions prior to the etching step. In some embodiments, the nucleic acids are provided in one or more wells after the etching step. In some embodiments, the nucleic acids are provided in the first and second wells after the etching step. In some embodiments, the nucleic acids are immobilized in one or more regions after the etching step. In some embodiments, the nucleic acids are immobilized in the first and second regions after the etching step.

In some embodiments, the first region and the second region are comprised within a plurality of regions, wherein the plurality of regions are arranged in a square array.

In some embodiments, the first and second regions are of about 1 μm to about 40 μm in average diameter. In some embodiments, the first and second regions are of about 2 μm to about 20 μm in average diameter. In some embodiments, the first and second regions are of about any one of: 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm or 40 μm in average diameter. In some embodiments, the first and second regions are of about 5 μm in diameter.

In some embodiments, the opening and/or boundary between regions is between about 0.5 μm and about 40 μm in length and between about 0.05 μm and about 4 μm in width. In some embodiments, the opening and/or the boundary is between about 1 μm and about 20 μm in length and between about 0.1 μm and about 2 μm in width. In some embodiments, the opening and/or the boundary is about any one of: 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 25 μm, 30 μm or 40 μm in length. In some embodiments, the opening and/or the boundary region is about any one of: 0.05 μm, 0.1 μm, 0.2 μm, 0.3 μm, 0.4 μm, 0.5 μm, 0.6 μm, 0.7 μm, 0.8 μm, 0.9 μm, 1.0 μm, 1.1 μm, 1.2 μm, 1.3 μm, 1.4 μm, 1.5 μm, 1.6 μm, 1.7 μm, 1.8 μm, 1.9 μm, 2.0 μm, 2.5 μm, 3.0 μm or 4.0 μm in width.

E. Photoresist

A photoresist is a light-sensitive material used in processes (such as photolithography and photoengraving) to form a pattern on a surface. A photoresist may comprise a polymer, a sensitizer, and/or a solvent. The photoresist composition used herein is not limited to any specific proportions of the various components.

Photoresists can be classified as positive or negative. In positive photoresists, the photochemical reaction that occurs during light exposure weakens the polymer, making it more soluble to developer, so a positive pattern is achieved. In the case of negative photoresists, exposure to light causes polymerization of the photoresist, and therefore the negative photoresist remains on the surface of the substrate where it is exposed, and the developer solution removes only the unexposed areas. In some embodiments, the photoresist used herein is a positive photoresist. In some embodiments, the photoresist is removable with UV light.

The photoresist may experience changes in pH upon irradiation. In some embodiments, the photoresist on the substrate comprises a photoacid generator (PAG). In some embodiments, the photoacid generator or photoacid generators irreversibly release protons upon absorption of light. Photoacid generators may be used as components of photocurable polymer formulations and chemically amplified photoresists. Examples of photoacid generators include triphenylsulfonium triflate, diphenylsulfonium triflate, diphenyliodonium nitrate, N-Hydroxynaphthalimide triflate, triarylsulfonium hexafluorophosphate salts, N-hydroxy-5-norbornene-2,3-dicarboximide perfluoro-1-butanesulfonate, bis(4-tert-butylphenyl)iodonium perfluoro-1-butanesulfonate, etc.

In some embodiments, the photoresist further comprises an acid scavenger. In some embodiments, an acid scavenger acts to neutralize, adsorb and/or buffer acids, and may comprise a base or alkaline compound. In some embodiments, acid scavengers act to reduce the amount or concentration of protons or protonated water. In some embodiments, an acid scavenger acts to neutralize, diminish, or buffer acid produced by a photoacid generator. In some embodiments, an acid scavenger exhibits little or no stratification over time or following exposure to heat. In some embodiments, acid scavengers may be further subdivided into “organic bases” and “polymeric bases.” A polymeric base is an acid scavenger (e.g., basic unit) attached to a longer polymeric unit. A polymer is typically composed of a number of coupled or linked monomers. The monomers can be the same (to form a homopolymer) or different (to form a copolymer). In a polymeric base, at least some of the monomers act as acid scavengers. An organic base is a base which is joined to or part of a non-polymeric unit. Non-limiting examples of organic bases include, without limitation, amine compounds (e.g., primary, secondary and tertiary amines). Generally any type of acid scavenger, defined here as a traditional Lewis Base, an electron pair donor, can be used in accordance with the present disclosure.

In some embodiments, the photoresist further comprises a base quencher. Base quenchers may be used in photoresist formulations to improve performance by quenching reactions of photoacids that diffuse into unexposed regions. Base quenchers may comprise aliphatic amines, aromatic amines, carboxylates, hydroxides, or combinations thereof. Examples of base quenchers include but are not limited to, trioctylamine, 1,8-diazabicyclo[5.4.0]undec-7-ene (DBU), 1-piperidineethanol (1PE), tetrabutylammonium hydroxide (TBAH), dimethylamino pyridine, 7-diethylamino-4-methyl coumarin (Coumarin 1), tertiary amines, sterically hindered diamine and guanidine bases such as 1,8-bis(dimethylamino)naphthalene (PROTON SPONGE), berberine, or polymeric amines such as in the PLURONIC or TETRONIC series commercially available from BASF.

In some embodiments, the photoresist further comprises a photosensitizer. A photosensitizer is a molecule that produces a chemical change in another molecule in a photochemical process. Photosensitizers are commonly used in polymer chemistry in reactions such as photopolymerization, photocrosslinking, and photodegradation. Photosensitizers generally act by absorbing ultraviolet or visible region of electromagnetic radiation and transferring it to adjacent molecules. In some embodiments, photosensitizer shifts the photo sensitivity to a longer wavelength of electromagnetic radiation. The sensitizer, also called a photosensitizer, is capable of activating the PAG at, for example, a longer wavelength of light in accordance with an aspect of the present disclosure. Preferably, the concentration of the sensitizer is greater than that of the PAG, such as 1.1 times to 5 times greater, for example, 1.1 times to 3 times greater the concentration of PAG. Exemplary sensitizers suitable for use in the disclosure include but are not limited to, isopropylthioxanthone (ITX) and 10H-phenoxazine (PhX).

In some embodiments, the photoresist further comprises a matrix. The matrix generally refers to polymeric materials that may provide sufficient adhesion to the substrate when the photoresist formulation is applied to the top surface of the substrate, and may form a substantially uniform film when dissolved in a solvent and spread on top of a substrate. Examples of a matrix may include, but are not limited to, polyester, polyimide, polyethylene naphthalate (PEN), polyvinyl chloride (PVC), polymethylmethacrylate (PMMA), polyglycidalmethacrylate (PGMA), and polycarbonate, or a combination thereof. The matrix may be chosen based on the wavelength of the radiation used for the generation of acid when using the photoresist formulation, the adhesion properties of the matrix to the top surface of the substrate, the compatibility of the matrix to other components of the formulation, and the ease of removable or degradation (if needed) after use. In some embodiments, the photoresist in the first and the second region comprises the same matrix. In some embodiments, the photoresist in the first and the second region comprises different matrices.

In some embodiments, the photoresist further comprises a surfactant. Surfactants may be used to improve coating uniformity, and may include ionic, non-ionic, monomeric, oligomeric, and polymeric species, or combinations thereof. Examples of possible surfactants include fluorine-containing surfactants such as the FLUORAD series available from 3M Company in St. Paul, Minn., and siloxane-containing surfactants such as the SILWET series available from Union Carbide Corporation in Danbury, Conn.

In some embodiments, the photoresist further comprises a casting solvent. A casting solvent may be used so that the photoresist may be applied evenly on the substrate surface to provide a defect-free coating. Examples of suitable casting solvents may include ethers, glycol ethers, aromatic hydrocarbons, ketones, esters, ethyl lactate, γ-butyrolactone, cyclohexanone, ethoxyethylpropionate (EEP), a combination of EEP and gamma-butyrolactone (GBL), and propylene glycol methyl ether acetate (PGMEA).

Methods of applying photoresist to the substrate include, but are not limited to, dipping, spreading, spraying, or any combination thereof. In some embodiments, the photoresist is applied via spin coating, thereby forming a photoresist layer on the substrate.

In some embodiments, the photoresist is in direct contact with the oligonucleotides on the substrate. In some embodiments, the oligonucleotide molecules on the substrate are embedded in the photoresist. In some embodiments, the photoresist is not in direct contact with the oligonucleotides. In some embodiments, oligonucleotide molecules on the substrate are embedded in an underlayer that is underneath the photoresist. For example, oligonucleotide molecules on the substrate may be embedded in a soluble polymer underlayer (e.g., a soluble polyimide underlayer (XU-218)), and the photoresist forms a photoresist layer on top of the underlayer.

In some embodiments, the photoresist may be removed and re-applied one or more times. For example, the photoresist may be stripped from the substrate and/or the oligonucleotides ligated to the substrate. Removal of photoresist can be accomplished with various degrees of effectiveness. In some embodiments, the photoresist is completely removed from the substrate and/or the oligonucleotides ligated to the substrate before re-application. Methods of removing photoresist may include, but are not limited to, using organic solvent mixtures, using liquid chemicals, exposure to a plasma environment, or other dry techniques such as UV/O₃ exposure. In some embodiments, the photoresist is stripped using organic solvent.

F. Molecular Arrays

In some aspects, the method provided herein comprises attaching oligonucleotides (e.g. a barcode) to a substrate. Oligonucleotides may be attached to the substrate according to the methods set forth in U.S. Pat. Nos. 6,737,236, 7,259,258, 7,375,234, 7,427,678, 5,610,287, 5,807,522, 5,837,860, and 5,472,881; U.S. Patent Application Publication Nos. 2008/0280773 and 2011/0059865; Shalon et al. (1996) Genome Research, 639-645; Rogers et al. (1999) Analytical Biochemistry 266, 23-30; Stimpson et al. (1995) Proc. Natl. Acad. Sci. USA 92, 6379-6383; Beattie et al. (1995) Clin. Chem. 45, 700-706; Lamture et al. (1994) Nucleic Acids Research 22, 2121-2125; Beier et al. (1999) Nucleic Acids Research 27, 1970-1977; Joos et al. (1997) Analytical Biochemistry 247, 96-101; Nikiforov et al. (1995) Analytical Biochemistry 227, 201-209; Timofeev et al. (1996) Nucleic Acids Research 24, 3142-3148; Chrisey et al. (1996) Nucleic Acids Research 24, 3031-3039; Guo et al. (1994) Nucleic Acids Research 22, 5456-5465; Running and Urdea (1990) BioTechniques 8, 276-279; Fahy et al. (1993) Nucleic Acids Research 21, 1819-1826; Zhang et al. (1991) 19, 3929-3933; and Rogers et al. (1997) Gene Therapy 4, 1387-1392. The entire contents of each of the foregoing documents are incorporated herein by reference.

Arrays can be prepared by a variety of methods. In some embodiments, arrays are prepared through the synthesis (e.g., in situ synthesis) of oligonucleotides on the array, or by jet printing or lithography. For example, light-directed synthesis of high-density DNA oligonucleotides can be achieved by photolithography or solid-phase DNA synthesis. To implement photolithographic synthesis, synthetic linkers modified with photochemical protecting groups can be attached to a substrate and the photochemical protecting groups can be modified using a photolithographic mask (applied to specific areas of the substrate) and light, thereby producing an array having localized photo-deprotection. Many of these methods are known in the art, and are described e.g., in Miller et al., “Basic concepts of microarrays and potential applications in clinical microbiology.” Clinical microbiology reviews 22.4 (2009): 611-633; US201314111482A; U.S. Pat. No. 9,593,365B2; US2019203275; and WO2018091676, the contents of all of which are incorporated herein by reference in their entireties. In some embodiments, arrays are prepared through synthesis (e.g., in situ synthesis) of oligonucleotides on the array by partitioning of beads comprising oligonucleotides and/or splints into pre-formed wells.

In some embodiments, a substrate comprising an array of molecules is provided, e.g., in the form of a lawn of polymers (e.g., oligonucleotides) or polymers on the substrate in a pattern. Examples of polymers on an array may include, but are not limited to, nucleic acids, peptides, phospholipids, polysaccharides, heteromacromolecules in which a known drug is covalently bound to any of the above, polyurethanes, polyesters, polycarbonates, polyureas, polyamides, polyethyleneimines, polyarylene sulfides, polysiloxanes, polyimides, and polyacetates. The molecules occupying different features of an array typically differ from one another, although some redundancy in which the same polymer occupies multiple features can be useful as a control. For example, in a nucleic acid array, the nucleic acid molecules within the same feature are typically the same, whereas nucleic acid molecules occupying different features are mostly different from one another.

In some examples, the molecules on the array may be nucleic acids. The nucleic acid molecule can be single-stranded or double-stranded. Nucleic acid molecules on an array may be DNA or RNA. The DNA may be single-stranded or double-stranded. The DNA may include, but are not limited to, mitochondrial DNA, cell-free DNA, complementary DNA (cDNA), genomic DNA, plasmid DNA, cosmid DNA, bacterial artificial chromosome (BAC), or yeast artificial chromosome (YAC). The RNA may include, but are not limited to, mRNAs, tRNAs, snRNAs, rRNAs, retroviruses, small non-coding RNAs, microRNAs, polysomal RNAs, pre-mRNAs, intronic RNA, viral RNA, cell free RNA and fragments thereof. The non-coding RNA, or ncRNA can include snoRNAs, microRNAs, siRNAs, piRNAs and long nc RNAs.

In some embodiments, the molecules on an array comprise oligonucleotide barcodes. A barcode sequence can be of varied length. In some embodiments, the barcode sequence is about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 65, or about 70 nucleotides in length. In some embodiments, the barcode sequence is between about 4 and about 25 nucleotides in length. In some embodiments, the barcode sequences is between about 10 and about 50 nucleotides in length. The nucleotides can be completely contiguous, e.g., in a single stretch of adjacent nucleotides, or they can be separated into two or more separate subsequences that are separated by 1 or more nucleotides. In some embodiments, the barcode sequence can be about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at least about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or longer. In some embodiments, the barcode sequence can be at most about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides or shorter.

The oligonucleotide can include one or more (e.g., two or more, three or more, four or more, five or more) Unique Molecular Identifiers (UMIs). A unique molecular identifier is a contiguous nucleic acid segment or two or more non-contiguous nucleic acid segments that function as a label or identifier for a particular analyte, or for a capture probe that binds a particular analyte (e.g., via the capture domain).

A UMI can be unique. A UMI can include one or more specific polynucleotides sequences, one or more random nucleic acid and/or amino acid sequences, and/or one or more synthetic nucleic acid and/or amino acid sequences.

In some embodiments, the UMI is a nucleic acid sequence that does not substantially hybridize to analyte nucleic acid molecules in a biological sample. In some embodiments, the UMI has less than 90% sequence identity (e.g., less than 80%, 70%, 60%, 50%, or less than 40% sequence identity) to the nucleic acid sequences across a substantial part (e.g., 80% or more) of the nucleic acid molecules in the biological sample.

The UMI can include from about 6 to about 20 or more nucleotides within the sequence of capture probes, e.g., barcoded oligonucleotides in an array generated using a method disclosed herein. In some embodiments, the length of a UMI sequence can be about 6, 7, 8, 9, 10, 11, 12, 13, 14, 15, 16, 17, 18, 19, 20 nucleotides or longer. In some embodiments, the length of a UMI sequence can be at least about 6, 7, 8, 9, 10, 11, 12, 13, 14, 15, 16, 17, 18, 19, 20 nucleotides or longer. In some embodiments, the length of a UMI sequence is at most about 6, 7, 8, 9, 10, 11, 12, 13, 14, 15, 16, 17, 18, 19, 20 nucleotides or shorter. These nucleotides can be contiguous, e.g., in a single stretch of adjacent nucleotides, or they can be separated into two or more separate subsequences that are separated by 1 or more nucleotides. Separated UMI subsequences can be from about 4 to about 16 nucleotides in length. In some embodiments, the UMI subsequence can be about 4, 5, 6, 7, 8, 9, 10, 11, 12, 13, 14, 15, 16 nucleotides or longer. In some embodiments, the UMI subsequence can be at least about 4, 5, 6, 7, 8, 9, 10, 11, 12, 13, 14, 15, 16 nucleotides or longer. In some embodiments, the UMI subsequence can be at most about 4, 5, 6, 7, 8, 9, 10, 11, 12, 13, 14, 15, 16 nucleotides or shorter.

In some embodiments, a UMI is attached to other parts of the nucleotide in a reversible or irreversible manner. In some embodiments, a UMI is added to, for example, a fragment of a DNA or RNA sample before, during, and/or after sequencing of the analyte. In some embodiments, a UMI allows for identification and/or quantification of individual sequencing-reads. In some embodiments, a UMI is a used as a fluorescent barcode for which fluorescently labeled oligonucleotide probes hybridize to the UMI.

In some embodiments, a method provided herein further comprises a step of providing the substrate. A wide variety of different substrates can be used for the foregoing purposes. In general, a substrate can be any suitable support material. The substrate may comprise materials of one or more of the IUPAC Groups 4, 6, 11, 12, 13, 14, and 15 elements, plastic material, silicon dioxide, glass, fused silica, mica, ceramic, or metals deposited on the aforementioned substrates. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate.

A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments, the surface of the substrate is coated. In some embodiments, the surface of the substrate is coated with a photoresist. In some embodiments, the method described herein comprises applying the photoresist to the substrate. In some embodiments, the substrate comprises a pattern of oligonucleotide molecules on the substrate prior to photoresist(s) being applied to the substrate. In some embodiments, the substrate does not comprise a pattern of oligonucleotide molecules on the substrate prior to photoresist(s) being applied to the substrate. In some embodiments where the substrate does not comprise oligonucleotide molecules prior to the application of photoresist(s), the substrate comprises a plurality of functional groups. In some embodiments, the plurality of functional groups of the substrate are not protected, for example, by photo-sensitive groups, moieties, or molecules. In some embodiments, the plurality of functional groups are aldehyde groups. In some embodiments, the plurality of functional groups of the substrate are click chemistry groups. The click chemistry group may be capable of various chemical reactions, which include but are not limited to, a nucleophilic addition reaction, a cyclopropane-tetrazine reaction, a strain-promoted azide-alkyne cycloaddition (SPAAC) reaction, an alkyne hydrothiolation reaction, an alkene hydrothiolation reaction, a strain-promoted alkyne-nitrone cycloaddition (SPANC) reaction, an inverse electron-demand Diels-Alder (IED-DA) reaction, a cyanobenzothiazole condensation reaction, an aldehyde/ketone condensation reaction, or a Cu(I)-catalyzed azide-alkyne cycloaddition (CuAAC) reaction. In some embodiments, the plurality of functional groups on the substrate react with functional groups in functionalized oligonucleotide molecules. In some embodiments, the functional groups in the functionalized oligonucleotide molecules are amino groups. In some embodiments, the functionalized oligonucleotide molecules are 5′ amine-terminated.

In some embodiments, during the contact between the functionalized oligonucleotide molecules and the functional groups on the substrate, the substrate is heated to dryness. In some embodiments, after the contact between the functionalized oligonucleotide molecules and the functional groups on the substrate, the substrate is heated to dryness. In some embodiments according to any one of the methods described herein, the method further comprises blocking unreacted functional groups of the substrate. In some embodiments, the method comprises rendering the reaction between functional groups of the substrate and the functionalized oligonucleotide molecules irreversible. For example, the aldehyde groups of the substrate are reacted with 5′ amino groups of the functionalized oligonucleotide molecules, and the substrate is contacted with a reagent to block unreacted aldehyde groups and render the reaction irreversible. In some embodiment, the reagent is a reductive agent. In some embodiment, the reagent is sodium borohydride.

### III. Hybridization/Ligation

The nucleotide barcode parts described herein may be linked via phosphodiester bonds. The nucleotide barcode parts may also be linked via non-natural oligonucleotide linkages such as methylphosphonate or phosphorothioate bonds, via non-natural biocompatible linkages such as click-chemistry, via enzymatic biosynthesis of nucleic acid polymers such as by polymerase or transcriptase, or a combination thereof. Ligation may be achieved using methods that include, but are not limited to, primer extension, hybridization ligation, and chemical ligation. In some embodiments, the oligonucleotide comprising the barcode sequence is hybridized to a splint which is in turn hybridized to an oligonucleotide molecule in the unmasked region. The oligonucleotide comprising the barcode sequence may be further ligated to the oligonucleotide in the unmasked region to generate a barcoded oligonucleotide molecule. In some embodiments, the oligonucleotide comprising the barcode sequence is hybridized to a splint which is in turn hybridized to an oligonucleotide molecule in a well (or a region corresponding to a well). The oligonucleotide comprising the barcode sequence may be further ligated to the nucleic acids in a well (or in a region corresponding to a well) to generate a barcoded oligonucleotide molecule.

In some cases, a primer extension or other amplification reaction may be used to synthesize an oligonucleotide on a substrate via a primer attached to the substrate. In such cases, a primer attached to the substrate may hybridize to a primer binding site of an oligonucleotide that also contains a template nucleotide sequence. The primer can then be extended by a primer extension reaction or other amplification reaction, and an oligonucleotide complementary to the template oligonucleotide can thereby be attached to the substrate.

In some embodiments, chemical ligation can be used to ligate two or more oligonucleotides. In some embodiments, chemical ligation involves the use of condensing reagents. In some embodiments, condensing reagents are utilized to activate a phosphate group. In some embodiments, condensing reagents may be one or more of 1-ethyl-3-(3-dimethylaminopropyl) carbodiimide (EDCI), cyanogen bromide, imidazole derivatives, and 1-hydroxybenzotriazole (HOAt). In some embodiments, functional group pairs selected from one or more of a nucleophilic group and an electrophilic group, or an alkyne and an azide group are used for chemical ligation. In some embodiments, chemical ligation of two or more oligonucleotides requires a template strand that is complementary to the oligonucleotides to be ligated (e.g., a splint). In some embodiments, the chemical ligation process is similar to oligonucleotide synthesis.

A splint is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complementary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together.

Splints have been described, for example, in US20150005200A1, the content of which is herein incorporated by reference in its entirety. A splint may be used for ligating two oligonucleotides. The sequence of a splint may be configured to be in part complementary to at least a portion of the first oligonucleotides that are attached to the substrate and in part complementary to at least a portion of the second oligonucleotides. In one case, the splint can hybridize to the second oligo via its complementary sequence; once hybridized, the second oligonucleotide or oligonucleotide segment of the splint can then be attached to the first oligonucleotide attached to the substrate via any suitable attachment mechanism, such as, for example, a ligation reaction. The splint complementary to both the first and second oligonucleotides can then be then denatured (or removed) with further processing. The method of attaching the second oligonucleotides to the first oligonucleotides can then be optionally repeated to ligate a third, and/or a fourth, and/or more parts of the barcode onto the array with the aid of splint(s). In some embodiments, the splint is between 10 and 50 oligonucleotides in length, e.g., between 10 and 45, 10 and 40, 10 and 35, 10 and 30, 10 and 25, or 10 and 20 oligonucleotides in length. In some embodiments, the splint is between 15 and 50, 15 and 45, 15 and 40, 15 and 35, 15 and 30, 15 and 30, or 15 and 25 nucleotides in length.

In some embodiments, the released Round 1 oligonucleotide comprises a sequence that hybridizes to a Round 1 splint which in turn hybridizes to the nucleic acid molecules in a particular well. In some embodiments, the released Round 1 oligonucleotide is ligated to the nucleic acid molecules using the Round 1 splint as template to generate the (first) extended nucleic acid molecules. In some embodiments, the released Round 2 oligonucleotide comprises a sequence that hybridizes to a Round 2 splint which in turn hybridizes to the (first) extended nucleic acid molecules in a particular well. In some embodiments, the released Round 2 oligonucleotide is ligated to the (first) extended nucleic acid molecules using the Round 2 splint as template to generate the further extended nucleic acid molecules (second extended nucleic acid molecules). In some embodiments, the released Round M oligonucleotide comprises a sequence that hybridizes to a Round M splint which in turn hybridizes to the (M−1)th extended nucleic acid molecules in a particular well. In some embodiments, the released Round M oligonucleotide is ligated to the (M−1)th extended nucleic acid molecules using the Round M splint as template to generate the (M)th extended nucleic acid molecules, wherein M is an integer greater than 1.

In some embodiments, the first and/or second wells are formed by a material enclosing a first region and a second region, respectively, on the substrate. In some embodiments, the first splint hybridizes to the first oligonucleotide and the oligonucleotide molecules in the first region. In some embodiments, the first oligonucleotide is not ligated to oligonucleotide molecules in the second region. In some embodiments, the hybridization region between the first splint and the oligonucleotide molecules is at least 3, 4, 5, 6, 7, 8, 9, 10 bp or more than 10 bp. In some embodiments, the hybridization region between the first splint and the first oligonucleotide is at least 3, 4, 5, 6, 7, 8, 9, 10 bp or more than 10 bp.

In some embodiments, the oligonucleotide is ligated using the splint as template without gap filling prior to the ligation. In some embodiments, the oligonucleotide is ligated using the splint as template with gap filling prior to the ligation. In some embodiments, hybridization to the first splint brings the terminal nucleotides of the first oligonucleotide and the oligonucleotide molecules immediately next to each other, and the ligation does not require gap-filling. In some embodiments, hybridization to the first splint brings the terminal nucleotides of the first oligonucleotide and the oligonucleotide molecules next to each other and separated by one or more nucleotides, and the ligation is preceded by gap-filling. In some embodiments, the splint is removed after the ligation.

In some embodiments according to the method for providing an array described herein, the photoresist is a first photoresist. In some embodiment, the first oligonucleotide is ligated to the oligonucleotide molecules in the first region to generate first extended oligonucleotide molecules. In some embodiments, the method further comprises the following steps: (c) applying a second photoresist to the substrate, optionally wherein the second photoresist is applied after the first photoresist is removed from the substrate; (d) irradiating the substrate while the first region is masked and the second region is unmasked, whereby the first or second photoresist in the second region is degraded to render oligonucleotide molecules in the second region available for hybridization and/or ligation, whereas the first extended oligonucleotide molecules in the first region are protected by the second photoresist in the first region from hybridization and/or ligation; and (c) contacting oligonucleotide molecules in the second region with a second splint and a second oligonucleotide comprising a second barcode sequence. In some embodiments, the second splint hybridizes to the second oligonucleotide and the oligonucleotide molecules in the second region. In some embodiments, the second oligonucleotide is ligated to the oligonucleotide molecules in the second region to generate second extended oligonucleotide molecules. In some embodiments, the second oligonucleotide is not ligated to the first extended oligonucleotide molecules in the first region. In some embodiments according to the methods described herein, steps (a)-(b) are part of a first cycle, steps (d)-(e) are part of a second cycle, and steps (a)-(e) are part of a first round, and wherein the method comprises one or more additional rounds. In some embodiments, steps (a)-(e) are part of a first round, the first and second oligonucleotides are Round 1 oligonucleotides, the first and second barcode sequences are Round 1 barcode sequences. In some embodiments, the method further comprises: a′) irradiating the substrate while the first region is unmasked and the second region is masked, whereby a photoresist in the first region is degraded to render the first extended oligonucleotide molecules in the first region available for hybridization and/or ligation, whereas the second extended oligonucleotide molecules in the second region are protected by the photoresist in the second region from hybridization and/or ligation; and (b′) attaching a first Round 2 oligonucleotide comprising a first Round 2 barcode sequence to the first extended oligonucleotide molecules in the first region via hybridization and/or ligation, wherein the second extended oligonucleotide molecules in the second region do not receive the first Round 2 barcode sequence. In some embodiments wherein the photoresist is a first photoresist, and the first Round 2 oligonucleotide is ligated to the first extended oligonucleotide molecules in the first region to generate first further extended oligonucleotide molecules, the method further comprises: (c′) applying a second photoresist to the substrate, optionally wherein the second photoresist is applied after the first photoresist is removed from the substrate; (d′) irradiating the substrate while the first region is masked and the second region is unmasked, whereby the first or second photoresist in the second region is degraded to render the second extended oligonucleotide molecules in the second region available for hybridization and/or ligation, whereas the first further extended oligonucleotide molecules in the first region are protected by the second photoresist in the first region from hybridization and/or ligation; and (e′) attaching a second Round 2 oligonucleotide comprising a second Round 2 barcode sequence to the second extended oligonucleotide molecules in the second region via hybridization and/or ligation, wherein the first further extended oligonucleotide molecules in the first region do not receive the second Round 2 barcode sequence. In some embodiments, the Round 1 barcode sequences are different from each other. In some embodiments, the Round 1 barcode sequences are different from the Round 2 barcode sequences.

In some aspects, provided herein is a method for construction of a hybridization complex or an array comprising nucleic acid molecules and complexes. In some embodiments, provided herein is an oligonucleotide probe for capturing analytes or proxies thereof which may be generated using a method disclosed herein comprising multiple rounds of hybridization and ligation.

In some embodiments, the oligonucleotide probe for capturing analytes or proxies thereof may be generated from an existing array with a ligation strategy. In some embodiments, an array containing a plurality of oligonucleotides (e.g., in situ synthesized oligonucleotides) can be modified to generate a variety of oligonucleotide probes. The oligonucleotides can include various domains such as, spatial barcodes, UMIs, functional domains (e.g., sequencing handle), cleavage domains, and/or ligation handles.

A “spatial barcode” may comprise a contiguous nucleic acid segment or two or more non-contiguous nucleic acid segments that function as a label or identifier that conveys or is capable of conveying spatial information. In some embodiments, a capture probe includes a spatial barcode that possesses a spatial aspect, where the barcode is associated with a particular location within an array or a particular location on a substrate. A spatial barcode can be part of a capture probe on an array generated herein. A spatial barcode can also be a tag attached to an analyte (e.g., a nucleic acid molecule) or a combination of a tag in addition to an endogenous characteristic of the analyte (e.g., size of the analyte or end sequence(s)). A spatial barcode can be unique. In some embodiments where the spatial barcode is unique, the spatial barcode functions both as a spatial barcode and as a unique molecular identifier (UMI), associated with one particular capture probe. Spatial barcodes can have a variety of different formats. For example, spatial barcodes can include polynucleotide spatial barcodes; random nucleic acid and/or amino acid sequences; and synthetic nucleic acid and/or amino acid sequences. In some embodiments, a spatial barcode is attached to an analyte in a reversible or irreversible manner. In some embodiments, a spatial barcode is added to, for example, a fragment of a DNA or RNA sample before, during, and/or after sequencing of the sample. In some embodiments, a spatial barcode allows for identification and/or quantification of individual sequencing-reads. In some embodiments, a spatial barcode is a used as a fluorescent barcode for which fluorescently labeled oligonucleotide probes hybridize to the spatial barcode.

In some embodiments, a spatial array is generated after ligating capture domains (e.g., poly(T) or gene specific capture domains) to the oligonucleotides (e.g., generating capture oligonucleotides). The spatial array can be used with any of the spatial analysis methods described herein. For example, a biological sample can be provided to the generated spatial array. In some embodiments, the biological sample is permeabilized. In some embodiments, the biological sample is permeabilized under conditions sufficient to allow one or more analytes and/or proxies thereof present in the biological sample to interact with the capture probes of the spatial array. After capture of analytes and/or proxies thereof from the biological sample, the analytes and/or proxies thereof can be analyzed (e.g., reverse transcribed or extended, amplified, and sequenced) by any of the variety of methods described herein.

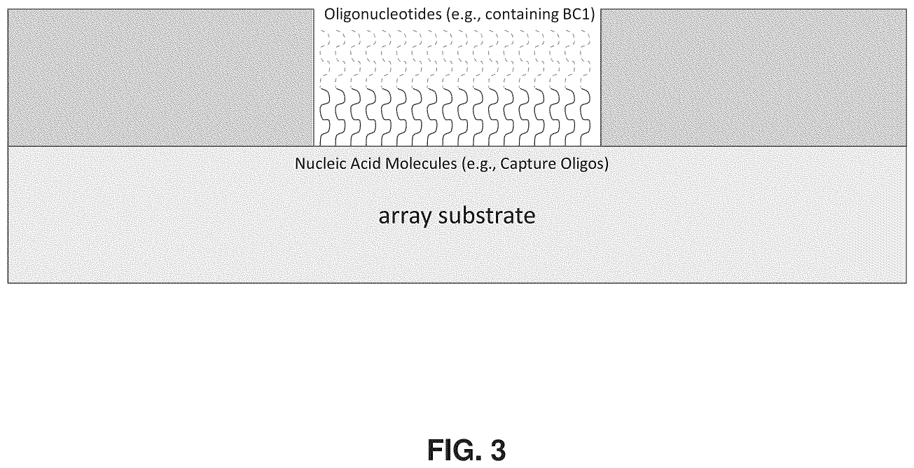

As illustrated in FIG. 3, an oligonucleotide is immobilized on a substrate (e.g., an array) and may comprise a functional sequence such as a primer sequence. In some embodiments, the primer sequence is a sequencing handle that comprises a primer binding site for subsequent processing. The primer sequence can generally be selected for compatibility with any of a variety of different sequencing systems, e.g., 454 Sequencing, Ion Torrent Proton or PGM, Illumina ×10, PacBio, Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Roche 454 sequencing, Ion Torrent Proton or PGM sequencing, Illumina ×10 sequencing, PacBio SMRT sequencing, and Oxford Nanopore sequencing. Further, in some embodiments, functional sequences can be selected for compatibility with other sequencing systems, including non-commercialized sequencing systems.

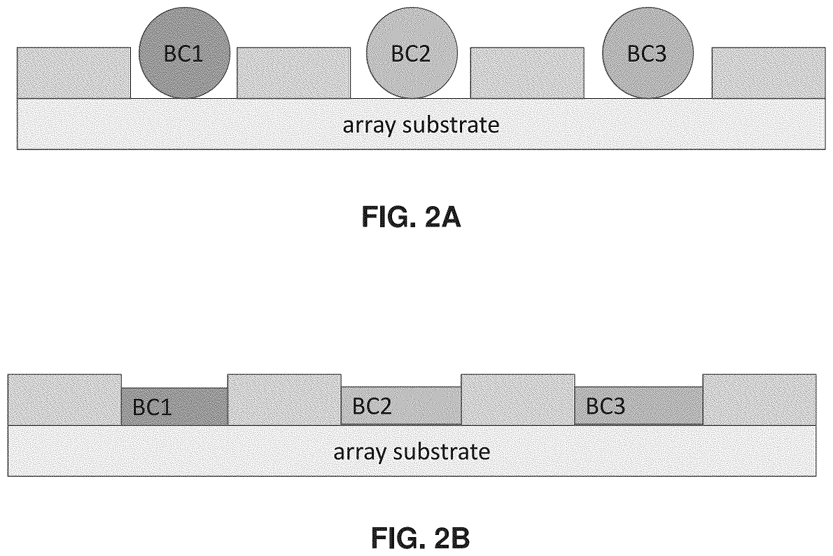

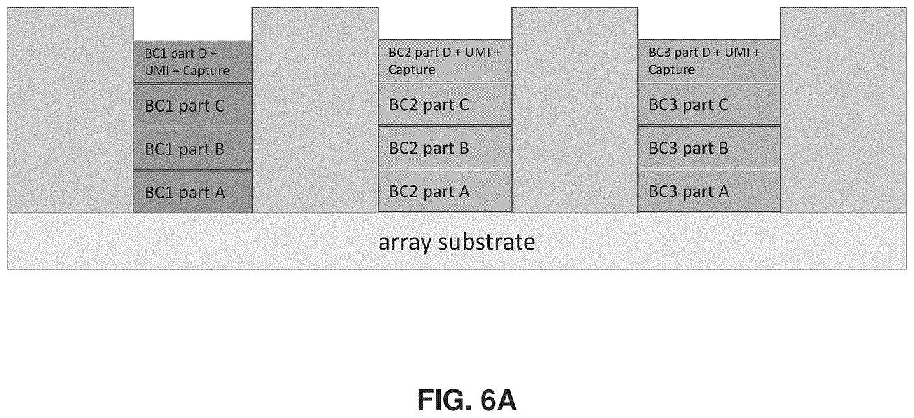

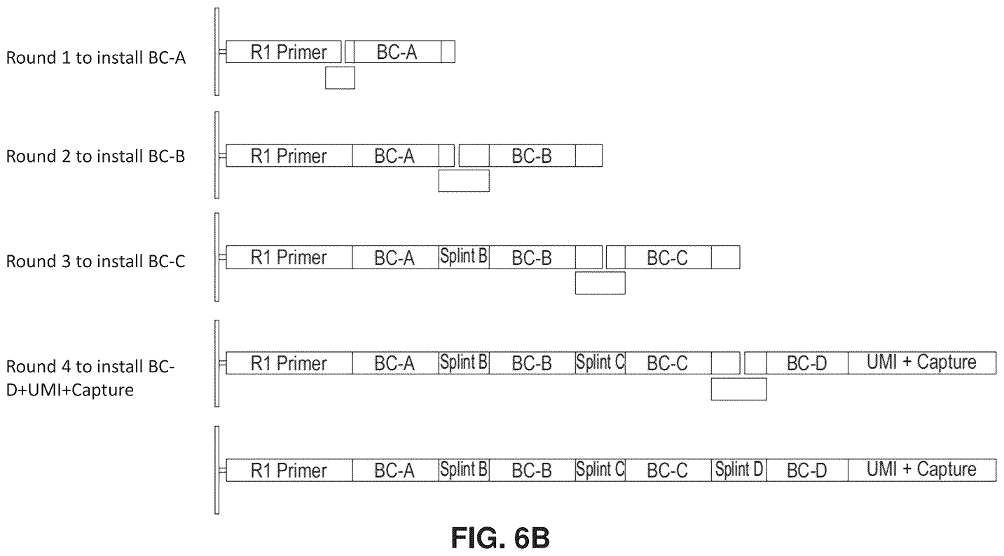

FIGS. 6A-6B show the sequential hybridization/ligation of various domains to generate an oligonucleotide probe for capturing analytes or proxies thereof, by a bead-assisted hybridization/ligation method described herein. FIG. 6A illustrates a method of generating an array by multiple rounds of bead partitioning as in FIGS. 2A-2B to release oligonucleotides comprising parts of barcodes (e.g., BC1 parts A, B, C, and D in one well; BC2 parts A, B, C, and D in one well; and BC3 parts A, B, C, and D in one well; each of the part D portions is also attached to a unique molecular identifier (UMI) and a capture sequence (“Capture”). FIG. 6B shows a graph showing parts of oligonucleotides installed in sequential rounds (Rounds 1 through 4, shown from top to bottom) each comprising multiple cycles of bead-assisted splint hybridization and ligation. The oligonucleotides are shown as the wider rectangles. Round 1 shows installation of an oligonucleotide comprising a first barcode (BC), “A” (BC-A) to an attached oligonucleotide comprising an R1 primer, wherein the primer is attached to the substrate (vertical rectangle). Round 2 shows installation of an oligonucleotide comprising a second barcode, “B” (BC-B). Round 3 shows installation of an oligonucleotide comprising a third barcode, “C” (BC-C). Round D shows installation of an oligonucleotide comprising a fourth barcode, (“D”) (BC-D) and a unique molecular identifier (UMI) and capture sequence. The bottom shows the extended oligonucleotide produced from Rounds 1 through 4. The unlabeled rectangles represent oligonucleotides that facilitate attachment, such as splints, wherein the nucleotide sequences shown between, e.g., BC-A and BC-B, comprise sequences that hybridize to a splint, which is then used as a template to attach BC-B, such that the nucleotide sequence separating BC-A and BC-B, once attached, comprises the sequence “splint B”. Similarly, the portions between BC-B and BC-D comprise the “splint C” sequence once BC-C is attached, and the portions between BC-C and BC-D, comprise the “splint D” sequence once BC-D is attached.

Following a first cycle of bead-assisted hybridization/ligation, an oligonucleotide comprising a part of a barcode (e.g., BC-A) is attached to the oligonucleotide molecule comprising the primer. In some embodiments, the barcode part can be common to all of the oligonucleotide molecules in a given feature. In some embodiments, the barcode part can be different for oligonucleotide molecules in different features. In some embodiments, a splint with a sequence complementary to a portion of the primer of the immobilized oligonucleotide and an additional sequence complementary to a portion of the oligonucleotide comprising BC-A facilitates the ligation of the immobilized oligonucleotide and the oligonucleotide comprising BC-A. In some embodiments, the splint for attaching part BC-A of various sequences to different features is common among the cycles of the same round.

A second cycle of bead-assisted hybridization/ligation involves the addition of another oligonucleotide comprising a part of a barcode (e.g., BC-B) to the immobilized oligonucleotide molecule comprising the primer and BC-A. In some embodiments, a splint with a sequence complementary to a portion of the immobilized oligonucleotide comprising BC-A and an additional sequence complementary to a portion of the oligonucleotide comprising BC-B facilitates the ligation of the oligonucleotide comprising BC-B and the immobilized oligonucleotide comprising BC-A. In some embodiments, the splint for attaching part BC-B of various sequences to different features is common among the cycles of the same round.

A third cycle of bead-assisted hybridization/ligation involves the addition of another oligonucleotide comprising a part of a barcode (e.g., BC-C), added to the immobilized oligonucleotide molecule comprising the primer, BC-A, and BC-B. In some embodiments, a splint with a sequence complementary to a portion of the immobilized oligonucleotide molecule comprising BC-B and an additional sequence complementary to a portion of the oligonucleotide comprising BC-C facilitates the ligation of the immobilized oligonucleotide molecule comprising BC-B and the oligonucleotide comprising BC-C. In some embodiments, the splint for attaching part BC-C of various sequences to different features is common among the cycles of the same round.

A fourth cycle of bead-assisted hybridization/ligation may be performed, which involves the addition of another oligonucleotide comprising a part of a barcode (e.g., BC-D), added to the immobilized oligonucleotide molecule comprising the primer, BC-A, BC-B, and BC-C. In some embodiments, a splint with a sequence complementary to a portion of the immobilized oligonucleotide molecule comprising BC-C and an additional sequence complementary to a portion of the oligonucleotide comprising BC-D facilitates the ligation. In some embodiments, the splint for attaching part BC-D of various sequences to different features is common among the cycles of the same round. In some embodiments, as shown in FIGS. 6A-6B, an oligonucleotide comprising BC-D further comprises a UMI and a capture domain.

In some embodiments, the splint comprises a sequence that is complementary to an oligonucleotide (e.g., an immobilized oligonucleotide), or a portion thereof, and a sequence that is complementary to an oligonucleotide containing a barcode, or a portion thereof. In some embodiments, the splint comprises a sequence that is perfectly complementary (e.g., is 100% complementary) to an oligonucleotide (e.g., an immobilized oligonucleotide), or a portion thereof, and/or a sequence that is perfectly complementary to an oligonucleotide containing a barcode, or a portion thereof. In some embodiments, the splint comprises a sequence that is not perfectly complementary (e.g., is not 100% complementary) to an oligonucleotide (e.g., an immobilized oligonucleotide), or a portion thereof, and/or a sequence that is not perfectly complementary to an oligonucleotide containing a barcode, or a portion thereof. In some embodiments, the splint comprises a sequence that is at least 70%, 75%, 80%, 85%, 90%, 95%, 96%, 97%, 98%, or 99% complementary to an oligonucleotide (e.g., an immobilized oligonucleotide), or a portion thereof, and/or a sequence that is complementary to an oligonucleotide containing a barcode, or a portion thereof. In some embodiments, the splint comprises a sequence that is perfectly complementary (e.g., is 100% complementary) to an oligonucleotide (e.g., an immobilized oligonucleotide), or a portion thereof, but is not perfectly complementary to a sequence that is complementary to an oligonucleotide containing a barcode, or a portion thereof. In some embodiments, the splint comprises a sequence that is not perfectly complementary (e.g., is not 100% complementary) to an oligonucleotide (e.g., an immobilized oligonucleotide), or a portion thereof, but is perfectly complementary to a sequence that is complementary to an oligonucleotide containing a barcode, or a portion thereof. So long as the splint is capable of hybridizing to an oligonucleotide (e.g., an immobilized oligonucleotide), or a portion thereof, and to a sequence that is complementary to an oligonucleotide containing a barcode, or a portion thereof, the splint need not have a sequence that is perfectly complementary to either the oligonucleotide (e.g., the immobilized oligonucleotide) or to the an oligonucleotide containing a barcode.

In some embodiments, oligonucleotides that are exposed and do not receive a ligated oligonucleotide could receive the incorrect barcode during the next cycle or round. In order to prevent generating the wrong barcode at the wrong spot, unligated oligonucleotides may be rendered unavailable for hybridization and/or ligation, e.g., the unligated oligonucleotides can be capped and/or removed. In some embodiments, the oligonucleotides are modified at the 3′. Non-limiting examples of 3′ modifications include dideoxy C-3′ (3′-ddC), 3′ inverted dT, 3′ C3 spacer, 3′ Amino, and 3′ phosphorylation.

In some embodiments according to any of the methods described herein, the method further comprises blocking the 3′ or 5′ termini of barcoded oligonucleotide molecules. In some embodiments, the method further comprises blocking the unligated oligonucleotide molecules in the first region from ligation. In some embodiments, the blocking comprises adding a 3′ dideoxy or a non-ligating 3′ phosphoramidite to the barcoded oligonucleotide molecules. In some embodiments, the blocking comprises adding a 3′ dideoxy or a non-ligating 3′ phosphoramidite to the barcoded oligonucleotide molecules. In some embodiments, the blocking comprises adding a 3′ dideoxy or a non-ligating 3′ phosphoramidite to the unligated oligonucleotide molecules. In some embodiments, the addition is catalyzed by a terminal transferase. In some embodiments, the terminal transferase is TdT. The blocking may be removed after the blocking reaction is completed. In some embodiments, the blocking is removed using an internal digestion of the barcoded oligonucleotide molecules after ligation is completed.

### IV. Compositions, Kits, and Methods of Use

Also provided are compositions produced according to the methods described herein. These compositions include nucleic acid molecules and complexes, such as hybridization complexes, and kits and articles of manufacture (such as arrays) comprising such molecules and complexes.

A. Molecular Arrays

In some aspects, provided herein are arrays, patterned on a surface in situ, for example, by partitioning of beads comprising oligonucleotides into spatially predefined regions (including but not limited to wells), to generate unique DNA sequences in spatial positions in the array. In some embodiments, provided are arrays or compositions generated by any of the methods described above.

In some aspects, provided is an array, wherein the method of providing the array comprises: (a) partitioning a plurality of beads into wells on a substrate, wherein the bead in each well each comprises a different oligonucleotide; and (b) disrupting the beads to release the oligonucleotides, wherein the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the oligonucleotides comprised in the beads comprise barcode sequences. In some embodiments, the method of generating the array comprises one or more steps of partitioning pluralities of beads that enable barcodes to be generated combinatorially, for example, in as few as three rounds of assembly. The process can be repeated N cycles (each cycle for one or more partitions and disruptions of beads) for Round 1 until all desired wells have received partitioned beads and/or release of oligonucleotides following disruptions of beads. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round M barcode (which may be the same or different for molecules in any two given features) to each of the growing oligonucleotides in the features.

In some embodiments, provided is an array, wherein the method of providing the array comprises: (a) partitioning a plurality of Round 1 beads into wells on a substrate, wherein the Round 1 bead in a first well and the Round 1 bead in a second well each comprises a different Round 1 oligonucleotide; and (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules, thereby providing on the substrate an array comprising the extended nucleic acid molecules.

In some embodiments, the array is generated by a process comprising partitioning beads into at least the first well and the second well. In some embodiments, the first and/or second wells are formed by a material enclosing a first region and a second region, respectively, on the substrate. In some embodiments, the first and/or second wells are formed by etching a layer of the material on the substrate on the substrate. In some embodiments, the one or more wells are formed by etching a layer of the material on the substrate.

In some embodiments, the nucleic acids are provided and/or immobilized in one or more wells before or after the etching step. In some embodiments, the nucleic acid molecules in the one or more wells comprise the same sequence. In some embodiments, the nucleic acid molecules in the first well and the nucleic acid molecules in the second well comprise the same sequence. In some embodiments, the nucleic acid molecules are universal among wells on the substrate.

In some embodiments, the nucleic acid molecules in each of the plurality of wells are different in sequences. In some embodiments, the nucleic acid molecules in the first well and those in the second well are different in sequences. In some embodiments, the nucleic acid in at least about any one of: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the plurality of wells are unique in sequences. In some embodiments, the nucleic acid in each of the plurality of wells are unique in sequences.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round M beads each comprises a unique Round M oligonucleotide, wherein M is an integer that is 1 or greater. In some embodiments, at least about 90% of Round M beads each comprises a unique Round M oligonucleotide, wherein M is an integer that is 1 or greater. In some embodiments, each Round M bead comprises a unique Round M oligonucleotide, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 1 beads each comprises a unique Round 1 oligonucleotide. In some embodiments, at least about 90% of Round 1 beads each comprise a unique Round 1 oligonucleotide. In some embodiments, each Round 1 bead comprises a unique Round 1 oligonucleotide.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives one Round M bead, wherein M is an integer that is 1 or greater. In some embodiments, at least about 90% of wells each receives one Round M bead, wherein M is an integer that is 1 or greater. In some embodiments, each well receives one Round M bead, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives one Round 1 bead. In some embodiments, at least about 90% of wells each receives one Round 1 bead. In some embodiments, each well receives one Round 1 bead.

In some embodiments, the Round M oligonucleotides are at least about any one of 3, 4, 5, 6, 7, 8, 9, 10, 12, 14, 16, 18, 20, 25, 30, 35, 40, 45, or 50 nucleotides in length, wherein M is an integer that is 1 or greater. In some embodiments, the Round M oligonucleotides are at least about 4 nucleotides in length, wherein M is an integer that is 1 or greater. In some embodiments, the Round 1 oligonucleotides are at least about any one of 3, 4, 5, 6, 7, 8, 9, 10, 12, 14, 16, 18, 20, 25, 30, 35, 40, 45, or 50 nucleotides in length. In some embodiments, the Round 1 oligonucleotides are at least about 4 nucleotides in length.

In some embodiments, the released Round 1 oligonucleotides in the plurality of wells comprise a plurality of corresponding Round 1 barcode sequences, respectively, wherein each of the plurality of Round 1 barcode sequences are different. In some embodiments, the released Round 1 oligonucleotides in the first and second wells comprise a first Round 1 barcode sequence and a second Round 1 barcode sequence, respectively, wherein the first and second Round 1 barcode sequences are different.

In some embodiments, the released Round 1 oligonucleotide comprises a sequence that hybridizes to a Round 1 splint which in turn hybridizes to the nucleic acid molecules in a particular well. In some embodiments, the released Round 1 oligonucleotide is ligated to the nucleic acid molecules using the Round 1 splint as template to generate the extended nucleic acid molecules.

In some embodiments, the sequence that hybridizes to the Round 1 splint is common between Round 1 oligonucleotides in different wells. In some embodiments, the Round 1 splint is comprised in the Round 1 bead and released upon disruption of the bead. In some embodiments, the Round 1 splint is not comprised in the Round 1 bead and is separately delivered to the wells.

In some embodiments, the process of generating the array is repeated for M rounds, wherein M is an integer that is 1 or greater. In some embodiments, the sequence that hybridizes to the Round M splint is common between Round M oligonucleotides in different wells. In some embodiments, the Round M splint is comprised in the Round M bead and released upon disruption of the bead. In some embodiments, the Round M splint is not comprised in the Round M bead and is separately delivered to the wells.

In some embodiments, wherein the process of generating the array is repeated for M rounds, wherein the released Round M oligonucleotide comprises a sequence that hybridizes to a Round M+1 splint which in turn hybridizes to a Round M+1 oligonucleotide, optionally wherein the Round M+1 oligonucleotide is ligated to the extended nucleic acid molecules using the Round M+1 splint as template to generate further extended nucleic acid molecules. In some embodiments, the sequence that hybridizes to the Round M+1 splint is common between Round M oligonucleotides in different wells. In some embodiments, the Round M+1 splint is common between different wells.

In some embodiments, the released Round 1 oligonucleotide comprises a sequence that hybridizes to a Round 2 splint which in turn hybridizes to a Round 2 oligonucleotide, optionally wherein the Round 2 oligonucleotide is ligated to the extended nucleic acid molecules using the Round 2 splint as template to generate further extended nucleic acid molecules. In some embodiments, the sequence that hybridizes to the Round 2 splint is common between Round 1 oligonucleotides in different wells. In some embodiments, the Round 2 splint is common between different wells.

In some embodiments, the hybridization region between the Round 1 splint and nucleic acid molecule is at least 3, 4, 5, 6, 7, 8, 9, 10 bp or more than 10 bp, and/or wherein the hybridization region between the Round 1 splint and the Round 1 oligonucleotide is at least 3, 4, 5, 6, 7, 8, 9, 10 bp or more than 10 bp. In some embodiments, the hybridization region between the Round 2 splint and the Round 1 oligonucleotide is at least 3, 4, 5, 6, 7, 8, 9, 10 bp or more than 10 bp, and/or wherein the hybridization region between the Round 2 splint and the Round 2 oligonucleotide is at least 3, 4, 5, 6, 7, 8, 9, 10 bp or more than 10 bp.

In some embodiments, the hybridization region between the Round M+1 splint and the Round M oligonucleotide is at least 3, 4, 5, 6, 7, 8, 9, 10 bp or more than 10 bp, and/or wherein the hybridization region between the Round M+1 splint and the Round M+1 oligonucleotide is at least 3, 4, 5, 6, 7, 8, 9, 10 bp or more than 10 bp, wherein M is an integer that is 1 or greater.

In some aspects, provided herein are arrays patterned on a surface in situ, for example, by one or more rounds of partitioning of beads comprising oligonucleotides into spatially predefined regions (including but not limited to wells), to generate unique combinatorial DNA sequences in spatial positions in the array. In some embodiments, provided are arrays or compositions generated by any of the methods described above.

In some aspects, provided is an array, wherein the method of providing the array comprises: (a) partitioning a plurality of beads into wells on a substrate, wherein the bead in each well each comprises a different oligonucleotide; and (b) disrupting the beads to release the oligonucleotides, wherein the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the oligonucleotides comprised in the beads comprise barcode sequences. In some embodiments, the method of generating the array comprises one or more steps of partitioning pluralities of beads that enable barcodes to be generated combinatorially, for example, in as few as three rounds of assembly. The process can be repeated N cycles (each cycle for one or more partitions and disruptions of beads) for Round 1 until all desired wells have received partitioned beads and/or release of oligonucleotides following disruptions of beads. The process can be repeated M rounds to achieve a desired barcode diversity, for example, by attaching a round 2 barcode (which may be the same or different for molecules in any two given features), a round 3 barcode (which may be the same or different for molecules in any two given features), . . . , and a round M barcode (which may be the same or different for molecules in any two given features) to each of the growing oligonucleotides in the well.

In some embodiments, provided is an array, wherein the method of providing the array comprises: (a) partitioning a plurality of Round 1 beads into a plurality of wells on a substrate, wherein the Round 1 beads in each of the plurality of wells each comprises a different Round 1 oligonucleotide; (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules, (c) partitioning a plurality of Round 2 beads into the plurality of wells on the substrate, wherein Round 2 beads in each of the plurality of wells each comprises a different Round 2 oligonucleotide; and (d) disrupting the Round 2 beads to release the Round 2 oligonucleotides, wherein the released Round 2 oligonucleotides are attached to the extended nucleic acid molecules in the corresponding well via hybridization and/or ligation to generate further extended nucleic acid molecules (or the second extended nucleic acid molecules). In some embodiments, the method further comprises the step of: (e) partitioning a plurality of Round M beads into wells on a substrate, wherein the Round M beads in each of the plurality of wells each comprises a different Round M oligonucleotide; (f) disrupting the Round M beads to release the Round M oligonucleotides, wherein the released Round M oligonucleotides are attached to (M−1)th extended nucleic acid molecules in the corresponding well via hybridization and/or ligation to generate (M)th extended nucleic acid molecules; (g) partitioning a plurality of Round M+1 beads into wells on a substrate, wherein the Round M+1 beads in each of the plurality of wells each comprises a different Round M+1 oligonucleotide; (f) disrupting the Round M+1 beads to release the Round M+1 oligonucleotides, wherein the released Round M+1 oligonucleotides are attached to (M)th extended nucleic acid molecules in the corresponding well via hybridization and/or ligation to generate (M+1)th extended nucleic acid molecules, wherein M is an integer that is greater than 2, optionally wherein the step of (e), (f), (g), (h) are repeated N times, wherein M is incremented by 2 in each repetition, wherein N is an integer that is about 2, 3, 4, 5, 6, 7, 8, 9, 10, 15, 20, 25, 30, 35, 40, 50, or greater.

In some embodiments, provided is an array, wherein the method of providing the array comprises: (a) partitioning a plurality of Round 1 beads into wells on a substrate, wherein the Round 1 bead in a first well and the Round 1 bead in a second well each comprises a different Round 1 oligonucleotide; (b) disrupting the Round 1 beads to release the Round 1 oligonucleotides, wherein the released Round 1 oligonucleotides are attached to nucleic acid molecules in the corresponding well via hybridization and/or ligation to generate extended nucleic acid molecules (or the first extended molecules), (c) partitioning a plurality of Round 2 beads into wells on the substrate, wherein the Round 2 bead in the first well and the Round 2 bead in the second well each comprises a different Round 2 oligonucleotide; and (d) disrupting the Round 2 beads to release the Round 2 oligonucleotides, wherein the released Round 2 oligonucleotides are attached to the extended nucleic acid molecules in the corresponding well via hybridization and/or ligation to generate further extended nucleic acid molecules (or the second extended nucleic acid molecules). In some embodiments, wherein the method can be repeated for M rounds, wherein M is an integer that is greater than 2. In some embodiments, the method further comprises the step of: (e) partitioning a plurality of Round M beads into wells on a substrate, wherein the Round M bead in a first well and the Round M bead in a second well each comprises a different Round M oligonucleotide; (f) disrupting the Round M beads to release the Round M oligonucleotides, wherein the released Round M oligonucleotides are attached to (M−1)th extended nucleic acid molecules in the corresponding well via hybridization and/or ligation to generate (M)th extended nucleic acid molecules; (g) partitioning a plurality of Round M+1 beads into wells on a substrate, wherein the Round M+1 bead in a first well and the Round M+1 bead in a second well each comprises a different Round M+1 oligonucleotide; (f) disrupting the Round M+1 beads to release the Round M+1 oligonucleotides, wherein the released Round M+1 oligonucleotides are attached to (M)th extended nucleic acid molecules in the corresponding well via hybridization and/or ligation to generate (M+1)th extended nucleic acid molecules, wherein M is an integer that is greater than 2, optionally wherein the step of (e), (f), (g), and (h) are repeated N times, wherein M is incremented by 2 in each repetition, wherein N is 2, 3, 4, 5, 6, 7, 8, 9, 10, 15, 20, 25, 30, 35, 40, 50, or higher.

In some embodiments, the method comprises partitioning beads into at least the first well and the second well. In some embodiments, the first and/or second wells are formed by a material enclosing a first region and a second region, respectively, on the substrate. In some embodiments, the first and/or second wells are formed by etching a layer of the material on the substrate on the substrate. In some embodiments, the one or more wells are formed by etching a layer of the material on the substrate.

In some embodiments, the nucleic acids are provided and/or immobilized in one or more wells before or after the etching step. In some embodiments, the nucleic acid molecules in the one or more wells comprise the same sequence. In some embodiments, the nucleic acid molecules immobilized in the first well and the nucleic acid molecules in the second well comprise the same sequence. In some embodiments, the nucleic acid molecules are universal among wells on the substrate.

In some embodiments, the nucleic acid molecules in each of the plurality of wells are different in sequences. In some embodiments, the nucleic acid molecules in the first well and those in the second well are different in sequences. In some embodiments, the nucleic acid molecules in at least about any one of: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of the plurality of wells are unique in sequences. In some embodiments, the nucleic acid molecules each of the plurality of wells are unique in sequences.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round M beads each comprises a unique Round M oligonucleotide, wherein M is an integer that is 1 or greater. In some embodiments, at least about 90% of Round M beads each comprises a unique Round M oligonucleotide, wherein M is an integer that is 1 or greater. In some embodiments, each Round M bead comprises a unique Round M oligonucleotide, wherein M is an integer that is 1 or greater. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 1 beads each comprises a unique Round 1 oligonucleotide. In some embodiments, at least about 90% of Round 1 beads each comprise a unique Round 1 oligonucleotide. In some embodiments, each Round 1 bead comprises a unique Round 1 oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of Round 2 beads each comprises a unique Round 2 oligonucleotide. In some embodiments, at least about 90% of Round 2 beads each comprise a unique Round 2 oligonucleotide. In some embodiments, each Round 2 bead comprises a unique Round 2 oligonucleotide.

In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives one Round M bead, wherein M is an integer that is 1 or greater. In some embodiments, at least about 90% of wells each receives one Round M bead, wherein M is an integer that is 1 or greater. In some embodiments, each well receives one Round M bead, wherein M is an integer that is 1 or greater. In some embodiments, each Round M bead comprises a unique Round M oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives one Round 1 bead. In some embodiments, at least about 90% of wells each receives one Round 1 bead. In some embodiments, each well receives one Round 1 bead. In some embodiments, each Round 1 bead comprises a unique Round 1 oligonucleotide. In some embodiments, at least about any one of 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, or 99% of wells each receives one Round 2 bead. In some embodiments, at least about 90% of wells each receives one Round 2 bead. In some embodiments, each well receives one Round 2 bead. In some embodiments, each Round 2 bead comprises a unique Round 2 oligonucleotide.

In some embodiments, the Round M oligonucleotides are at least about any one 3, 4, 5, 6, 7, 8, 9, 10, 12, 14, 16, 18, 20, 25, 30, 35, 40, 45, or 50 nucleotides in length, wherein M is an integer that is 1 or greater. In some embodiments, the Round M oligonucleotides are at least about 4 nucleotides in length, wherein M is an integer that is 1 or greater. In some embodiments, the Round 1 oligonucleotides are at least about any one 3, 4, 5, 6, 7, 8, 9, 10, 12, 14, 16, 18, 20, 25, 30, 35, 40, 45, or 50 nucleotides in length. In some embodiments, the Round 1 oligonucleotides are at least about 4 nucleotides in length. In some embodiments, the Round 2 oligonucleotides are at least about any one 3, 4, 5, 6, 7, 8, 9, 10, 12, 14, 16, 18, 20, 25, 30, 35, 40, 45, or 50 nucleotides in length. In some embodiments, the Round 2 oligonucleotides are at least about 4 nucleotides in length.

In some embodiments, the released Round 1 oligonucleotides in the plurality of wells comprise a plurality of corresponding Round 1 barcode sequences, respectively, wherein each of the plurality of Round 1 barcode sequences are different. In some embodiments, the released Round 1 oligonucleotides in the first and second wells comprise a first Round 1 barcode sequence and a second Round 1 barcode sequence, respectively, wherein the first and second Round 1 barcode sequences are different. In some embodiments, the released Round 2 oligonucleotides in the plurality of wells comprise a plurality of corresponding Round 2 barcode sequences, respectively, wherein each of the plurality of Round 2 barcode sequences are different. In some embodiments, the released Round 2 oligonucleotides in the first and second wells comprise a first Round 2 barcode sequence and a second Round 2 barcode sequence, respectively, wherein the first and second Round 2 barcode sequences are different. In some embodiments, the released Round M oligonucleotides in the plurality of wells comprise a plurality of corresponding Round M barcode sequences, respectively, wherein each of the plurality of Round M barcode sequences are different. In some embodiments, the released Round M oligonucleotides in the first and second wells comprise a first Round M barcode sequence and a second Round M barcode sequence, respectively, wherein the first and second Round M barcode sequences are different, wherein M is an integer that is 1 or greater.

In some embodiments, the released Round 1 oligonucleotide comprises a sequence that hybridizes to a Round 1 splint which in turn hybridizes to the nucleic acid molecules in a particular well. In some embodiments, the released Round 1 oligonucleotide is ligated to the nucleic acid molecules using the Round 1 splint as template to generate the (first) extended nucleic acid molecules. In some embodiments, the released Round 2 oligonucleotide comprises a sequence that hybridizes to a Round 2 splint which in turn hybridizes to the (first) extended nucleic acid molecules in a particular well. In some embodiments, the released Round 2 oligonucleotide is ligated to the (first) extended nucleic acid molecules using the Round 2 splint as template to generate the further extended nucleic acid molecules (second extended nucleic acid molecules). In some embodiments, the released Round M oligonucleotide comprises a sequence that hybridizes to a Round M splint which in turn hybridizes to the (M−1)th extended nucleic acid molecules in a particular well. In some embodiments, the released Round M oligonucleotide is ligated to the (M−1)th extended nucleic acid molecules using the Round M splint as template to generate the (M)th extended nucleic acid molecules, wherein M is an integer greater than 1.

In some embodiments, the sequence that hybridizes to the Round 1 splint is common between Round 1 oligonucleotides in different wells. In some embodiments, the Round 1 splint is comprised in the Round 1 bead and released upon disruption of the bead. In some embodiments, the Round 1 splint is not comprised in the Round 1 bead and is separately delivered to the wells. In some embodiments, the sequence that hybridizes to the Round 2 splint is common between Round 2 oligonucleotides in different wells. In some embodiments, the Round 2 splint is comprised in the Round 2 bead and released upon disruption of the bead. In some embodiments, the Round 2 splint is not comprised in the Round 2 bead and is separately delivered to the wells. In some embodiments, the sequence that hybridizes to the Round 3 splint is common between Round 3 oligonucleotides in different wells. In some embodiments, the Round 3 splint is comprised in the Round 3 bead and released upon disruption of the bead. In some embodiments, the Round 3 splint is not comprised in the Round 3 bead and is separately delivered to the wells.

In some embodiments, the method is repeated for M rounds, wherein M is an integer that is 1 or greater. In some embodiments, the sequence that hybridizes to the Round M splint is common between Round M oligonucleotides in different wells. In some embodiments, the Round M splint is comprised in the Round M bead and released upon disruption of the bead. In some embodiments, the Round M splint is not comprised in the Round M bead and is separately delivered to the wells.

In some embodiments, wherein the method is repeated for M rounds, wherein the released Round M oligonucleotide comprises a sequence that hybridizes to a Round M+1 splint which in turn hybridizes to a Round M+1 oligonucleotide, optionally wherein the Round M+1 oligonucleotide is ligated to the (M)th extended nucleic acid molecules using the Round M+1 splint as template to generate (M+1)th extended nucleic acid molecules. In some embodiments, the sequence that hybridizes to the Round M+1 splint is common between Round M+1 oligonucleotides in different wells. In some embodiments, the Round M+1 splint is common between different wells.

In some embodiments, the released Round 1 oligonucleotide comprises a sequence that hybridizes to a Round 2 splint which in turn hybridizes to a Round 2 oligonucleotide, optionally wherein the Round 2 oligonucleotide is ligated to the (first) extended nucleic acid molecules using the Round 2 splint as template to generate further extended nucleic acid molecules (e.g. second extended nucleic acid molecules). In some embodiments, the sequence that hybridizes to the Round 2 splint is common between Round 1 oligonucleotides in different wells. In some embodiments, the Round 2 splint is common between different wells.

In some embodiments, the released Round 2 oligonucleotide comprises a sequence that hybridizes to a Round 3 splint which in turn hybridizes to a Round 3 oligonucleotide, optionally wherein the Round 3 oligonucleotide is ligated to the further extended nucleic acid molecules (e.g. second extended nucleic acid molecules) using the Round 3 splint as template to generate even further extended nucleic acid molecules (e.g. third extended nucleic acid molecules). In some embodiments, the sequence that hybridizes to the Round 3 splint is common between Round 2 oligonucleotides in different wells. In some embodiments, the Round 3 splint is common between different wells.

In some embodiments, the hybridization region between the Round 1 splint and nucleic acid molecule is at least 3, 4, 5, 6, 7, 8, 9, 10 bp or more than 10 bp, and/or wherein the hybridization region between the Round 1 splint and the Round 1 oligonucleotide is at least 3, 4, 5, 6, 7, 8, 9, 10 bp or more than 10 bp. In some embodiments, the hybridization region between the Round 2 splint and the Round 1 oligonucleotide is at least 3, 4, 5, 6, 7, 8, 9, 10 bp or more than 10 bp, and/or wherein the hybridization region between the Round 2 splint and the Round 2 oligonucleotide is at least 3, 4, 5, 6, 7, 8, 9, 10 bp or more than 10 bp. In some embodiments, the hybridization region between the Round 3 splint and the Round 2 oligonucleotide is at least 3, 4, 5, 6, 7, 8, 9, 10 bp or more than 10 bp, and/or wherein the hybridization region between the Round 3 splint and the Round 3 oligonucleotide is at least 3, 4, 5, 6, 7, 8, 9, 10 bp or more than 10 bp.

In some embodiments, the hybridization region between the Round M+1 splint and the Round M oligonucleotide is at least 3, 4, 5, 6, 7, 8, 9, 10 bp or more than 10 bp, and/or wherein the hybridization region between the Round M+1 splint and the Round M+1 oligonucleotide is at least 3, 4, 5, 6, 7, 8, 9, 10 bp or more than 10 bp, wherein M is an integer that is 1 or greater.

In some embodiments, the Round 1 oligonucleotide, the Round 2 oligonucleotide, and/or the Round 3 oligonucleotide each comprises a unique molecular identifier (UMI) sequence. In some embodiments, each of the oligonucleotides of M rounds (e.g. Rounds 1, 2, 3, 4, 5 . . . up to M) comprises a unique molecular identifier (UMI) sequence, wherein M is an integer that is greater than 1.

In some embodiments, the Round 1 oligonucleotide, the Round 2 oligonucleotide, and/or the Round 3 oligonucleotide each comprises a capture sequence. In some embodiments, each of the oligonucleotides of M rounds (e.g. Rounds 1, 2, 3, 4, 5 . . . up to M) comprises a capture sequence, wherein M is an integer that is greater than 1. In some embodiments, the capture sequence. In some embodiments, the capture sequence is poly(dT).

In some embodiments, the Round 1 oligonucleotide, the Round 2 oligonucleotide, and/or the Round 3 oligonucleotide each comprises a different capture sequence. In some embodiments, each of all oligonucleotides from M rounds (e.g. Rounds 1, 2, 3, 4, 5 . . . up to M) comprises a different capture sequence, wherein M is an integer that is greater than 1. In some embodiments, each capture sequence is designed to couple to one or more analytes. In some embodiments, each capture sequence is designed to couple to different analytes.

In some embodiments, the analyte is a cell surface protein. In some embodiments, the analyte is a secreted analyte. In some embodiments, the analyte is a secreted protein. In some embodiments, the cell surface protein comprises a proteoglycan and/or a glycoprotein.

In some embodiments, the capture sequence comprised in the Round 1 oligonucleotides is designed to couple to a cell surface protein. In some embodiments, the capture sequence comprised in the Round 1 oligonucleotides is designed to couple to a secreted analyte. In some embodiments, the capture sequence comprised in the Round 2 oligonucleotides is designed to couple to a cell surface protein. In some embodiments, the capture sequence comprised in the Round 2 oligonucleotides is designed to couple to a secreted analyte. In some embodiments, the capture sequence comprised in the Round 3 oligonucleotides is designed to couple to a cell surface protein. In some embodiments, the capture sequence comprised in the Round 3 oligonucleotides is designed to couple to a secreted analyte. In some embodiments, the capture sequence comprised in the Round M oligonucleotides is designed to couple to a cell surface protein. In some embodiments, the capture sequence comprised in the Round M oligonucleotides is designed to couple to a secreted analyte.

In some embodiments, the Round 1 oligonucleotide comprises a capture sequence designed to couple to a first cell surface protein; the Round 2 oligonucleotide comprises a capture sequence designed to couple to a second cell surface protein; and/or the Round 3 oligonucleotide comprises a capture sequence designed to couple to a first secreted analyte.

B. Master and Copy Arrays

In some aspects, provided herein are master arrays patterned a surface in situ, for example, by one or more rounds of partitioning of beads comprising oligonucleotides into spatially predefined regions (including but not limited to wells), to generate unique DNA sequences in spatial positions in the array, wherein copy arrays can be generated from the master array by migration of the unique DNA sequences. In some embodiments, provided are arrays or compositions generated by any of the methods described above, optionally wherein the arrays or compositions comprise a master array and/or copy arrays.

In some embodiments according to any one of the arrays described above, the array on the substrate comprises extended nucleic acid molecules, optionally wherein the array on the substrate is a master array. In some embodiments, the array on the substrate comprises the extended nucleic acid molecules, the further extended nucleic acid molecules and/or the even further extended nucleic acid molecules. In some embodiments, the array on the substrate comprises the first extended nucleic acid molecules, the second extended nucleic acid molecules and/or the third extended nucleic acid molecules. In some embodiments, the array on the substrate comprises the first, second, third, fourth, . . . , and/or up to (M)th extended nucleic acid molecules, wherein M is an integer that is greater than 1.

In some embodiments, the extended nucleic acid molecules, the further extended nucleic acid molecules and/or the even further extended nucleic acid molecules are migrated or allowed to migrate into a porous material abutting the well. In some embodiments, the extended nucleic acid molecules, the first extended nucleic acid molecules, the second extended nucleic acid molecules and/or the third extended nucleic acid molecules are migrated or allowed to migrate into a porous material abutting the well. In some embodiments, the one or more of the first, second, third, fourth, . . . , and/or up to (M)th extended nucleic acid molecules are migrated or allowed to migrate into a porous material abutting the well.

In some embodiments according to any one of the methods of providing arrays described above, wherein the extended nucleic acid molecules, the further extended nucleic acid molecules, or the even further extended nucleic acid molecules, optionally after cleavage from the substrate and/or amplification (e.g., by PCR), are migrated or allowed to migrate into a porous material abutting the wells.

In some embodiments, provided herein is composition comprising a master array and copy arrays, wherein the method of providing the master array and copy arrays comprises: (a) partitioning a plurality of beads into wells on a substrate, wherein the bead in a first well and the bead in a second well each comprises a different oligonucleotide; (b) disrupting the plurality of beads to release the oligonucleotides, wherein the released oligonucleotides are migrated or allowed to migrate into a porous material abutting the wells, wherein the migration paths in a porous material are substantially parallel to each other; and (c) dividing the porous material into subparts along one or more planes intersecting the migration paths, thereby generating copies of an the array comprising the released oligonucleotides.

In some embodiments, wherein before the migration or the allowance to migrate, the extended nucleic acid molecules, the further extended nucleic acid molecules, and/or the even further extended nucleic acid molecules are subjected to cleavage from the substrate and/or amplification. In some embodiments, wherein before the migration or the allowance to migrate, first extended nucleic acid molecules, the second extended nucleic acid molecules and/or the third extended nucleic acid molecules are subjected to cleavage from the substrate and/or amplification. In some embodiments, wherein before the migration or the allowance to migrate, the first, second, third, fourth, . . . , and/or up to (M)th extended nucleic acid molecules are subjected to cleavage from the substrate and/or amplification.

In some embodiments, the porous material is a polymer matrix. In some embodiments, the porous material is a silica matrix. In some embodiments, the porous material is a gel. In some embodiments, the porous material is a hydrogel. In some embodiments, the porous material is a polymer matrix comprising collagen, laminin and/or fibronectin. In some embodiments, the porous material is a polymer matrix comprising hydrogel. In some embodiments, the porous material is a polymer matrix comprising polyacrylamide.

In some embodiments, the migration comprises migration along a field, such as but not limited to migration along an electromagnetic field. In some embodiments, an electromagnetic field is applied across the medium to migrate the nucleic acids and/or oligonucleotides through the medium. In some embodiments, the migration comprises electrophoresis.

In some embodiments, the migration paths in the porous materials are substantially parallel to one another, and the porous material is divided into subparts along one or more planes intersecting the migration paths, thereby generating copies of the array. In some embodiments, any two migration paths in the porous materials vary in direction by no more than about any one of 20°, 10°, 9°, 8°, 7° 6°, 5° 4° 3° 2°, 10, 0.5°, 0.1° or 0.01° angle. In some embodiments, the porous material is divided into subparts along one or more planes that are substantially perpendicular to the mean migration direction. In some embodiments the porous material is divided into subparts along one or more planes that are at about any one of 170°, 1710, 172°, 173°, 174°, 175°, 176°, 177°, 178°, 179°, 180°, 181°, 182°, 183°, 184°, 185°, 186°, 187°, 188°, 189°, or 190° degree to the mean migration direction.

In some embodiments, each of the subparts is divided along one or more planes, wherein a subpart is about any one of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 8 mm, or 10 mm in thickness. In some embodiments, the subpart is about any one of 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1, 1 to 2, 2 to 3, 3 to 4, 4 to 5, 5 to 8, or 8 to 10 mm in thickness. In some embodiments, each of the subparts is divided along one or more planes, wherein each subpart is about any one of 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 8 mm, or 10 mm in thickness. In some embodiments, each subpart is about any one of 0.1 to 0.2, 0.2 to 0.3, 0.3 to 0.4, 0.4 to 0.5, 0.5 to 0.6, 0.6 to 0.7, 0.7 to 0.8, 0.8 to 0.9, 0.9 to 1, 1 to 2, 2 to 3, 3 to 4, 4 to 5, 5 to 8, or 8 to 10 mm in thickness.

C. Three-Dimensional Spatial Array

In some aspects, provided herein are three-dimensional arrays patterned on a surface, for example, by one or more rounds of partitioning of beads comprising oligonucleotides into spatially predefined regions (including but not limited to wells), to generate unique DNA sequences in spatial positions in the array, wherein the unique DNA sequences in the array are further migrated. In some embodiments, provided are arrays generated by any of the methods described above, optionally wherein the array is a three-dimensional spatial array.

In some embodiments, provided is a three-dimensional array (3D-array), wherein the method of providing the 3D-array comprises: (a) partitioning a plurality of beads into wells on a substrate, wherein a first bead in a first well comprises a plurality of first oligonucleotides of varying lengths which comprise the same first barcode sequence, and a second bead in a second well comprises a plurality of second oligonucleotides of varying lengths which comprise the same second barcode sequence; and (b) disrupting the first and second beads to release the oligonucleotides in the corresponding well. In some embodiments, the released oligonucleotides are attached to nucleic acid molecules in the corresponding well via hybridization and/or ligation to generate extended nucleic acid molecules, thereby providing on the substrate an array comprising the extended nucleic acid molecules. In some embodiments, the process of generating the 3D array further comprises cleaving the extended nucleic acid molecules from the substrate, thereby releasing the extended nucleic acid molecules from the substrate. In some embodiments, the released oligonucleotides, or the released extended nucleic acid molecules are migrated or allowed to migrate in a direction into a porous material abutting the wells, wherein the position of a particular released oligonucleotide in the direction correlates with the length of the particular released oligonucleotide, thereby providing in the porous material a 3D-array comprising the released oligonucleotides or released extended nucleic acid molecules.

In some embodiments, a three-dimensional array (3D-array), wherein the process of generating the 3D array of providing the 3D-array comprises: (a) partitioning a plurality of beads into wells on a substrate, wherein a first bead in a first well comprises a plurality of first oligonucleotides of varying lengths which comprise the same first barcode sequence, and a second bead in a second well comprises a plurality of second oligonucleotides of varying lengths which comprise the same second barcode sequence; and (b) disrupting the first and second beads to release the oligonucleotides in the corresponding well, wherein the released oligonucleotides are migrated or allowed to migrate in a direction into a porous material abutting the wells, wherein the position of a particular released oligonucleotide in the direction correlates with the length of the particular released oligonucleotide. In some embodiments, the porous material is a three-dimensional medium.

In some embodiments, the first and second wells are comprised within a plurality of N wells, wherein Nis an integer and wherein N has a numerical value greater than any one of 5, 10, 50, 100, 200, 500, 1000, 10000, 20000, 50000, 100000. In some embodiments, the first and second wells are comprised within a plurality of about any one of 1×10¹, 5×10¹, 1×10², 5×10², 1×10³, 5×10³, 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹, 5×10¹¹, 1×10¹², 5×10¹², 1×10¹³, 5×10¹³, 1×10¹⁴, 5×10¹⁴, 1×10¹⁵, or 5×10¹⁵ wells.

In some embodiments, the plurality of beads comprises at least the first bead comprising the first plurality of oligonucleotides. In some embodiments, the plurality of beads comprises at least the first bead comprising the plurality of first oligonucleotides. In some embodiments, the plurality of beads comprises at least the third bead comprising the plurality of third oligonucleotides. In some embodiments, the plurality of beads comprises at least the first bead comprising the plurality of first oligonucleotides, the second bead comprising the plurality of second oligonucleotides, and the third bead comprising the plurality of third oligonucleotides. In some embodiments, wherein the first and second wells are comprised within a plurality of N wells, wherein N is an integer, a corresponding plurality of beads comprising up to Nth beads (e.g. first, second, third . . . up to Nth) comprises at least the corresponding N pluralities of oligonucleotides (e.g., a plurality of first oligonucleotides, a plurality of second oligonucleotides, . . . , up to a plurality of Nth oligonucleotides).

In some embodiments, the oligonucleotides within the plurality of first oligonucleotides each has different regimented lengths. In some embodiments, the oligonucleotides within the plurality of second oligonucleotides each has different regimented lengths. In some embodiments, the oligonucleotides within the plurality of third oligonucleotides each has different regimented lengths. In some embodiments, the oligonucleotides within the plurality of first oligonucleotides and/or the oligonucleotides within the plurality of second oligonucleotides each has different regimented lengths. In some embodiments, the oligonucleotides within the plurality of first oligonucleotides, the oligonucleotides within the plurality of second oligonucleotides, and/or the oligonucleotides within the plurality of third oligonucleotides each has different regimented lengths. In some embodiments, the oligonucleotides in each plurality range in length from any one of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the oligonucleotides in each plurality range in length from 10 to 106 nucleotides. In some embodiments, the oligonucleotides in the plurality have regimented lengths of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the oligonucleotides in the plurality have regimented lengths of between 1 and 100 nucleotides, 100 and 1000 nucleotides or 1000 and 10,000 nucleotides. In some embodiments, the length between a shortest and a longest oligonucleotide in a plurality is about 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000 or 50,000 nucleotides. In some embodiments, the length between a shortest and a longest oligonucleotide in a plurality is any one of about 10 to 100, 10 to 500, 10 to 1000, 10 to 10000, 10 to 50000, 100 to 500, 100 to 1000, 100 to 10000, 100 to 50000, 1000 to 10000, 1000 to 50000, or 10000 to 50000 nucleotides. In some embodiments, the length between a shortest and a longest oligonucleotide in a plurality is about 100 nucleotides.

As used herein, “regimented” refers to differences in length of oligonucleotides within a population (or plurality) of oligonucleotides. A population (or plurality) of oligonucleotides having regimented lengths means that oligonucleotides in the population (or plurality) differ in length by multiples of a specified number of nucleotides. For example, a population (or plurality) of five oligonucleotides having lengths of 100, 105, 110, 115 and 120 nucleotides could be said to have a regimented length of 5 nucleotides.

In some embodiments according to any of the 3D arrays described herein, wherein the porous medium is a three-dimensional medium, the process of generating the 3D array further comprises migrating the plurality of first oligonucleotides through the three-dimensional medium in a first direction to separate individual oligonucleotides in the first plurality by size and immobilizing the separated oligonucleotides of the first plurality in the medium. In some embodiments, the process of generating the 3D array further comprises migrating the plurality of second oligonucleotides through the three-dimensional medium in a second direction to separate individual oligonucleotides in the second plurality by size and immobilizing the separated oligonucleotides of the second plurality in the medium. In some embodiments, the process of generating the 3D array further comprises migrating the plurality of third oligonucleotides through the three-dimensional medium in a third direction to separate individual oligonucleotides in the first plurality by size and immobilizing the separated oligonucleotides of the third plurality in the medium. In some embodiments according to any of the 3D arrays described herein, wherein the porous medium is a three-dimensional medium, the process of generating the 3D array further comprises: (a) migrating the plurality of first oligonucleotides through the three-dimensional medium in a first direction to separate individual oligonucleotides in the first plurality by size and immobilizing the separated oligonucleotides of the first plurality in the medium; and/or (b) migrating the plurality of second oligonucleotides through the three-dimensional medium in a second direction to separate individual oligonucleotides in the second plurality by size and immobilizing the separated oligonucleotides of the second plurality in the medium; and/or (c) migrating the plurality of third oligonucleotides through the three-dimensional medium in a third direction to separate individual oligonucleotides in the first plurality by size and immobilizing the separated oligonucleotides of the third plurality in the medium.

In some embodiments, wherein the porous material is a three-dimensional medium, the porous material is a polymer matrix. In some embodiments, the porous material is a silica matrix. In some embodiments, the porous material is a gel. In some embodiments, the porous material is a hydrogel. In some embodiments, the porous material is a polymer matrix comprising collagen, laminin and/or fibronectin. In some embodiments, the porous material is a polymer matrix comprising hydrogel. In some embodiments, the porous material is a polymer matrix comprising polyacrylamide.

In some embodiments, the migration comprises migration along a field, such as but not limited to migration along an electromagnetic field. In some embodiments, an electromagnetic field is applied across the medium to migrate the oligonucleotides through the medium. In some embodiments, the migration comprises electrophoresis.

In some embodiments, wherein prior to the migrating and the immobilizing of the pluralities of oligonucleotides, the process of generating the 3D array comprises embedding a cell sample and/or a tissue sample in the three-dimensional medium (such as a polymer matrix). In some embodiments, wherein prior to the migrating and the immobilizing of the pluralities of oligonucleotides, the process of generating the 3D array comprises embedding a cell sample and/or a tissue sample in the three-dimensional medium (such as a polymer matrix). In some embodiments, the medium contains cellular RNAs. In some embodiments, wherein prior to the migrating and the immobilizing of the pluralities of oligonucleotides, the process of generating the 3D array comprises capturing cellular RNAs in the medium using RNA capturing probes and/or production of RNA-derived amplification products.

In some embodiments, the oligonucleotides of the first, second and third pluralities comprise a hybridizing nucleotide sequence capable of identifying the same cellular RNA. In some embodiments, the oligonucleotides of all pluralities comprise a hybridizing nucleotide sequence capable of identifying the same cellular RNA.

In some embodiments, the oligonucleotides of the first, second and third pluralities comprise hybridizing nucleotide sequences capable of identifying different cellular RNAs. In some embodiments, the oligonucleotides of all pluralities comprise hybridizing nucleotide sequences capable of identifying different cellular RNAs.

In some embodiments according to any one of the 3D arrays described above, the process of generating the 3D array further comprises subjecting the medium containing the separated oligonucleotides to conditions where the hybridizing nucleotide sequence of the separated oligonucleotides hybridizes to the captured cellular RNAs, RNA capturing probes and/or RNA-derived amplification products present in the medium that have a nucleotide sequence complementary to the hybridizing nucleotide sequence of the oligonucleotides.

In some embodiments, provided is a 3D array and the use thereof, wherein the 3D array is generated in steps comprising: (a) capturing cellular RNAs in a three-dimensional tissue sample using RNA capturing probes and/or sequence-specific amplification of the cellular RNAs; (b) embedding the tissue sample in a conductive polymer; (c) partitioning a plurality of beads into wells on a substrate, wherein a first bead in a first well comprises a plurality of first oligonucleotides of varying lengths which comprise the same first barcode sequence, a second bead in a second well comprises a plurality of second oligonucleotides of varying lengths which comprise the same second barcode sequence and a third bead in a third well comprises a plurality of third oligonucleotides of varying lengths which comprise the same third barcode sequence; further wherein the first, second and third pluralities of oligonucleotides encode i) hybridizing nucleotide sequences capable of identifying different RNAs, or ii) nucleotide sequences capable of hybridizing to RNA, RNA capturing probes, or the RNA-derived amplification products that are different; (d) placing the conductive polymer abutting the wells on the substrate; (e) disrupting the first, second and third beads to release the oligonucleotides in the corresponding wells, wherein the released oligonucleotides are allowed to migrate into the conductive polymer abutting the wells, wherein a position of a particular released oligonucleotide in the direction correlates with the length of the particular released oligonucleotide; (f) electrophoresing the first plurality of oligonucleotides through a first dimension of the conductive polymer to separate individual oligonucleotides of the first composition by size, and immobilizing the separated oligonucleotides of the first plurality in the polymer; electrophoresing the second plurality of oligonucleotides through a second dimension of the conductive polymer to separate individual oligonucleotides of the second composition by size, and immobilizing the separated oligonucleotides of the second plurality in the polymer; electrophoresing the second plurality of oligonucleotides through a third dimension of the conductive polymer to separate individual oligonucleotides of the third composition by size, and immobilizing the separated oligonucleotides of the third pluralities in the polymer; (g) subjecting the conductive polymer to conditions providing for hybridization of oligonucleotides in the polymer to the captured RNAs, RNA capturing probes and/or RNA-derived amplification products; and wherein the use of the 3D-array comprises (h) performing nucleotide sequencing of the hybridized oligonucleotides and captured RNAs, RNA capturing probes and/or RNA-derived amplification products; and (i) integrating sizes of the sequenced oligonucleotides with identity of the captured cellular RNAs to determine relative location of the cellular RNAs in the three-dimensions of the polymer.

In some embodiments, the oligonucleotides are gradient-tagging, wherein migration of the oligonucleotides creates a concentration gradient along the migration path. In instances where the barcoded probes on an array are generated through ligation of two or more oligonucleotides, a concentration gradient of the oligonucleotides can be applied to a substrate such that different combinations of the oligonucleotides are incorporated into a barcoded probe depending on its location on the substrate.

In some embodiments, the directions or dimensions of migration for any of two pluralities of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the directions or dimensions of migration for any of two pluralities of oligonucleotides intersect orthogonally or perpendicularly at an angle of 90 degrees, or about orthogonally or perpendicularly at an angle of about 90 degrees.

In some embodiments, the direction or dimension of migration for the first plurality of oligonucleotides and the direction or dimension of migration for the second plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the first plurality of oligonucleotides and the direction or dimension of migration for the second plurality of oligonucleotides intersect orthogonally or perpendicularly at an angle of 90 degrees, or about orthogonally or perpendicularly at an angle of about 90 degrees.

In some embodiments, the direction or dimension of migration for the first plurality of oligonucleotides and the direction or dimension of migration for the third plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the first plurality of oligonucleotides and the direction or dimension of migration for the third plurality of oligonucleotides intersect orthogonally or perpendicularly at an angle of 90 degrees, or about orthogonally or perpendicularly at an angle of about 90 degrees.

In some embodiments, the direction or dimension of migration for the second plurality of oligonucleotides and the direction or dimension of migration for the third plurality of oligonucleotides intersect at an angle of about 85, about 80, about 75, about 70, about 65, about 60, about 55, about 50, about 45, about 40, about 35, about 30, about 25, about 20, about 15, about 10, or about 5 degrees. In some embodiments, the direction or dimension of migration for the second plurality of oligonucleotides and the direction or dimension of migration for the third plurality of oligonucleotides intersect orthogonally or perpendicularly at an angle of 90 degrees, or about orthogonally or perpendicularly at an angle of about 90 degrees.

In some embodiments, the directions or dimensions of migration for the first, second and third pluralities of oligonucleotides intersect one another at an angle of about 85, about 80, about 75, about 70, about 65, about 60, about 55, about 50, about 45, about 40, about 35, about 30, about 25, about 20, about 15, about 10, or about 5 degrees. In some embodiments, the directions or dimensions of migration for the first, second and third pluralities of oligonucleotides intersect one another at an angle of 90 degrees, or about orthogonally or perpendicularly at an angle of about 90 degrees.

D. Spatial Analysis

In particular embodiments, provided herein are kits and compositions for spatial array-based analysis of biological samples. Array-based spatial analysis methods involve the transfer of one or more analytes or proxies thereof from a biological sample to an array of features on a substrate, where each feature is associated with a spatial location on the array. Subsequent analysis of the transferred analytes or proxies thereof includes determining the identity of the analytes and the spatial location of each analyte within the biological sample. The spatial location of each analyte within the biological sample is determined based on the feature to which each analyte is bound on the array, and the feature's relative spatial location within the array. In some embodiments, the array of features on a substrate comprise a spatial barcode that corresponds to the feature's relative spatial location within the array. Each spatial barcode of a feature may further comprise a fluorophore, to create a fluorescent hybridization array. A feature may comprise UMIs that are generally unique per nucleic acid molecule in the feature—this is so the number of unique molecules can be estimated, as opposed to an artifact in experiments or PCR amplification bias that drives amplification of smaller, specific nucleic acid sequences.

In some embodiments, an oligonucleotide probe can directly capture an analyte, such as mRNAs based on a poly(dT) capture domain on the oligonucleotide probe immobilized on an array. In some embodiments, the oligonucleotide probe is used for indirect analyte capture. For example, in fixed samples, such as FFPE, a probe pair can be used, and probes pairs can be target specific for each gene of the transcriptome. The probe pairs are delivered to a tissue section (which is itself on a spatial array) with a decrosslinking agent and a ligase, and the probe pairs are left to hybridize and ligate, thereby forming ligation products. The ligation products contain sequences in one or more overhangs of the probes, and the overhangs are not target specific and are complementary to capture domains on oligonucleotides immobilized on a spatial array, thus allowing the ligation product (which is a proxy for the analyte) to be captured on the array, processed, and subsequently analyzed (e.g., using a sequencing method).

In particular embodiments, the kits and compositions for spatial array-based analysis provide for the detection of differences in an analyte level (e.g., gene and/or protein expression) within different cells in a tissue of a mammal or within a single cell from a mammal. For example, the kits and compositions can be used to detect the differences in analyte levels (e.g., gene and/or protein expression) within different cells in histological slide samples (e.g., intact tissue section), the data from which can be reassembled to generate a three-dimensional map of analyte levels (e.g., gene and/or protein expression) of a tissue sample obtained from a mammal, e.g., with a degree of spatial resolution (e.g., single-cell scale resolution).

In some embodiments, an array generated using a method disclosed herein can be used in array-based spatial analysis methods which involve the transfer of one or more analytes from a biological sample to an array of features on a substrate, each of which is associated with a spatial location on the array. Subsequent analysis of the transferred analytes includes determining the identity of the analytes and the spatial location of each analyte within the sample. The spatial location of each analyte within the sample is determined based on the feature to which each analyte is bound in the array, and the feature's relative spatial location within the array.

There are at least two general methods to associate a spatial barcode with one or more neighboring cells, such that the spatial barcode identifies the one or more cells, and/or contents of the one or more cells, as associated with a particular spatial location. One general method is to drive target analytes out of a cell and towards the spatially-barcoded array. In some embodiments, the spatially-barcoded array populated with capture probes is contacted with a sample, and sample is permeabilized, allowing the target analyte or proxies thereof to migrate away from the sample and toward the array. The target analytes or proxies thereof interact with a capture probe on the spatially-barcoded array. Once the target analyte or proxy hybridizes/is bound to the capture probe, the sample is optionally removed from the array and the capture probes are analyzed in order to obtain spatially-resolved analyte information. Methods for performing such spatial analysis of tissue sections are known in the art and include but are not limited to those methods disclosed in U.S. Pat. Nos. 10,030,261, 11,332,790 and US Patent Pub No. 20220127672 and US Patent Pub No. 20220106632, the contents of which are herein incorporated by reference in their entireties.

Another general method is to cleave the spatially-barcoded capture probes from an array, and drive the spatially-barcoded capture probes towards and/or into or onto the sample. In some embodiments, the spatially-barcoded array populated with capture probes is contacted with a sample. The spatially-barcoded capture probes are cleaved and then interact with cells within the provided sample (See, for example, U.S. Pat. No. 11,352,659 the contents of which are herein incorporate by reference in its entirety). The interaction can be a covalent or non-covalent cell-surface interaction. The interaction can be an intracellular interaction facilitated by a delivery system or a cell penetration peptide. Once the spatially-barcoded capture probe is associated with a particular cell, the sample can be optionally removed for analysis. The sample can be optionally dissociated before analysis. Once the tagged cell is associated with the spatially-barcoded capture probe, the capture probes can be analyzed (e.g., by sequencing) to obtain spatially-resolved information about the tagged cell.

Sample preparation may include placing the sample on a slide, fixing the sample, and/or staining the sample for imaging. The stained sample may be imaged on the array using both brightfield (to image the sample hematoxylin and eosin stain) and/or fluorescence (to image features) modalities. In some embodiments, target analytes are then released from the sample and capture probes forming the spatially-barcoded array hybridize or bind the released target analytes. The sample is then removed from the array and the capture probes cleaved from the array. The sample and array are then optionally imaged a second time in one or both modalities (brightfield and fluorescence) while the analytes are reverse transcribed into cDNA, and an amplicon library is prepared and sequenced. The two sets of images can then be spatially-overlaid in order to correlate spatially-identified sample information. When the sample and array are not imaged a second time, a spot coordinate file may be supplied. The spot coordinate file can replace the second imaging step. Further, amplicon library preparation can be performed with a unique PCR adapter and sequenced.

In some embodiments, a spatially-labelled array on a substrate is used, where capture probes labelled with spatial barcodes are clustered at areas called features. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatially-barcoded array is contacted with a sample, and the sample is permeabilized through application of permeabilization reagents. Permeabilization reagents may be administered by placing the array/sample assembly within a bulk solution. Alternatively, permeabilization reagents may be administered to the sample via a diffusion-resistant medium and/or a physical barrier such as a lid, wherein the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration. As another example, analyte migration can be active migration, using an electrophoretic transfer system, for example. Once the analytes are in close proximity to the spatially-barcoded capture probes, the capture probes can hybridize or otherwise bind a target analyte. The sample can be optionally removed from the array.

Adapters and assay primers can be used to allow the capture probe or the analyte capture agent to be attached to any suitable assay primers and used in any suitable assays. A capture probe that includes a spatial barcode can be attached to a bead that includes a poly(dT) sequence. A capture probe including a spatial barcode and a poly(T) sequence can be used to assay multiple biological analytes as generally described herein (e.g., the biological analyte includes a poly(A) sequence or is coupled to or otherwise is associated with an analyte capture agent comprising a poly(A) sequence as the analyte capture sequence).

The capture probes can be optionally cleaved from the array, and the captured analytes can be spatially-tagged by performing a reverse transcriptase first strand cDNA reaction. A first strand cDNA reaction can be optionally performed using template switching oligonucleotides. For example, a template switching oligonucleotide can hybridize to a poly(C) tail added to a 3′end of the cDNA by a reverse transcriptase enzyme. The original mRNA template and template switching oligonucleotide can then be denatured from the cDNA and the barcoded capture probe can then hybridize with the cDNA and a complement of the cDNA can be generated. The first strand cDNA can then be purified and collected for downstream amplification steps. The first strand cDNA can be amplified using PCR, wherein the forward and reverse primers flank the spatial barcode and target analyte regions of interest, generating a library associated with a particular spatial barcode. In some embodiments, the cDNA comprises a sequencing by synthesis (SBS) primer sequence. The library amplicons are sequenced and analyzed to decode spatial information.

In some embodiments, the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. Once the capture probes capture the target analyte(s), first strand cDNA created by template switching and reverse transcriptase is then denatured and the second strand is then extended. The second strand cDNA is then denatured from the first strand cDNA, neutralized, and transferred to a tube. cDNA quantification and amplification can be performed using standard techniques discussed herein. The cDNA can then be subjected to library preparation and indexing, including fragmentation, end-repair, and a-tailing, and indexing PCR steps, and then sequenced.

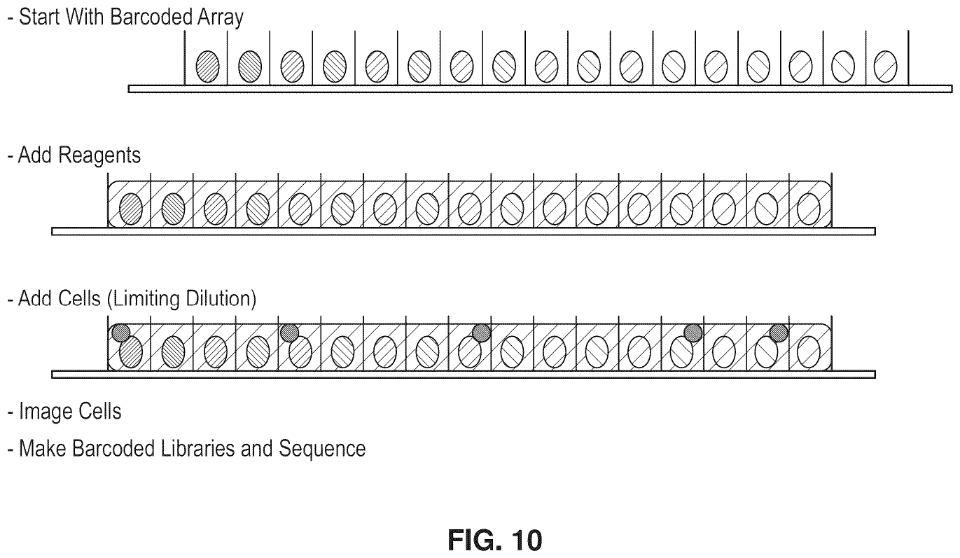

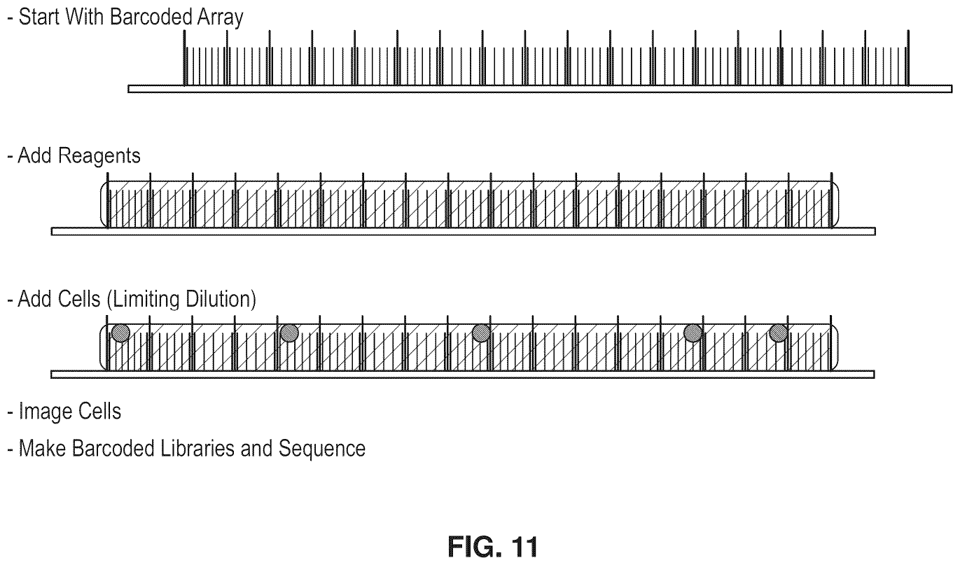

Microwell based single cell approaches frequently rely on trapping a single cell and a single barcoded bead into a subset of the wells to produce barcoded single cell libraries. In such an approach, the barcodes are randomly distributed into the wells because the beads are randomly deposited into wells. Provided herein is a method in which there are one or more known barcodes in each well, and the method comprises associating (e.g., mapping) optical measurements of cells taken on a microwell array to the corresponding barcode(s). The method can be used to connect single cell phenotyping information gathered via microscopy approaches with single cell sequencing data. In some embodiments, a microwell array containing barcoded beads is generated, to provide a microwell array with position-fixed beads and known barcode locations. The locations of these beads can be decoded (e.g., during manufacturing of the array), and cells can be loaded onto the bead array (e.g., by a user), e.g., as illustrated in FIG. 10. In some embodiments, an array containing patterned barcodes in a known spatial pattern on a surface is generated (e.g., through a method disclosed herein, such as those in Section II). For instance, oligonucleotides can be attached to molecules on the substrate to generate barcodes of known patterns on the array, and a microwell can be aligned on top of the array, and cells can be loaded onto the microwells (e.g., by a user), e.g., as illustrated in FIG. 11.

### V. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

As used in this specification and the appended claims, the singular forms “a,” “an” and “the” include plural referents unless the context clearly dictates otherwise. Thus, for example, reference to “a molecule” includes a plurality of such molecules, and the like.

The term “barcode,” comprises a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be added to, for example, a fragment of a deoxyribonucleic acid (DNA) or ribonucleic acid (RNA) sample before or during sequencing of the sample. Barcodes can allow for identification and/or quantification of individual sequencing-reads (e.g., a barcode can be or can include a unique molecular identifier or “UMI”).

Barcodes can spatially-resolve molecular components found in biological samples, for example, at single-cell scale resolution (e.g., a barcode can be or can include a “spatial barcode”). In some embodiments, a barcode includes both a UMI and a spatial barcode. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

As used herein, the term “substrate” generally refers to a substance, structure, surface, material, means, or composition, which comprises a nonbiological, synthetic, nonliving, planar, spherical or flat surface. The substrate may include, for example and without limitation, semiconductors, synthetic metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics; nanostructures and microstructures. The substrate may comprises an immobilization matrix such as but not limited to, insolubilized substance, solid phase, surface, layer, coating, woven or nonwoven fiber, matrix, crystal, membrane, insoluble polymer, plastic, glass, biological or biocompatible or bioerodible or biodegradable polymer or matrix, microparticle or nanoparticle. Other example may include, for example and without limitation, monolayers, bilayers, commercial membranes, resins, matrices, fibers, separation media, chromatography supports, polymers, plastics, glass, mica, gold, beads, microspheres, nanospheres, silicon, gallium arsenide, organic and inorganic metals, semiconductors, insulators, microstructures and nanostructures. Microstructures and nanostructures may include, without limitation, microminiaturized, nanometer-scale and supramolecular probes, tips, bars, pegs, plugs, rods, sleeves, wires, filaments, and tubes.

As used herein, the term “nucleic acid” generally refers to a polymer comprising one or more nucleic acid subunits or nucleotides. A nucleic acid may include one or more subunits selected from adenosine (A), cytosine (C), guanine (G), thymine (T) and uracil (U), or variants thereof. A nucleotide can include A, C, G, T or U, or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (e.g., A or G, or variant thereof) or a pyrimidine (e.g., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double-stranded.

The term “nucleic acid sequence” or “nucleotide sequence” as used herein generally refers to nucleic acid molecules with a given sequence of nucleotides, of which it may be desired to know the presence or amount. The nucleotide sequence can comprise ribonucleic acid (RNA) or DNA, or a sequence derived from RNA or DNA. Examples of nucleotide sequences are sequences corresponding to natural or synthetic RNA or DNA including genomic DNA and messenger RNA. The length of the sequence can be any length that can be amplified into nucleic acid amplification products, or amplicons, for example, up to about 20, 50, 100, 200, 300, 400, 500, 600, 700, 800, 1000, 1200, 1500, 2000, 5000, 10000 or more than 10000 nucleotides in length, or at least about 20, 50, 100, 200, 300, 400, 500, 600, 700, 800, 1000, 1200, 1500, 2000, 5000, 10000 nucleotides in length.

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

As used herein, the term “adjacent” or “adjacent to,” includes “next to,” “adjoining,” and “abutting.” In one example, a first location is adjacent to a second location when the first location is in direct contact and shares a common border with the second location and there is no space between the two locations. In some cases, the adjacent is not diagonally adjacent.

An “adaptor,” an “adapter,” and a “tag” are terms that are used interchangeably in this disclosure, and refer to species that can be coupled to a polynucleotide sequence (in a process referred to as “tagging”) using any one of many different techniques including (but not limited to) ligation, hybridization, and tagmentation. Adaptors can also be nucleic acid sequences that add a function, e.g., spacer sequences, primer sequences/sites, barcode sequences, unique molecular identifier sequences.

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

A “proximity ligation” is a method of ligating two (or more) nucleic acid sequences that are in proximity with each other through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference).

A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

As used herein, the term “splint” is an oligonucleotide that, when hybridized to other polynucleotides, acts as a “splint” to position the polynucleotides next to one another so that they can be ligated together. In some embodiments, the splint is DNA or RNA. The splint can include a nucleotide sequence that is partially complementary to nucleotide sequences from two or more different oligonucleotides. In some embodiments, the splint assists in ligating a “donor” oligonucleotide and an “acceptor” oligonucleotide. In general, an RNA ligase, a DNA ligase, or another other variety of ligase is used to ligate two nucleotide sequences together.

In some embodiments, the splint is between 10 and 50 oligonucleotides in length, e.g., between 10 and 45, 10 and 40, 10 and 35, 10 and 30, 10 and 25, or 10 and 20 oligonucleotides in length. In some embodiments, the splint is between 15 and 50, 15 and 45, 15 and 40, 15 and 35, 15 and 30, 15 and 30, or 15 and 25 nucleotides in length.

A “feature” is an entity that acts as a support or repository for various molecular entities used in sample analysis. In some embodiments, some or all of the features in an array are functionalized for analyte capture. In some embodiments, functionalized features include one or more capture probe(s). Examples of features include, but are not limited to, a bead, a spot of any two- or three-dimensional geometry (e.g., an ink jet spot, a masked spot, a square on a grid), a well, and a hydrogel pad. In some embodiments, features are directly or indirectly attached or fixed to a substrate. In some embodiments, the features are not directly or indirectly attached or fixed to a substrate, but instead, for example, are disposed within an enclosed or partially enclosed three dimensional space (e.g., wells or divots).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “template” as used herein generally refers to individual polynucleotide molecules from which another nucleic acid, including a complementary nucleic acid strand, can be synthesized by a nucleic acid polymerase. In addition, the template can be one or both strands of the polynucleotides that are capable of acting as templates for template-dependent nucleic acid polymerization catalyzed by the nucleic acid polymerase. Use of this term should not be taken as limiting the scope of the present disclosure to polynucleotides which are actually used as templates in a subsequent enzyme-catalyzed polymerization reaction. The template can be an RNA or DNA. The template can be cDNA corresponding to an RNA sequence. The template can be DNA.

As used herein, “amplification” of a template nucleic acid generally refers to a process of creating (e.g., in vitro) nucleic acid strands that are identical or complementary to at least a portion of a template nucleic acid sequence, or a universal or tag sequence that serves as a surrogate for the template nucleic acid sequence, all of which are only made if the template nucleic acid is present in a sample. Typically, nucleic acid amplification uses one or more nucleic acid polymerase and/or transcriptase enzymes to produce multiple copies of a template nucleic acid or fragments thereof, or of a sequence complementary to the template nucleic acid or fragments thereof. In vitro nucleic acid amplification techniques are may include transcription-associated amplification methods, such as Transcription-Mediated Amplification (TMA) or Nucleic Acid Sequence-Based Amplification (NASBA), and other methods such as Polymerase Chain Reaction (PCR), Reverse Transcriptase-PCR (RT-PCR), Replicase Mediated Amplification, and Ligase Chain Reaction (LCR).

In addition to those above, a wide variety of other features can be used to form the arrays described herein. For example, in some embodiments, features that are formed from polymers and/or biopolymers that are jet printed, screen printed, or electrostatically deposited on a substrate can be used to form arrays.

### EXAMPLES

The following examples are included for illustrative purposes only and are not intended to limit the scope of the disclosure.

### Example 1: Generation of Spatial Array by Using Etching to Generate Micro-Partitions

This example provides an exemplary method for barcode assembly following well etching and one round of gel bead partitioning and disruption, on immobilized oligonucleotides (e.g., capture sequence) partitioned in the wells. In particular, this example describes the generation of a barcoded oligonucleotide array using micro-partition and gel bead delivery.

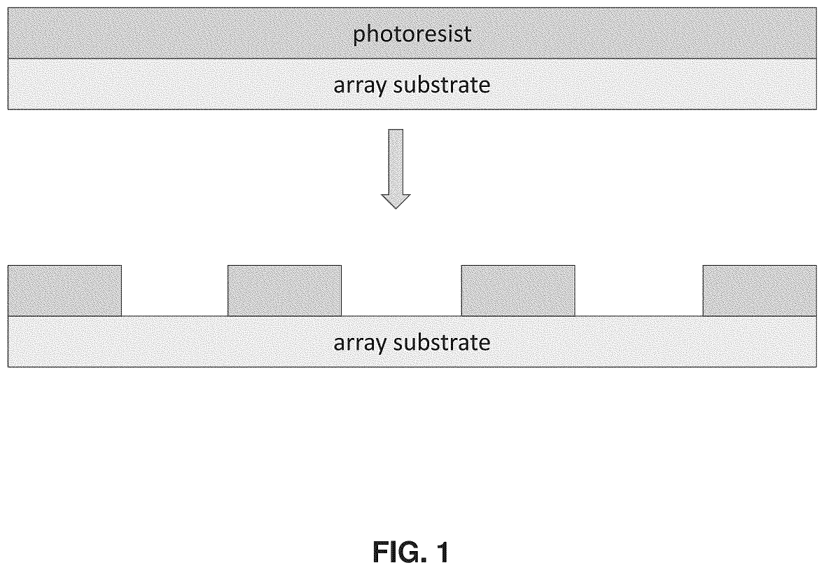

FIG. 1 shows a method of generating micro-partitions. An array substrate (such as a glass wafer) is first coated with a photoresist (such as a positive photoresist), such as by spin-coating (upper figure). By way of etching, regions of photoresist are removed thereby generating etched wells on top of the array substrate.

As shown in FIG. 2A, Round 1 gel beads comprising Round 1 oligonucleotides comprising distinct barcodes (BC1, BC2, BC3) are introduced into the etched wells. Utilizing the pattern of well etching, the barcoded gel beads are thus micro-partitioned into the etched wells.

Subsequent to the introduction of gel beads into the etched wells, the gel beads are disrupted, thereby releasing the Round 1 oligonucleotides into the etched wells (FIG. 2B).

In one embodiment, the wells could contain a capture sequence arrayed before photoresist etching (not shown), or arrayed subsequent to etching of the wells. Upon the introduction of Round 1 gel beads and disruption of the gel beads, the Round 1 oligonucleotides (BC1) are released into the etched wells, wherein the Round 1 oligonucleotides (BC1) are hybridized and/or ligated to the capture sequence inside the etched wells, to form extended nucleic acids (FIG. 3).

In another embodiment, the wells contain nucleic acid molecules comprising a capture sequence (e.g., partial R1) and the nucleic acid molecules comprise sequences hybridized to Round 1 splints, which in turn hybridized to the Round 1 oligonucleotides containing barcodes (e.g., barcode 1), to form extended nucleic acids (FIG. 4).

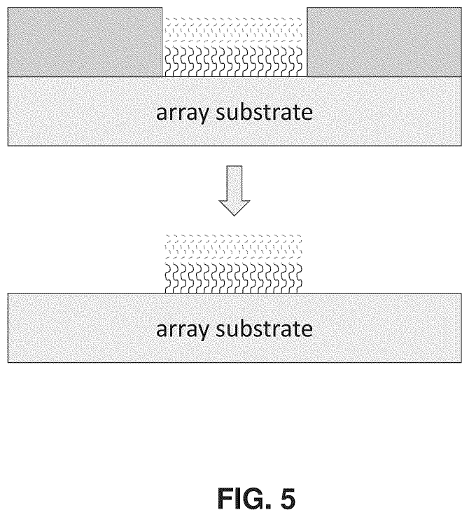

Subsequent to the generation of extended nucleic acids, the photoresist can be removed, e.g., degraded with irradiation, while the extended nucleic acids remained associated with the region corresponding to the well (FIG. 5).

### Example 2: Generation of an Array

This example demonstrates a successful barcode assembly following well etching and two rounds of gel bead partitioning and disruption, on immobilized oligonucleotides (e.g., capture sequence) partitioned in the wells. In particular, this example describes the generation of a combinatorial oligonucleotide array using microscale partitions and gel bead delivery.

Microscale partitions are generated as described in Example 1 and FIG. 1. By way of etching, regions of photoresist are removed thereby generating etched wells on top of the array substrate.

As shown in FIGS. 6A-6B, Round 1 gel beads comprising Round 1 oligonucleotides comprising distinct barcodes (BC1 part A, BC2 part A, BC3 part A) are introduced into the etched wells. Utilizing the pattern of well etching, the barcoded gel beads are thus micro-partitioned into the etched wells.

Subsequent to the introduction of Round 1 gel beads into the etched wells, the gel beads are disrupted, thereby releasing the Round 1 oligonucleotides into the etched wells (part A nucleotides). Similar to Example 1, Round 1 oligonucleotides (part A) are hybridized and/or ligated to the capture sequence inside the etched wells, to form extended nucleic acids.

Subsequently, Round 2 gel beads comprising Round 2 oligonucleotides comprising distinct barcodes (BC1 part B, BC2 part B, BC3 part B) are introduced into the corresponding etched wells. Similarly, the barcoded gel beads are thus micro-partitioned into the etched wells. The Round 2 gel beads are disrupted, thereby releasing the Round 1 oligonucleotides into the etched wells (part B nucleotides). The Round 2 oligonucleotides are then hybridized and/or ligated to the extended nucleic acids (capture sequence x BC part A) inside the etched wells, to form further extended nucleic acids (capture sequence x BC part A x BC part B).

There is a low probability rate of “cross” product between adjacent wells due to diffusion of oligonucleotides to neighboring wells, such as (BC1-part A x BC2-part B and BC2-part A x BC1 part B). These occurrences can further provide proximity information of where the dominant (high #UMI) barcodes are clustered. Using this information, the full position map of the dominant barcodes can be reconstructed.

### Example 3: Generation of Master Arrays and Copy Arrays

This example describes a barcode assembly (master) and generation of copy arrays, following well etching and one round of gel bead partitioning and disruption, on immobilized oligonucleotides (e.g., capture sequence) partitioned in the wells. In particular, this example describes the generation of a master-copy oligonucleotide arrays using micro-partition and gel bead delivery.

Micro-partitions are generated as described in Example 1 and FIG. 1. By way of etching, regions of photoresist are removed thereby generating etched wells on top of the array substrate.

Similar to Example 1 and FIGS. 2A-2B, Round 1 gel beads comprising Round 1 oligonucleotides comprising distinct barcodes (BC1, BC2, BC3) are introduced into the etched wells. Utilizing the pattern of well etching, the barcoded gel beads are thus micro-partitioned into the etched wells, thus generating the master array.

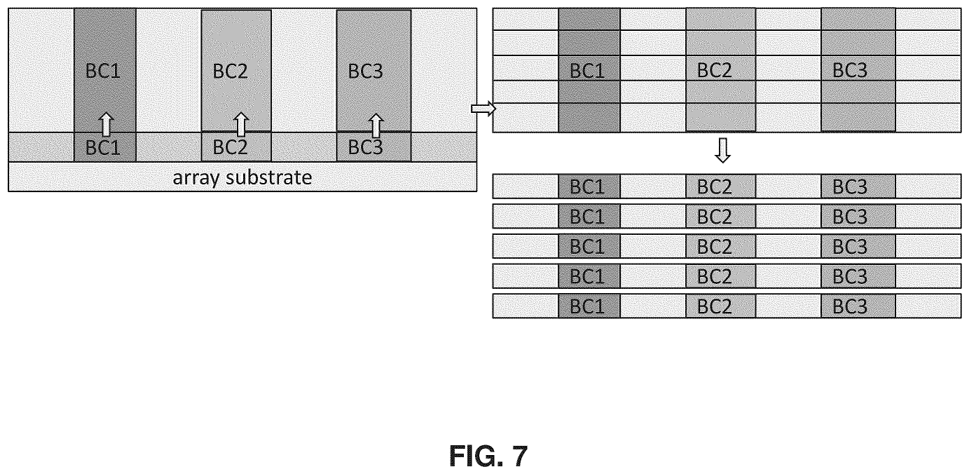

The nucleic acids in the wells are cleaved from the substrate or are subjected to amplification (e.g., via PCR). The released nucleic acids and/or the amplification products are migrated into a porous material (such as a hydrogel) abutting the wells, with use of electrophoresis (FIG. 7, left panel).

As shown in FIG. 7 upper right panel, the porous materials (such as a hydrogel) are then divided into subparts (e.g. cut into gel slabs), along one or more planes that intersect the migration paths of the nucleic acids.

As shown in FIG. 7 lower right panel, the subdivisions of gel slabs are separated, with each gel slab constituting a copy of the master array.

### Example 4: Generation of 3-Dimensional Spatial Oligonucleotide Array

This example describes a barcode assembly and generation of a 3-dimensional spatial array, following well etching and one round of gel bead partitioning and disruption, on immobilized oligonucleotides (e.g., with a capture sequence) partitioned in the wells. In particular, this example describes the micro-partitioning and subsequent 3-dimensional migration of barcoded-oligonucleotides with variable length to generate a 3-dimensional spatial oligonucleotide array.

Micro-partitions are generated as described in Example 1 and FIG. 1. By way of etching, regions of photoresist are removed thereby generating etched wells on top of the array substrate.

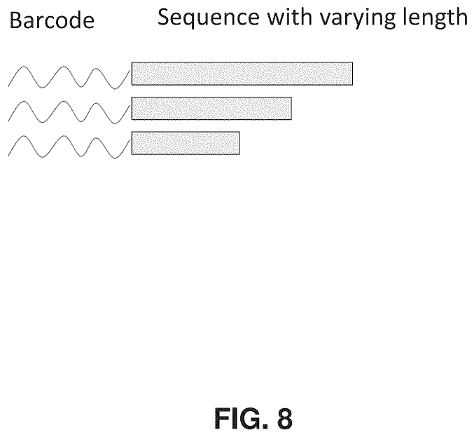

Similar to Example 1 and FIGS. 2A-2B, Round 1 gel beads comprising Round 1 oligonucleotides comprising distinct barcodes (BC1, BC2, BC3) are introduced into the etched wells. Utilizing the pattern of well etching, the barcoded gel beads are thus micro-partitioned into the etched wells, thus generating the master array. Notably, each plurality of oligonucleotides comprising the same barcode (e.g. BC1) contained a population of oligonucleotides comprising a distribution of variable lengths (FIG. 8).

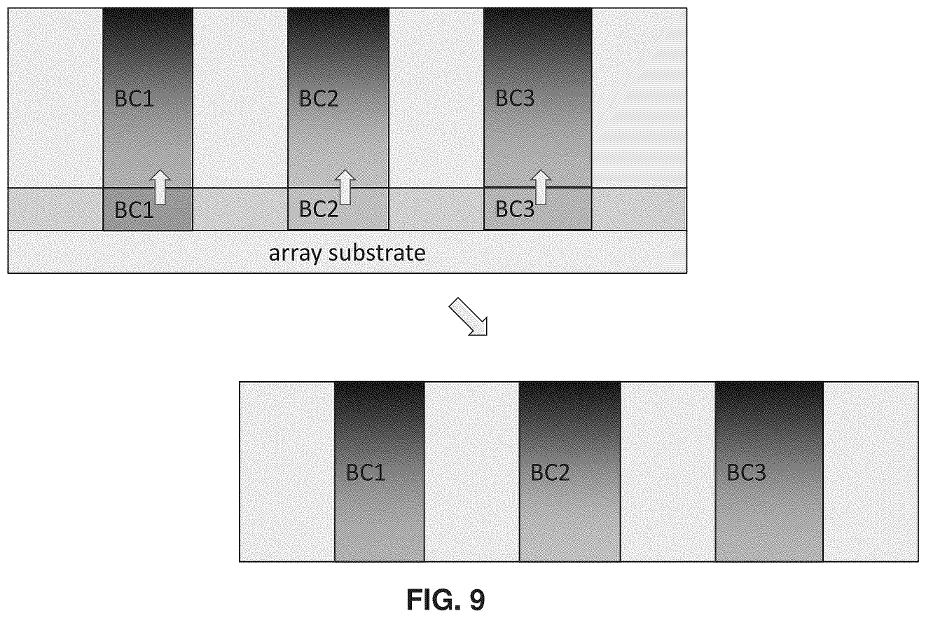

The nucleic acids in the wells are then cleaved from the substrate or could be subjected to amplification (e.g., via PCR). The released nucleic acids and/or the amplification products are migrated into a porous material (such as a hydrogel) abutting the wells, with use of electrophoresis (FIG. 9, upper panel). The oligonucleotides of longer length traveled further along the path of migration, thereby encoding the z-dimensional information for the barcoded oligonucleotide populations in the porous hydrogel. In an optional example (not shown), electrophoresis could be further refined to direct migration of different oligonucleotide populations along different desired axes within the hydrogel (such as X-axis, Y-axis and Z-axis) thereby further expanding the z-dimensional information available for the barcoded oligonucleotide populations.

As shown in FIG. 9 lower panel, the hydrogel is then removed, thereby generating a 3-dimensional spatial oligonucleotide array.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1-69. (canceled)

70. A method for providing an array, comprising:
(a) partitioning a plurality of Round 1 beads into wells on a substrate, wherein the Round 1 bead in a first well and the Round 1 bead in a second well each comprises a different Round 1 oligonucleotide;
(b) disrupting the Round 1 beads to release the Round 1 oligonucleotides, wherein the released Round 1 oligonucleotides are attached to nucleic acid molecules in the corresponding well via ligation to generate extended nucleic acid molecules;
(c) partitioning a plurality of Round 2 beads into wells on the substrate, wherein the Round 2 bead in the first well and the Round 2 bead in the second well each comprises a different Round 2 oligonucleotide; and
(d) disrupting the Round 2 beads to release the Round 2 oligonucleotides, wherein the released Round 2 oligonucleotides are attached to the extended nucleic acid molecules in the corresponding well via ligation to generate further extended nucleic acid molecules.

71. The method of claim 70, wherein the Round 2 oligonucleotides are at least four nucleotides in length.

72. The method of claim 70, wherein the released Round 2 oligonucleotides in the first and second wells comprise a first and second Round 2 barcode sequence, respectively, wherein the first and second Round 2 barcode sequences are different from each other.

73. The method of claim 70, wherein the released Round 2 oligonucleotide comprises a sequence that hybridizes to a Round 2 splint which in turn hybridizes to the extended nucleic acid molecules in a particular well, and wherein the released Round 2 oligonucleotide is ligated to the extended nucleic acid molecules using the Round 2 splint as a template to generate the further extended nucleic acid molecules.

74. The method of claim 70, wherein the released Round 1 oligonucleotides each individually comprises a sequence that hybridizes to a Round 1 splint which in turn hybridizes to the nucleic acid molecules in a particular well, and wherein the released Round 1 oligonucleotides are ligated to the nucleic acid molecules using the Round 1 splint as a template to generate the extended nucleic acid molecules.

75. The method of claim 74 wherein the Round 1 splint is comprised in the Round 1 bead and released upon disruption of the bead.

76. The method of claim 73, wherein the Round 2 splint is common between different wells.

77. The method of claim 73, wherein the Round 2 splint is comprised in the Round 2 bead and released upon disruption of the Round 2 bead.

78. The method of claim 73, wherein the Round 2 splint is not comprised in the Round 2 bead and is separately delivered to the wells.

79. The method of claim 70, wherein the released Round 2 oligonucleotide comprises a sequence that hybridizes to a Round 3 splint which in turn hybridizes to a Round 3 oligonucleotide, and wherein the Round 3 oligonucleotide is ligated to the further extended nucleic acid molecules using the Round 3 splint as template to generate even further extended nucleic acid molecules.

80. The method of claim 70, wherein the Round 1 oligonucleotide and/or the Round 2 oligonucleotide each comprises a capture sequence.

81. The method of claim 70, wherein prior to the partitioning in step (a), the substrate is coated with a photoresist layer.

82. The method of claim 70, wherein prior to the partitioning in step (a), the substrate is coated with a photoresist layer by dipping or spin coating.

83. The method of claim 70, wherein the wells are formed by etching a layer of a photoresist on the substrate.

84. The method of claim 81, further comprising removing the photoresist, leaving the further extended nucleic acid molecules immobilized on the substrate.

85. The method of claim 80, wherein the Round 1 oligonucleotide and the Round 2 oligonucleotide each comprises a different capture sequence, wherein each capture sequence is designed to couple to one or more analytes.

86. The method of claim 70, wherein the further extended nucleic acid molecules migrate into a porous material abutting the wells.

87. The method of claim 86, wherein the porous material is a gel and the migration comprises electrophoresis.

88. The method of claim 86, wherein the migration paths in the porous material are substantially parallel to one another, and the porous material is divided into subparts along one or more planes intersecting the migration paths, thereby generating copies of the array.

89. The method of claim 86, wherein the porous material is divided into subparts along one or more planes that are substantially perpendicular to the mean migration direction.
